(19) United States
(12) Patent Application Publication
(10) Pub. No.: US 2024/0180983 A1
(43) Pub. Date: Jun. 6, 2024

(54) ADENOVIRUS ENCODING IL-15

(71) Applicant: Akamis Bio Limited, Abingdon (GB)

(72) Inventors: Brian Robert CHAMPION, Abingdon (GB); Manuela ZONCA, Abingdon (GB)

(21) Appl. No.: 18/546,357

(22) PCT Filed: Feb. 14, 2022
(86) PCT No.: PCT/EP2022/053477

§ 371 Date: Aug. 14, 2023

(30) Foreign Application Priority Data
Feb. 13, 2021 (GB) 2102049.0

(51) Int. Cl. A61K 35/761; A61K 45/06; A61P 35/00; C07K 14/52; C07K 14/54

(52) U.S. Cl. CPC A61K 35/761; A61K 45/06; A61P 35/00; C07K 14/521; C07K 14/5434; C07K 14/5443; C12N 2710/10333

(57) ABSTRACT

A group B adenovirus comprising a sequence of formula 5′ITR—B₁-B_{A}-B₂-B_{X}-B_{B}-B_{Y}-B₃-3′ITR, wherein: BY comprises a sequence -G1-G2ₙ-G3ₘ-G4ₚ-G5_{q}. G1 is a first transgene. G2 is a second transgene. G3 is a third transgene. G4 is a fourth transgene. G5 is a fifth transgene and IL-15 is encoded as a transgene in at least one of said locations, and characterised in that BY also encodes a polypeptide comprising the sushi domain of IL-15R alpha.

The present disclosure relates to a group B adenovirus encoding IL-15, pharmaceutical compositions comprising the same, use of the virus and/or compositions in treatment, in particular the treatment of cancer. The disclosure also extends to replication of the virus in a host cell and a process of formulating the virus.

### BACKGROUND

IL-15 is a cytokine that stimulates: CD8 positive, cytotoxic, T cells; NK cells and also NKT cells. It is thought to be critical for the division of T cells and the survival of memory T cells. It is also thought to be important for the activity/survival of NKT cells. There are some data to suggest that expression of IL-15 in the tumor microenvironment is an important factor which correlates with anti-tumor activity/responses. Thus, increasing the levels of IL-15 in the tumor microenvironment has become of interest in the treatment of cancer.

Perera et al (Proc Natl Acad Sci USA 2001, Apr 24; 98(9): 5146-5151) prepared a live vaccina virus encoding IL-15. Backhaus et al (Viruses 2019, 11, 914) discloses measles viruses encoding IL-12 or IL-15.

The present inventors prepared group B adenoviruses, particularly EnAd, encoding IL-15 and established that the level of cytokine expression was lower than desirable. Faced with this problem they set about optimising the viral constructs.

Surprisingly, expression is significantly improved when the group B adenovirus also encodes a polypeptide comprising at least the sushi domain from IL-15R alpha (including encoding the whole extracellular protein) linked or unlinked to the IL-15.

It can be difficult to measure levels of gene expression of transgenes encoded in oncolytic viruses. However, the present inventors have evidence to suggest that the transgenes encoded in viruses are not only expressed in the tumor microenvironment but the protein products can also be detected in the blood.

The tumor microenvironment is permissive to infiltration by adenoviruses according to the present disclosure, thereby allowing generous levels of IL-15 to be delivered to the desired location.

### SUMMARY OF THE DISCLOSURE

The present disclosure is summarised below in the following paragraphs:

1. A group B adenovirus comprising a sequence of formula (I):

5′ITR—B₁-B_{A}-B₂-B_{X}-B_{B}-B_{Y}-B₃-3′ITR (I)
wherein:
B₁ is a bond or comprises: E1A, E1B or E1A-E1B;
B_{A} comprises-E2B-L1-L2-L3-E2A-L4;
B₂ is a bond or comprises: E3;
B_{X} is a bond or a DNA sequence comprising: a restriction site, one or more transgenes or both;
B_{B} comprises L5;
B_{Y} comprises a sequence -G1-G2ₙ-G3ₘ-G4ₚ-G5_{q},
wherein G1 is a first transgene, G2 is a second transgene, G3 is a third transgene, G4 is a fourth transgene, G5 is a fifth transgene
B₃ is a bond or comprises: E4;
n is 0 or; m is 0 or; p is 0 or 1; q is 0 or 1;
wherein IL-15 is encoded in a transgene in position selected from G1, G2 G3, G4, G5 and combinations of two or three of the same, characterised in that By also encodes a polypeptide comprising the sushi domain of IL-15R alpha, (for example the sushi domain has a sequence shown in SEQ ID NO: 26).
   2. A group B adenovirus according to paragraph 1 wherein the polypeptide comprises a full-length IL-15R alpha extracellular domain, for example a full length IL-15R alpha including membrane anchored forms thereof (in one embodiment consists of or consists essentially of EC domain).
   3. A group B adenovirus according to paragraph 1 or 2, wherein the polypeptide encoding the sushi domain of IL-15R alpha comprises a transmembrane domain or GPI anchor, for example a transmembrane domain shown in SEQ ID NO: 28, 238, 239, 240, 241, 242.
   4. A group B adenovirus according to any one of paragraphs 1 to 3, wherein the polypeptide is linked to the IL-15.
   5. A group B adenovirus according to any one of paragraphs 1 to 4, wherein the encoded polypeptide is located in a different position to the IL-15 (i.e. is separate [unlinked] from IL-15).
   6. A group B adenovirus according to any one of paragraphs 1 to 5, wherein IL-15 is encoded in position G5.
   7. A group B adenovirus according to any one of paragraphs 1 to 6, wherein IL-15 is encoded a in position G4.
   8. A group B adenovirus according to any one of paragraphs 1 to 7, wherein IL-15 is encoded in position G3.
   9. A group B adenovirus according to any one of paragraphs 1 to 8, where in IL-15 is encoded in position G2.
   10. A group B adenovirus according to any one of paragraphs 1 to 9, wherein IL-15 is encoded in position G1. In one embodiment IL-15 is not encoded in position G1.
   11. A group B adenovirus according to any one of paragraphs 1 to 10, wherein the polypeptide comprising the IL-15R alpha sushi domain is encoded in G5.
   12. A group B adenovirus according to any one of paragraphs 1 to 11, wherein the polypeptide comprising the IL-15R alpha sushi domain is encoded in G4.
   13. A group B adenovirus according to any one of paragraphs 1 to 12, wherein the polypeptide comprising the IL-15R alpha sushi domain is encoded in G3.
   14. A group B adenovirus according to any one of paragraphs 1 to 13, wherein the polypeptide comprising the IL-15R alpha sushi domain is encoded in G2.
   15. A group B adenovirus according to any one of paragraphs 1 to 14, wherein the polypeptide comprising the IL-15R alpha sushi domain is encoded in G1.
   16. A group B adenovirus according to any one of paragraphs 1 to 15, wherein the virus also encodes IL-12, for example where the virus only encodes IL-12 once.
   17. A group B virus according to paragraph 16, wherein the IL-12 is encoded as a single chain fusion protein.
   18. A group B adenovirus according to paragraph 16 or 17, wherein the fusion protein is in the format p40-linker-p35.
   19. A group B adenovirus according to paragraph 18, where in the linker comprises one or more units of Gly₄Ser (G₄S), for example 1, 2, 3, 4, 5 or 6 units, such as 3 units of G4S.
   20. A group B adenovirus according to any one of paragraphs 17 to 19, wherein the IL-12 fusion protein comprises the sequence shown in SEQ ID NO: 115

IWELKKDVYVVELDWYPDAPGEMVVLTCDTPEEDGITWTL
DQSSEVLGSGKTLTIQVKEFGDAGQYTCHKGGEVLSHSLL
LLHKKEDGIWSTDILKDQKEPKNKTFLRCEAKNYSGRFTC
WWLTTISTDLTFSVKSSRGSSDPQGVTCGAATLSAERVRG
DNKEYEYSVECQEDSACPAAEESLPIEVMVDAVHKLKYEN
YTSSFFIRDIIKPDPPKNLQLKPLKNSRQVEVSWEYPDTW
STPHSYFSLTFCVQVQGKSKREKKDRVFTDKTSATVICRK
NASISVRAQDRYYSSSWSEWASVPCSGGGGSGGGGSGGG
GSRNLPVATPDPGMFPCLHHSQNLLRAVSNMLQKARQTL
EFYPCTSEEIDHEDITKDKTSTVEACLPLELTKNESCLNS
RETSFITNGSCLASRKTSFMMALCLSSIYEDLKMYQVEFK
TMNAKLLMDPKRQIFLDQNMLAVIDELMQALNFNSETVPQ
KSSLEEPDFYKTKIKLCILLHAFRIRAVTIDRVMSYLNAS.

21. A group B adenovirus according to any one of paragraphs 16 to 20 wherein the IL-12 is located in position G1, for example only encoded in position G1.
22. A group B adenovirus according to any one of paragraphs 16 to 21, wherein the IL-12 is located in position G2, for example only encoded in position G2.
23. A group B adenovirus according to any one of paragraphs 16 to 22, wherein the IL-12 is encoded in position G3, for example only encoded in position G3.
24. A group B adenovirus according to any one of paragraphs 16 to 23, wherein the IL-12 is encoded in position G4, for example only encoded in position G4. In one embodiment IL-12 is not encoded in position G4.
25. A group B adenovirus according to any one of paragraphs 16 to 24, wherein the IL-12 is encoded in G5, for example only encoded in position G5. In one embodiment IL-12 is not encoded in position G5.
26. A group B adenovirus according to any one of paragraphs 1 to 25, wherein n is 1.
27. A group B adenovirus according to any one of paragraphs 1 to 25, wherein n is 0.
28. A group B adenovirus according to any one of paragraphs 1 to 27, wherein m is 1.
29. A group B adenovirus according to any one of paragraphs 1 to 27, wherein m is 0.
30. A group B adenovirus according to any one of paragraphs 1 to 29, wherein p is 1.
31. A group B adenovirus according to any one of paragraphs 1 to 29, wherein p is 0.
32. A group B adenovirus according to any one of claims 1 to 31, wherein q is 1.
33. A group B adenovirus according to any one of claims 1 to 31 wherein q is 0.
34. A group B adenovirus according to any one of paragraphs 1 to 33, where at least one further cytokine is encoded in By, for example 2 or 3 cytokines are encoded.
35. A group B adenovirus according to paragraph 34, wherein the cytokine or cytokines is/are independently selected from: TNF super family (TNFSF) or TNF receptor superfamily (TNFRSF); TGF-beta superfamily (e.g. BMPs); Colony stimulating factor (CSF) family (e.g. GM-CSF, M-CSF); IL-1 family (e.g. IL-1, IL-18); Common cytokine receptor γ chain (γc) family (e.g. IL-2, IL-7, IL-21); IL-10 family (e.g. IL-24); IL-12 family (e.g. IL-23, IL-27); IL-17 family (e.g. IL-17E); Growth factor families (e.g. VEGF, FGF, IGF, PDGF, NGF, HGF, CTGF, TGF-alpha families); and interferon family (such as interferon type I, interferon type II and interferon type III).
36. A group B adenovirus according to paragraph 34 or 35, wherein the cytokine or cytokines are independently selected from: TNF-alpha, TNF-C, OX40L, CD154, FasL, LIGHT, TL1A, CD70, Siva, CD153, 4-1BB ligand, TRAIL, RANKL, TWEAK, APRIL, BAFF, CAMLG, NGF, BDNF, NT-3, NT-4, GITR ligand, EDA-A, EDA-A2, IFN-α, IFN-β, IFN-ε, IFN-γ, IFN-κ, and IFN-ω, Flt3 ligand, GM-CSF, M-CSF, VEGF-C, IL-1, IL-2, IL-7, IL-10, IL-15, IL-17, IL-18, IL-19, IL-20, IL-21, IL-22, IL-23, IL-24, IL-26, IL-27 IL-28, and IL-29.
37. A group B adenovirus according to any one of paragraphs 34 to 36, wherein the cytokine is IL-18.
38. A group B adenovirus according to any one of paragraphs 1 to 37, wherein By encodes interferon type I, such as interferon-a.
39. A group B adenovirus according to any one of paragraphs 34 to 38, wherein the cytokine is encoded in position G1, for example only encoded in position G1.
40. A group B adenovirus according to any one of paragraphs 34 to 39, wherein the cytokine is encoded in position G2, for example only encoded in G2.
41. A group B adenovirus according to any one of paragraphs 34 to 40, wherein the cytokine is encoded in position G3, for example only encoded in G3.
42. A group B adenovirus according to any one of paragraphs 34 to 41, wherein the cytokine is encoded in position G4, for example only encoded in G4.
43. A group B adenovirus according to any one of paragraphs 34 to 42, wherein the cytokine is encoded in position G5, for example only encoded in G5.
44. A group B adenovirus according to any one of paragraphs 1 to 43, wherein By encodes at least one chemokine, for example 1 chemokine.
45. A group B adenovirus according to paragraph 44, wherein the chemokine is selected from MIP-1 alpha, RANTES, IL-8 (CXCL8), CCL17, CCL19, CCL20, CCL21, CCL22, CXCL9, CXCL10, CXCL11, CXCL13, CXCL12 and CCL2.
46. A group B adenovirus according to paragraphs 44 or 45, wherein the chemokine is CXCL9.
47. A group B adenovirus according to any one of paragraphs 44 to 46, wherein the chemokine is CCL19.
48. A group B adenovirus according to any one of paragraphs 44 to 47, wherein the chemokine is CCL21.
49. A group B adenovirus according to any one of paragraphs 44 to 48, wherein the chemokine is encoded in G1, for example only encoded in G1.
50. A group B adenovirus according to any one of paragraphs 44 to 49, wherein the chemokine is encoded in G2, for example only encoded in G2.
51. A group B adenovirus according to any one of paragraphs 44 to 50, wherein the chemokine is encoded in G3, for example only encoded in G3.
52. A group B adenovirus according to any one of paragraphs 44 to 51, wherein the chemokine is encoded in position G4, for example only encoded in position G4.
53. A group B adenovirus according to any one of paragraphs 44 to 52, wherein the chemokine is encoded in position G5, for example only encoded in position G5.
54. A group B adenovirus according to any one of paragraphs 1 to 53, wherein the one or more transgenes in By are under the control of the major later promoter, for example all the transgenes are under the control of the major late promoter.
55. A group B adenovirus according to paragraph 54, wherein G1 is under the control of the major late promoter.
56. A group B adenovirus according to paragraph 54 or 55, wherein G2 is under the control of the major late promoter.
57. A group B adenovirus according to any one of paragraphs 54 to 56, wherein G3 is under the control of the major late promoter.
58. A group B adenovirus according to any one of paragraph 54 to 57, wherein G4 is under the control of the major late promoter.
59. A group B adenovirus according to any one of paragraphs 54 to 58, wherein G5 is under the control of the major late promoter.
60. A group B adenovirus according to any one of paragraphs 1 to 59, wherein the adenovirus is EnAd or Ad11, such as EnAd.
61. A group B adenovirus according to any one of paragraphs 1 to 60, wherein the adenovirus is replication capable, such as replication competent.
62. A group B adenovirus according to any one of paragraphs 1 to 61, wherein the adenovirus is oncolytic.
63. A composition comprising a group B adenovirus according to any one of paragraphs 1 to 62 and a pharmaceutically acceptable excipient, diluent or carrier.
64. A group B adenovirus according to any one of paragraphs 1 to 62 or a composition according to paragraph 63 for use in treatment, for example the treatment of cancer.
65. Use of a group B adenovirus according to any one of paragraphs 1 to 62 or a composition according to paragraph 63 in the manufacture of a medicament for the treatment of cancer.
66. A method of treating cancer comprising administering a therapeutically effective amount of an adenovirus according to any one of paragraphs 1 to 62 or a composition according to paragraph 63 to a subject in need thereof.

In one embodiment there is only one gene encoding IL-15 in the virus.

In one embodiment the leader sequence is employed for IL-15 is the non-native sequence, for example an Ig, CD33 or IL-2 leader sequence. This may optimise the expression.

In one embodiment there is only one gene encoding a polypeptide comprising the IL-15R alpha Sushi domain.

In one embodiment the Sushi domain comprises a transmembrane domain or GPI anchor, in particular a native transmembrane domain. In one embodiment the Sushi domain does not comprise a transmembrane domain or GPI anchor i.e. is not membrane anchored when expressed, in particular is expressed in a soluble form).

Soluble form as employed herein refers to a form that is unlinked, including free from attachment to a membrane and/or other protein. Thus, a soluble form includes a form that can be released, such as secreted from the cell.

In one embodiment the Sushi domain is unlinked, in particular it is not connected to the IL-15. In one embodiment the native leader sequence is employed for IL-15R alpha.

In one embodiment there is only one gene encoding IL-12.

Generally, G1, G2, G3, G4 and G5 represent transgenes, for example separated by suitable regulatory sequences, such as a polynucleotide encoding a 2A peptide.

The presently disclosed adenovirus may comprise 1, 2, 3, 4 or 5 transgenes at position By. Virtually any “gene” inserted in position By will be considered a transgene because it a non-natural location. Regulatory element are not considered to be “genes” in the context of the present specification.

The positions G1, G2, G3, G4 and G5 are nominal labels, which are defined relative to each other.

Thus, where there is only one transgene it will always be labelled G1. When there are two transgenes, they will usually be G1 and G2. When there are three transgenes, they will generally be labelled G1, G2 and G3. When there are four transgenes, they will generally be labelled G1, G2, G3 and G4. When there are five transgenes, they will generally be labelled G1, G2, G3, G4 and G5

In one embodiment B₁ comprises E1A-E1B.

In one embodiment B₂ comprises E3.

In one embodiment B_{X} is a bond.

In one embodiment B_{X}comprises one or more transgenes.

In one embodiment B₃ comprises E4.

In one embodiment one or more (such as all) the transgenes in position By are driven by an endogenous promoter, such as the major late promoter.

In one embodiment transgene expression, especially transgenes located in position By are NOT driven by an exogenous promoter.

In one embodiment the virus is replication deficient

In one embodiment the transmembrane domain comprises a sequence selected from the group comprising SEQ ID NO: 238, 239, 240, 241, 242, see sequence listing and Table 1 in the priority document.

In one embodiment the order of genes is disclosed in a Figure or an example herein. This order may be used as basis for an amendment to the claims. However, the key factor is the position of the IL-15 and/or sushi domain and/or IL-12 and/or IL-18. Thus, description of features of or more of these elements may be extracted from the Examples, “in isolation” if necessary.

In one independent aspect there is provided a novel virus, construct or component disclosed herein, for example in the sequence listing, pharmaceutical formulations comprising the same, use of the virus or construct or formulation comprising any one of the same in treatment, particularly in the treatment of cancer, such as a cancer disclosed herein.

In one embodiment the construct is a polynucleotide, such as a DNA construct, encoding a protein sequence listed in any one of SEQ ID NO: 36 to 73, 167 to 188 and 233 to 235.

In one embodiment the construct is a DNA cassette independently selected from SEQ ID NO: 116 to 156 and 189 to 210.

In one embodiment the virus according to the invention is independently selected from SEQ ID NO: 74 to 114 and 211 to 232.

In one embodiment the virus or construct is or relates to NG-796A, a composition comprising same or use thereof, particularly in therapy.

In a further independent aspect there is provided a virus encoding IL-15 without a gene encoding the Sushi domain.

In a further independent aspect there is provided a virus encoding IL-12 as per described herein, with or without a gene encoding IL-15.

In a further independent aspect there is provided a virus encoding IL-18 as per described herein, with or without a gene encoding IL-15.

The disclosure also relates to processes of preparing said viruses and compositions.

The viruses of the present disclosure are advantageous because they express adequate/good levels of IL-15 in vivo. In one embodiment two or more, such as 2, 3, 4 or 5 transgenes encoded by the virus are expressed well. The present inventors have also established that in some instances the relative location of the transgenes (i.e. ordering of the transgenes by reference to each other) in position By affects the stability of the virus and/or expression levels of the transgenes. In some instances, expression of a given transgene was extremely low, especially when the promoter is endogenous, which may reduce the therapeutic effectiveness as the local concentration of the polypeptide is reduced.

The virus life cycle is very complicated and not well understood, in particular there is a complex splicing mechanism. The latter may be affected by the precise local environment of the transgene and when problems arise, they are not easy to understand the solutions are not predictable. Thus, the present invention provides optimised viruses where problems with expression of individual transgenes, in particular IL-15, have been minimised.

### DETAILED DISCLOSURE

Group B adenovirus as employed herein refers to an adenovirus designated to group B including 3, 7, 11, 14, 16, 21, 34, 35, 51 and EnAd. The designation is generally assigned based on the viral capsid properties. Therefore, chimeric adenoviruses with capsids of a group B virus are designated to group B.

In one embodiment the adenoviruses of the present disclosure comprise a subgroup of B viruses, namely, Ad11, in particular Ad11p (the Slobitski strain) and derivatives thereof, such as EnAd.

In one embodiment the adenoviruses of the present disclosure are subgroup B viruses, namely, Ad11, in particular Ad11p (the Slobitski strain) and derivatives thereof, such as EnAd.

In one embodiment, the oncolytic virus has a fibre, hexon and penton proteins (such as all the capsid proteins) from the same serotype, for example Ad11, in particular Ad11p, for example found at positions 30812-31789, 18254-21100 and 13682-15367 of the genomic sequence of the latter wherein the nucleotide positions are relative to genbank ID 217307399 (accession number: GC689208 incorporated herein specifically by reference).

In one embodiment, the adenovirus is enadenotucirev (also known as EnAd and formerly as ColAd1). Enadenotucirev as employed herein refers to the chimeric adenovirus of disclosed as SEQ ID NO: 12 in WO2015/059303 incorporated herein by reference. It is a replication competent oncolytic chimeric adenovirus which has enhanced therapeutic properties compared to wild type adenoviruses (see WO2005/118825 incorporated herein by reference). EnAd has a chimeric E2B region characterised by DNA from Ad11p and Ad3, and deletions in E3/E4. The structural changes in enadenotucirev result in a genome that is approximately 3.5kb smaller than Ad11p thereby providing additional “space” for the insertion of transgenes. Almost all of the E3 region and part of the E4 region is deleted in EnAd. Therefore, it has significant space in the genome to accommodate additional genetic material whilst remaining viable. Furthermore, because EnAd is a subgroup B adenovirus, pre-existing immunity in humans is less common than, for example, Ad5. Other examples of chimeric oncolytic viruses with Ad11 fibre, penton and hexon include OvAd1 and OvAd2 (see WO2008/080003 incorporated by reference). Thus, in one embodiment the adenovirus employed is OvAd1 or OvAd2.

Oncolytic virus as employed herein refers to a virus with selectivity for cancer cells in that it preferentially kills cancer cells, for example because it preferentially infects cancer cells and/or the virus life cycle is dependent on a gene, such as p53 that is disregulated, for example over-expressed in cancer cells. The selectivity for cancer cells (therapeutic index) can be tested as described in WO2005/118825 incorporated herein by reference. In one embodiment the oncolytic virus preferentially infects cancer cells and goes on to replicate its genome and produce capsid proteins to generate new virus particles, for example as per EnAd.EnAd seems to preferentially infect tumour cells, replicates rapidly in these cells and causes cell lysis. This, in turn, can generate inflammatory immune responses thereby stimulating the body to fight the cancer. Part of the success of EnAd is hypothesised to be related to the fast replication of the virus in tumours in vivo.

IL-15 is a cytokine, which functions through interacting with a trimeric IL-15 receptor complex which includes the high affinity IL-15R alpha chain, and the common IL-15R beta and gamma chains. In one embodiment the IL-15 is human, for example as disclosed in Uniprot P40933 (incorporated herein specifically by reference) or SEQ ID NO: 23

IL-15R alpha is a subset of the IL-15 receptor complex. It has a 267 amino acid sequence including a 30 amino acid signal peptide. Thus, the mature protein is 237 amino acids in length. It may be provided as a soluble form (for example just the extracellular domain, such as amino acids 31-205) or as a membrane anchored form (for example including the transmembrane domain, such as amino acids 206 to 228 and optionally the cytoplasmic tail, such as amino acids 229 to 267). The domains of the mature protein include the Sushi domain at the N terminal, a linker region, Pro/Thr rich region (these three regions make up the extracellular domain); the transmembrane domain; and the cytoplasmic domain.

In the one embodiment the IL-15R alpha or fragment thereof according to the present disclosure is provided as in a soluble form, for example wherein there is no transmembrane domain and no cytoplasmic tail. This soluble form may be encoded as a separate protein from IL-15 or encoded such that the protein is linked to the IL-15 (for example linked as a fusion protein).

In one embodiment there is provided a membrane anchored form of IL-15R alpha or a fragment thereof as per the disclosure. Membrane anchored forms include a transmembrane domain or GPI anchor. In one embodiment the transmembrane domain is the native sequence, for example about amino acids 176 to 198 of the mature protein. In one embodiment the transmembrane domain is a non-native sequence (i.e. not the transmembrane domain from IL-15R alpha), for example selected from SEQ ID NO: 238, 239, 240, 241, 242.

The extracellular region of IL-15R alpha is approximately amino acids 1 to 175 of the mature protein.

Sushi domain as employed herein refers to the N-terminal domain located in the mature protein at amino acids approximately 1 to 65 (31 to 95 of the protein with the leader). This region is characterised by 2 disulphide bonds and, N and O-glycosylation sites.

The linker region in the mature protein is located at approximately amino acids 66 to 98.

The Pro/Thr rich region is approximately amino acids 99 to 175 of the mature protein and contains sites for O-glycosylation.

The transmembrane domain is located at approximately amino acids 176 to 198 of the mature protein.

The cytoplasmic domain is located at approximately amino acids 199 to 237 of the mature protein.

Thus, in one embodiment the IL-15R alpha comprises or consists of the Sushi domain, for example provided as a separate protein/polypeptide or linked to the IL-15.

In one embodiment the IL-15R alpha comprises or consists of the Sushi domain and the linker region, for example amino acids approximately 1 to 98 of the mature protein. In one embodiment this is encoded as a separate protein/polypeptide or linked to the IL-15.

In one embodiment the IL-15R alpha comprises or consists of the Sushi domain and the Pro/Thr rich region, for example amino acids 1 to 65 and 99 to 175 of the mature protein. In one embodiment this is encoded as a separate protein/polypeptide or linked to the IL-15.

In one embodiment the IL-15R alpha employed comprises or consists of the Sushi domain, linker domain and the Pro/Thr rich region, for example amino acids 1 to 175 of the mature protein. In one embodiment this is encoded as a separate protein/polypeptide or linked to the IL-15.

In one embodiment the IL-15R alpha comprises or consists of the Sushi domain, linker region, Pro/Thr rich region and the transmembrane region, for example amino acids 1 to 198 of the mature protein. In one embodiment this is encoded as a separate protein/polypeptide or linked to the IL-15.

Linked to the IL-15 as employed herein refers to linked via a linker (for example a G₄S linker or a linker disclosed starting on page 30 to 31 of WO2016/174200 SEQ ID NO: 26 to 90 and PPP therein and specifically incorporated by reference herein and may be used as basis for amending the claims: or linked directly via a bond, such as an amide bond. Thus, linked as employed herein generally refers to a genetic fusion protein.

Thus, unless the context indicates otherwise, linked as employed herein refers to the connection between “two” entities, for example such that the transgene is chimeric and thus appears as one gene encoded in the virus (such that there are regulatory elements separating the two nucleotide fragments) and also the expressed polypeptide in the mature form maintains the “connection”.

In one embodiment the Sushi domain and the IL-15 are linked, for example with a peptide bond or a peptide linker (e.g 1 to 20 amino acids in length), for example a linker disclosed herein or a G₄S linker (e.g. comprising 1, 2, 3, 4 or 5 units of G₄S).

In one embodiment the C-terminal of the Sushi domain is linked to the N-terminus of the IL-15. In one embodiment the C terminal of the IL-15 is linked to the N-terminus of the Sushi domain.

Unlinked as employed herein refers to where two units, such as the IL-15 and IL-15R alpha, are expressed as separate proteins/polypeptides. Thus, unlinked proteins will generally appear as separate transgenes encoded within the virus (such that there is a regulatory element separating the same, for example 2A peptide or the like) and the mature proteins expressed will also generally be separate entities i.e. NOT linked by a co-valent bond. However, separate proteins may assemble as complexes.

IL-12 as employed herein is a heterodimeric cytokine comprising p35 (encoded by IL-12A see Uniprot P29459) and p40 (encoded by IL-12B see Uniprot P29460). In one embodiment the p35 and p40 are unlinked. In one embodiment p35 and p40 are linked (for example by a linker, such as a linker disclosed herein, or linked by a bond, such as an amide bond).

The present disclosure also extends to employing variants of proteins and polypeptides disclosed herein wherein 1 to 10% (such as 1, 2, 3, 4, 5, 6, 7, 8, 9 or 10%) of the amino acids are changed or deleted, provided the desired function is retained.

Protein and polypeptide are generally used interchangeably, unless the context indicates otherwise. To the extent there is a distinction they proteins generally have tertiary structure and polypeptides may not have tertiary structure.

Generally, “comprising” in the context of polypeptides/proteins means, for example additional amino acids/fragments/polypeptides/protein can be appended provided the desired function is retained.

“Located in different positions”, as employed herein, refers to the fact that the transgenes are separated by a polynucleotide sequence. In adenoviruses genes can be located on different DNA strands because both strands of the virus are coding. Thus in one embodiment different positions refers to different DNA strands. However, in one embodiment the transgenes are in tandem, essentially encoded within the same strand. In one embodiment different locations are different regions of the virus, including, for example where the genes are in tandem. In one embodiment the genes are within the same region of the virus, including, (for example the genes are in tandem) and the “different position” refers to a sequence for example a regulatory element or transgene sequence that separates the “two positions”.

In one embodiment 0 transgenes separate the “different positions” and, for example a regulatory element (or elements) separates the two positions. In one embodiment 1 transgene separates the “different positions” (may include regulator elements associated therewith). In one embodiment 2 transgene separates the “different positions” (may include regulator elements associated therewith). In one embodiment 3 transgene separates the “different positions” (may include regulator elements associated therewith).

Transgene as employed herein refers to a gene that has been inserted into the genome sequence, which is a gene that is unnatural to the virus (exogenous) or not normally found in that particular location in the virus. Examples of transgenes are known in the art and discussed herein.

For example, the transgene may encode a protein, peptide, RNA molecule, such as an RNA molecule. Other examples of genetic material encoded by a transgene include for example antibodies or binding fragments thereof, chemokines, cytokines, immunmodulators, enzymes (for example capable of converting pro-drug in the active agent) and an RNAi molecule.

Transgene as employed herein also includes a functional fragment of the gene that is a portion of the gene which when inserted is suitable to perform the function or most of the function of the full-length gene.

Transgene and coding sequence are used interchangeably herein in the context of inserts into the viral genome, unless the context indicates otherwise. Coding sequence as employed herein means, for example a DNA sequence encoding a functional RNA, peptide, polypeptide or protein. Typically, the coding sequence is cDNA for the transgene that encodes the functional RNA, peptide, polypeptide or protein of interest Functional RNA, peptides, polypeptide and proteins of interest are described below.

Clearly the virus genome contains coding sequences of DNA. Endogenous (naturally occurring genes) in the genomic sequence of the virus are not considered a transgene, within the context of the present specification unless then have been modified by recombinant techniques such that they are in a non-natural location or in a non-natural environment.

In one embodiment transgene, as employed herein, refers to a segment of DNA containing a gene or cDNA sequence that has been isolated from one organism and is introduced into a different organism i.e. the virus of the present disclosure. In one embodiment, this non-native segment of DNA may retain the ability to produce functional RNA, peptide, polypeptide or protein.

Thus, in one embodiment the transgene inserted encodes a human or humanised protein, polypeptide or peptide.

Functions such as transcription, translation, etc require the gene (transgene) to be operably linked. Thus, generally a transgene or transgenes will be operably linked in the virus genome.

Operably linked as employed herein refers to transgenes being associated with the necessary regulatory elements to allow the genes to be functional i.e. to allow the genes to be “expressed”using the cellularly machinery once the virus is inside the cell.

In one or more embodiments, the transgene cassette is arranged as shown in the one or more of the Figures or the examples.

Transgene cassette as employed herein refers to a DNA sequence encoding one or more transgenes in the form of one or more coding sequences and one or more regulatory elements.

A transgene cassette may encode one or more monocistronic and/or polycistronic mRNA sequences.

In one embodiment, the transgene or transgene cassette encodes a monocistronic or polycistronic mRNA, and for example the cassette is suitable for insertion into the adenovirus genome at a location under the control of an endogenous promoter or exogenous promoter or a combination thereof. In particular the transgene cassette(s) is/are located in By under the control of an endogenous promoter, for example the major late promoter.

Monocistronic mRNA as employed herein refers to an mRNA molecule encoding a single functional RNA, peptide, polypeptide or protein.

In one embodiment, the transgene cassette encodes monocistronic mRNA.

In one embodiment the transgene cassette in the context of a cassette encoding monocistronic mRNA means a segment of DNA optionally containing an exogenous promoter (which is a regulatory sequence that will determine where and when the transgene is active) or a splice site (which is a regulatory sequence determining when a mRNA molecule will be cleaved by the spliceosome) a coding sequence (i.e. the transgene), usually derived from the cDNA encoding the protein/polypeptide of interest, optionally containing a polyA signal sequence and a terminator sequence.

In one embodiment, the transgene cassette may encode one or more polycistronic mRNA sequences.

Polycistronic mRNA as employed herein refers to an mRNA molecule encoding two or more functional RNA, peptides, polypeptide or proteins or a combination thereof. In one embodiment the transgene cassette encodes a polycistronic mRNA.

In one embodiment transgene cassette in the context of a cassette encoding polycistronic mRNA includes a segment of DNA optionally containing an exogenous promoter (which is a regulatory sequence that will determine where and when the transgene is active) or a splice site (which is a regulatory sequence determining when a mRNA molecule will be cleaved by the spliceosome) two or more coding sequences (i.e. the transgenes), usually derived from the cDNA for the protein, polypeptide or peptide of interest, for example wherein each coding sequence is separated by either an IRES or a high efficiency 2A peptide. Following the last coding sequence to be transcribed, the cassette may optionally contain a polyA sequence and a terminator sequence.

In one embodiment, the transgene cassette encodes a monocistronic mRNA followed by a polycistronic mRNA. In another embodiment the transgene cassette a polycistronic mRNA followed by a monocistronic mRNA.

The IL-15R alpha Sushi domain binds IL-15 with high affinity. Affinity can be measured by techniques such as BIAcore.

The Major Late Promoter (ML promoter or MLP) as employed herein refers to the adenovirus promoter that controls expression of the “late expressed” genes, such as the L5 gene. The MLP is a “sense strand” promoter. That is, the promoter influences genes that are downstream of the promoter in the 5′-3′ direction. The major late promoter as employed herein refers to the original major late promoter located in the virus genome.

### Structural Elements of Adenoviruses

As the structure of adenoviruses is, in general, similar the elements below are discussed in terms of the structural elements and the commonly used nomenclature referring thereto, which are known to the skilled person. When an element is referred to herein then we refer to the DNA sequence encoding the element or a DNA sequence encoding the same structural protein of the element in an adenovirus. The latter is relevant because of the redundancy of the DNA code. The viruses' preference for codon usage may need to be considered for optimised results.

Any structural element from an adenovirus employed in the viruses of the present disclosure may comprise or consist of the natural sequence or may have similarity over the given length of at least 95%, such as 96%, 97%, 98%, 99% or 100%. The original sequence may be modified to omit 10%, 9%, 8%, 7%, 6%, 5%, 4%, 3%, 2% or 1% of the genetic material. The skilled person is aware that when making changes the reading frames of the virus must not be disrupted such that the expression of structural proteins is disrupted.

In one embodiment the given element is a full-length sequence i.e. the full-length gene.

In one embodiment the given element is less than a full-length and retains the same or corresponding function as the full-length sequence.

In one embodiment for a given element which is optional in the constructs of the present disclosure, the DNA sequence may be less than a full-length and have no functionality.

The structural genes encoding structural or functional proteins of the adenovirus are generally linked by non-coding regions of DNA. Thus, there is some flexibility about where to “cut” the genomic sequence of the structural element of interest (especially non-coding regions thereof) for the purpose of inserting a transgene into the viruses of the present disclosure. Thus, for the purposes of the present specification, the element will be considered a structural element of reference to the extent that it is fit for purpose and does not encode extraneous material. Thus, if appropriate the gene will be associated with suitable non-coding regions, for example as found in the natural structure of the virus.

Thus, in one embodiment an insert, such as DNA encoding a restriction site and/or transgene, is inserted into a non-coding region of genomic virus DNA, such as an intron or intergenic sequence. Having said this some non-coding regions of adenovirus may have a function, for example in alternative splicing, transcription regulation or translation regulation, and this may need to be taken into consideration.

The sites identified herein, that are associated with the L5 region (for example between L5 and the E4 region), are suitable for accommodating a variety of DNA sequences encoding complex entities such as RNAi, cytokines, single chain or multimeric proteins, such as antibodies.

Gene as employed herein refers to coding and optionally any non-coding sequences associated therewith, for example introns and associated exons. In one embodiment a gene comprises or consists of only essential structural components, for example coding region, such as cDNA.

A discussion relating to specific structural elements of adenoviruses is provided in WO2016/174200 starting at page 33 line 8 to page 35 line 32, explicitly incorporated herein by reference and which may be employed as basis for amendment. If there are any doubts about what text is referred to then the priority document contains the same disclosure and will be employed to confirm the intentions of the drafter.

In one embodiment B_{X} comprises a buffer sequence. This sequence is an artificial non-coding sequence wherein a DNA sequence, for example comprising a transgene (or transgene cassette), a restriction site or a combination thereof may be inserted therein. This sequence is advantageous because it acts as a buffer in that allows some flexibility on the exact location of the transgene whilst minimising the disruptive effects on virus stability and viability.

The insert(s) can occur anywhere within a place corresponding to between positions 28192 bp and 28193 bp of the EnAd sequence disclosed in the prior art, such as WO2015/059303.

Thus, in one embodiment the restriction site or sites allow the DNA in the section to be cut specifically.

DNA sequence in relation to By as employed herein refers to the DNA sequence in the vicinity of the 3′ end of the L5 gene of B_{B}. In the vicinity of or proximal to the 3′ end of the L5 gene as employed herein refers to: adjacent (contiguous) to the 3′ end of the L5 gene or a non-coding region inherently associated therewith i.e. abutting or contiguous to the 3′ prime end of the L5 gene or a non-coding region inherently associated therewith (i.e. all or part of an non-coding sequence endogenous to L5). Alternatively, in the vicinity of or proximal to may refer to being close the L5 gene, such that there are no coding sequences between the By region and the 3′ end of the L5 gene.

Thus, in one embodiment By is joined directly to a base of L5 which represents the “end” of a non-coding sequence, or joined directly to a non-coding region naturally associated with L5.

Inherently and naturally are used interchangeably herein. In one embodiment B_{Y} comprises a buffer sequence. This sequence is advantageous because it acts as a buffer in that it allows some flexibility on the exact location of the transgene whilst minimising the disruptive effects on virus stability and viability.

E4 as employed herein refers to the DNA sequence encoding part or all of an adenovirus E4 region (i.e. polypeptide/protein region), which may be mutated such that the protein encoded by the E4 gene has conservative or non-conservative amino acid changes, and has the same function as wild-type (the corresponding non-mutated protein); increased function in comparison to wild-type protein; decreased function, such as no function in comparison to wild-type protein or has a new function in comparison to wild-type protein or a combination of the same as appropriate. In one embodiment the E4 region has E4orf4 deleted.

In one embodiment the E4 region is partially deleted, for example is 95%, 90%, 85%, 80%, 75%, 70%, 65%, 60%, 55%, 50%, 45%, 40%, 35%, 30%, 25%, 20%, 15%, 10% or 5% deleted. In one embodiment the E4 region has the sequence from 32188 bp to 29380 bp of the EnAd sequence disclosed in the prior art, such as WO2015/059303.

In one embodiment B₃ is a bond, i.e. wherein E4 is absent.

In one embodiment B₃ has the sequence consisting of from 32188 bp to 29380 bp of the EnAd sequence disclosed in the prior art, such as WO2015/059303.

As employed herein number ranges are inclusive of the end points.

The skilled person will appreciate that the elements in the formulas herein, such as formula (I) are contiguous and may embody non-coding DNA sequences as well as the genes and coding DNA sequences (structural features) mentioned herein. In one or more embodiments, the formulas of the present disclosure are attempting to describe a naturally occurring sequence in the adenovirus genome. In this context, it will be clear to the skilled person that the formula is referring to the major elements characterising the relevant section of genome and is not intended to be an exhaustive description of the genomic stretch of DNA.

E1A, E1B, E3 and E4 as employed herein each independently refer to the wild-type and equivalents thereof, mutated or partially deleted forms of each region as described herein, in particular a wild-type sequence from a known adenovirus.

“Insert” as employed herein refers to a DNA sequence that is incorporated either at the 5′ end, the 3′ end or within a given DNA sequence reference segment such that it interrupts the reference sequence. The latter is a reference sequence employed as a reference point relative to which the insert is located. An insert can, for example be either at least one restriction site insert, at least one transgene cassette or both. When the sequence is interrupted the virus will still comprise the original sequence, but generally it will be as two fragments sandwiching the insert.

In one embodiment the transgene or transgene cassette does not comprise a non-biased inserting transposon, such as a TN7 transposon or part thereof. Tn7 transposon as employed herein refers to a non-biased insertion transposon as described in WO2006/060314.

B_{X} and B_{Y} may independently comprise a restriction site, for example selected from NotI, FseI, AsiSI, SgfI and SbfI, in particular the restriction sites inserted are all different, such as sites specific for NotI and sites specific for FseI located in B_{X} and SgfI and SbfI located in By.

As discussed above in one embodiment the region B_{X} and/or B_{Y} do not comprise a restriction site. The viruses and constructs of the present disclosure can be prepared without restriction sites, for example using synthetic techniques. These techniques allow a great flexibility in the creation of the viruses and constructs. Furthermore, the present inventors have established that the properties of the viruses and constructs are not diminished when they are prepared by synthetic techniques.

Regulatory sequences are disclosed in WO2016/174200 page 39 starting at line 15 to page 41 line 13, specifically incorporated explicitly herein by reference and which may be employed as a basis for amendment.

“High self-cleavage efficiency 2A peptide” or “2A peptide” as employed herein refers to a dividing sequence in a single polypeptide that facilitates the generation of multiple individual separate polypeptides. Suitable 2A peptides include P2A, F2A, E2A and T2A. The present inventors have noted that once a specific DNA sequence encoding a given 2A peptide is used once, the same specific DNA sequence may not be used a second time. However, redundancy in the DNA code may be utilised to generate a DNA sequence that is translated into the same 2A peptide. Thus, using 2A peptides is particularly useful when the cassette encodes polycistronic mRNA because it results in the expression of multiple individual proteins or peptides.

In one embodiment the encoded P2A peptide employed has the amino acid sequence of SEQ ID NO: 4. In one embodiment the encoded T2A peptide employed has the amino acid sequence of SEQ ID NO: 5. In one embodiment the encoded E2A peptide employed has the amino acid sequence of SEQ ID NO: 6. In one embodiment the encoded F2A peptide employed has the amino acid sequence of SEQ ID NO: 7.

In one embodiment the regulator of gene expression is a splice acceptor sequence, for example as disclosed herein.

### Formulations

The present disclosure relates also extends to a pharmaceutical formulation of a virus as described herein.

In one embodiment there is provided a liquid parenteral formulation, for example for infusion or injection, of a replication capable oncolytic according to the present disclosure wherein the formulation provides a dose in the range of 1×10¹⁰ to 1×10¹⁴ viral particles per volume of dose.

Parenteral formulation means a formulation designed not to be delivered through the GI tract. Typical parenteral delivery routes include injection, implantation or infusion. In one embodiment the formulation is provided in a form for bolus delivery.

In one embodiment the parenteral formulation is in the form of an injection. Injection includes intravenous, subcutaneous, intra-tumoural or intramuscular injection. Injection as employed herein means the insertion of liquid into the body via a syringe. In one embodiment, the method of the present disclosure does not involve intra-tumoural injection.

In one embodiment the parenteral formulation is in the form of an infusion.

Infusion as employed herein means the administration of fluids at a slower rate by drip, infusion pump, syringe driver or equivalent device. In one embodiment, the infusion is administered over a period in the range of 1.5 minutes to 120 minutes, such as about 3, 4, 5, 6, 7, 8, 9, 10, 11, 12, 13, 14 15, 16, 17, 18, 19 20, 25, 30, 35, 40, 45, 50, 55, 60, 65, 70, 65, 80, 85, 90, 95, 100, 105, 110 or 115 minutes.

In one embodiment one dose of the formulation less than 100mls, for example 30mls, such as administered by a syringe driver. In one embodiment one dose of the formulation is less than 10 mls, for example 9, 8, 7, 6, 5, 4, 3, 2 or 1 mls. In one embodiment one dose of the formulation is less than 1 ml, such as 0.9, 0.8, 0.7, 0.6, 0.5, 0.4, 0.3, 0.2 or 0.1 mls.

In one embodiment, the injection is administered as a slow injection, for example over a period of 1.5 to 30 minutes.

In one embodiment, the formulation is for intravenous (i.v.) administration. This route is particularly effective for delivery of oncolytic virus because it allows rapid access to the majority of the organs and tissue and is particular useful for the treatment of metastases, for example established metastases especially those located in highly vascularised regions such as the liver and lungs.

Therapeutic formulations typically will be sterile and stable under the conditions of manufacture and storage. The composition can be formulated as a solution, microemulsion, liposome, or other parenteral formulation suitable for administration to a human and may be formulated as a pre-filled device such as a syringe or vial, particular as a single dose.

The formulation will generally comprise a pharmaceutically acceptable diluent or carrier, for example a non-toxic, isotonic carrier that is compatible with the virus, and in which the virus is stable for the requisite period of time.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a dispersant or surfactant such as lecithin or a non-ionic surfactant such as polysorbate 80 or 40. In dispersions the maintenance of the required particle size may be assisted by the presence of a surfactant. Examples of isotonic agents include sugars, polyalcohols such as mannitol, sorbitol, or sodium chloride in the composition.

In one embodiment, parenteral formulations employed may comprise one or more of the following a buffer, for example 4-(2-hydroxyethyl)-1-piperazineethanesulfonic acid, a phosphate buffer and/or a Tris buffer, a sugar for example dextrose, mannose, sucrose or similar, a salt such as sodium chloride, magnesium chloride or potassium chloride, a detergent such as a non-ionic surfactant such as briji, PS-80, PS-40 or similar. The formulation may also comprise a preservative such as EDTA or ethanol or a combination of EDTA and ethanol, which are thought to prevent one or more pathways of possible degradation.

In one embodiment, the formulation will comprise purified adenovirus according to the present disclosure, for example 1×10¹⁰ to 1×10¹⁴ viral particles per dose, such as 1×10¹⁰ to 1×10¹² viral particles per dose. In one embodiment the concentration of virus in the formulation is in the range 2×10⁸ to 2×10¹⁴ vp/mL, such as 2×10¹² vp/ml.

In one embodiment, the parenteral formulation comprises glycerol.

In one embodiment, the formulation comprises an adenovirus as described herein, HEPES (N-2-hydroxyethylpiperazine-N′-2-ethanesulfonic acid), glycerol and buffer.

In one embodiment, the parenteral formulation consists of virus of the disclosure, HEPES for example 5 mM, glycerol for example 5-20% (v/v), hydrochloric acid, for example to adjust the pH into the range 7-8 and water for injection.

In one embodiment 0.7 mL of virus of the disclosure at a concentration of 2×10¹² vp/mL is formulated in 5 mM HEPES, 20% glycerol with a final pH of 7.8.

In one embodiment a virus of the present disclosure is formulated as a liquid formulation, comprising:
a) 15 to 25% v/v glycerol, for example 16, 17, 18, 19, 20, 21% v/v glycerol; and
b) 0.1 to 1.5% v/v ethanol, for example 0.2-1%, such as 1% v/v ethanol;
c) a buffer,

wherein the pH of the formulation is in the range 8.0 to 9.6, for example 8.0, 8.1, 8.2, 8.3, 8.4, 8.5, 8.6, 8.7, 8.8, 8.9, 9.0, 9.1, 9.2, 9.3, 9,4 or 9.5, see for example WO2019/149829 incorporated herein by reference.

The formulation may further comprises a surfactant, for example polysorbate 20, 40, 60, or 80, such as 0.05-0.15% polysorbate 20, 40, 60, or 80, such as 0.05-0.15% polysorbate 80, such as 0.115% polysorbate 80.

In one embodiment the formulation further comprises methionine, for example 0.01-0.3 mM, for example 0.1 to 0.3, such as 0.25 mM methionine.

In one embodiment the formulation further comprises arginine, for example 5 to 20 mM, such as 15 mM arginine.

In one embodiment the buffer comprises meglumine.

Thus, in one specific embodiment the liquid formulation comprises:
a) 15-20% v/v glycerol;
b) 1-1.5% v/v ethanol;
c) 0.1-0.2% v/v polysorbate 80;
d) 0.2-0.3 mM methionine;
e) 10-20 mM arginine; and
f) a buffer, such as meglumine;
wherein the pH of the formulation is at a pH in the range 8.0 to 9.6, such as pH 8.

A thorough discussion of pharmaceutically acceptable carriers is available in Remington's Pharmaceutical Sciences (Mack Publishing Company, N.J. 1991).

In one embodiment, the formulation is provided as a formulation for topical administrations including inhalation.

Suitable inhalable preparations include inhalable powders, metering aerosols containing propellant gases or inhalable solutions free from propellant gases. Inhalable powders according to the disclosure will generally contain an adenovirus as described herein with a physiologically acceptable excipient.

### Treatment

In a further aspect, the present disclosure extends to a adenovirus or a formulation thereof as described herein for use in treatment, in particular for the treatment of cancer.

In one embodiment, the method of treatment is for use in the treatment of a tumour, in particular a solid tumour.

Tumour as employed herein is intended to refer to an abnormal mass of tissue that results from excessive cell division that is uncontrolled and progressive, also called a neoplasm. Tumours may be either benign (not cancerous) or malignant. Tumour encompasses all forms of cancer and metastases. In one embodiment the tumour is not benign.

In one embodiment, the tumour is a solid tumour. The solid tumour may be localised or metastasised.

In one embodiment, the tumour is of epithelial origin.

In one embodiment, the tumour is a malignancy, such as colorectal cancer, hepatoma, prostate cancer, pancreatic cancer, breast cancer, ovarian cancer, thyroid cancer, renal cancer, bladder cancer, head and neck cancer or lung cancer.

In one embodiment, the tumour is a colorectal malignancy.

Malignancy as employed herein means cancerous cells.

In one embodiment, the adenovirus is employed in the treatment or prevention of metastasis.

In one embodiment, the method or formulation herein is employed in the treatment of drug resistant cancers.

In one embodiment, the adenovirus is administered in combination with the administration of a further treatment or therapy, in particular a further cancer treatment or therapy.

In one embodiment, there is provided a virus or formulation according to the present disclosure for use in the manufacture of a medicament for the treatment of cancer, for example a cancer described above.

In a further aspect, there is provide a method of treating cancer comprising administering a therapeutically effective amount of a virus or formulation according to the present disclosure to a patient in need thereof, for example a human patient.

In one embodiment, the oncolytic virus or formulation herein is administered in combination with another therapy.

“In combination” as employed herein is intended to encompass where the oncolytic virus is administered before, concurrently and/or post cancer treatment or therapy.

Cancer therapy includes surgery, radiation therapy, targeted therapy and/or chemotherapy.

Cancer treatment as employed herein refers to treatment with a therapeutic compound or biological agent, for example an antibody intended to treat the cancer and/or maintenance therapy thereof.

In one embodiment, the cancer treatment is selected from any other anti-cancer therapy including a chemotherapeutic agent, a targeted anticancer agent, radiotherapy, radio-isotope therapy, a biological therapeutic, an immunotherapy (such as checkpoint inhibitors of the PD1 signaling pathway, including pembrolizumab, nivolumab, cemiplimab, atezolizumab, avelumab, durvalumab) a further oncolytic virus, a cellular therapy (such as a chimeric antigen receptor cellular therapy) or any combination thereof.

Thus, in one embodiment the combination therapy comprises a PD-1 inhibitor, for example pembrolizumab, nivolumab, cemiplimab, JTX-4014 (Jounce Therapeutics), spartalizumab, camrelizumab, sintilimab, tislelizumab, toripalimab, dostarlimab, INCMGA00012 (macrogenics), AMP-224 (AstraZeneca/MedImmune and GSK) and AMP-514.

In one embodiment the combination comprises a PD-L1 inhibitor atezolizumab, avelumab, durvalumab, KN035, CK-301 (Checkpoint Therapeutics), AUNP12 (Pierre Fabre), CA-170 and BMS-986189.

In one embodiment, the virus of the present disclosure or a formulation thereof may be used as a pre-treatment to the therapy, such as a surgery (neoadjuvant therapy), for example to shrink the tumour, to treat metastasis and/or prevent metastasis or further metastasis. The oncolytic adenovirus may be used after the therapy, such as a surgery (adjuvant therapy), for example to treat metastasis and/or prevent metastasis or further metastasis.

Concurrently as employed herein is the administration of the additional cancer treatment at the same time or approximately the same time as the oncolytic adenovirus formulation. The treatment may be contained within the same formulation or administered as a separate formulation.

In one embodiment, the virus is administered in combination with the administration of a chemotherapeutic agent.

Chemotherapeutic agent as employed herein is intended to refer to specific antineoplastic chemical agents or drugs that are selectively destructive to malignant cells and tissues. For example, alkylating agents, antimetabolites, anthracyclines, plant alkaloids, topoisomerase inhibitors, and other antitumour agents. Other examples of chemotherapy include doxorubicin, 5-fluorouracil (5-FU), paclitaxel, capecitabine, irinotecan, and platins such as cisplatin and oxaliplatin (including combinations of two or more of the same). The preferred dose may be chosen by the practitioner based on the nature of the cancer being treated.

In one embodiment the therapeutic agent is ganciclovir, which may assist in controlling immune responses and/or tumour vascularisation.

In one embodiment one or more therapies employed in the method herein are metronomic, that is a continuous or frequent treatment with low doses of anticancer drugs, often given concomitant with other methods of therapy.

Subgroup B oncolytic adenoviruses, in particular Ad11 and those derived therefrom such as EnAd may be particularly synergistic with chemotherapeutics. Moreover, the immunosuppression that occurs during chemotherapy may allow the oncolytic virus to function with greater efficiency.

In one embodiment the virus according to the present disclosure or formulation thereof is employed in combination with a cellular therapy, for example a T cell therapy, an NKT cell therapy, NK cell therapy or macrophage cell therapy including transgenic forms thereof (such as chimeric antigen receptor cells, in particular CAR-T cells and CAR-NKT cells).

Transgenic cells as employed herein refer to engineered cells, for example engineered using recombinant techniques to include non-native polynucleotide(s) that modify the function of the cell i.e the cell is modified to express a synthetic receptor on its surface.

CAR as employed herein refers to chimeric antigen receptor i.e a synthetic receptor, such as an antibody binding domain coupled to signalling function, such as an intracellular signalling function. CARs are most commonly created by joining heavy and light chain variable regions from a monoclonal antibody. The receptors bind antigen or ligand to which they are specific and stimulate signalling pathways in the transgenic cell.

First generation CAR-T cells often had intracellular signalling unit based on CD3-zeta. However, second generation CARs generally have costimulatory element, such as CD28 and 4-1B_{B}, CD136, CD137 or CD27 and ICOS built into the intracellular signalling domain (see FIG. 14 herein, for example see U.S. Pat. No. 7,446,190, Dotti et al 2009 (Human Gene Therapy 20: 1229-1239 (November 2009). Finney et al J Immuno. 1998, Sep 15; 161(6): 2791-2797. Finney et al 2004 J Immunol Jan 1, 172(1) 104-113. Milone et al Mol Ther. 2009 August; 17(8): 1453-1464.

Companies such as ProMab Biotechnologies, Inc make these products commercially available.

Thus, in one embodiment the CAR comprises a CD3 zeta signalling unit. The following disclose first generation CARs: Irving and Weiss, Cell 1991 Mar 8; 64(5): 891-901. Letourmeur 1991 Oct 15; 88(20) 8905-8909. Romeo, Cell, Vol 68, issue 5, p889-897, Mar. 6, 1992.*

In one embodiment the CAR comprises a CD28 signalling unit, see for example Maher et al, Nat Biotechnol 2002 Jan; 20(1); 70-75 and Carpenito et al PNAS Mar. 3, 2009 106(9) 3360-3365.*

In one embodiment the CAR comprises a CD27 signalling unit. The later makes an essential contribution to mature CD4+ and CD8+ T cell function.

In one embodiment the CAR comprises an ICOS signalling unit, wherein ICOS stands for inducible T-cell co-stimulator.

In one embodiment the CAR comprises 4 1B_{B}, see for example Imai 2004, Leukemia 18, 676-684.*

In one embodiment the CAR therapy comprises one co-stimulatory factor.

In one embodiment the CAR therapy comprises a combination of co-stimulatory factors, for example 2, 3, or 4, such as CD28 and 4-1B_{B}, CD28 and ICOS, CD27 and 4-1B_{B} or CD27 and ICOS.

Guedan et al Blood 2014, 124(7): 1070-1080, incorporated herein by reference, discloses ICOS based chimeric antigen receptors.* Duong PLoS 2013 May 7;8(5) discloses engineering T cell function using chimeric antigen receptors.*

Signalling unit as employed herein is element that contribute to the cellular signalling of the CAR.

Chimeric T cell receptors are disclosed in US2004043401.*

* The construction features (signalling aspects not specificity) of the CAR disclosed here are incorporated by reference and may be used as the basis for an amendment to the claims.

The binding domain of the CAR is similar to an antibody and may, for example comprise a scFv, see for example Kuwana et al Biochem Biophys Res Commun. 1987 Dec 31, 149(3); and Eshhar et al Proc Natl Acad Sci USA 1993 Jan 15; 90(2):270-724.* Second generation CARS

In one embodiment the binding domain of the CAR is specific to a blood antigen, for example CD19, CD30, CD123, FLT, (including combinations such as CD19 and CD20 or CD22) in particular useful in the treatment of a hematological cancer, such as ALL, AML, CLL, DLBCL, BCMA, leukemia and multiple myeloma. Porter et al N Engl J Med 2011; 365: 1937-1939 discloses CAR modified T cells in CLL. Grupp et al N Engl J Med 2013 April 18; 368 (16) 1509-1518 discloses CAR modified T cells for ALL. Maude et al N Engl J Med 2014, 371: 1507-1517 disclosed CAR-T cells for sustained remission of Leukemia. Garfall et al N Engl J Med 2015; 373: 1040-1047 disclosed CART cells against CD19 for multiple myeloma.

In one embodiment the CAR is specific to a cancer antigen.

Cancer antigens (also referred to as tumor antigens) are antigens found specifically on cancer cells (i.e. generally not found on healthy cells or highly upregulated on cancer cells) including for example CEA, MUC-1, EpCAM, HER receptors HER1, HER2, HER3, HER4, PEM, A33, G250, carbohydrate antigens Le^{y}, Le^{x}, Le^{b}, PSMA, TAG-72, STEAP1, CD166, CD24, CD44, E-cadherin, SPARC, ErbB2, ErbB3, WT1, MUC1, LMP2, idiotype, HPV E6&E7, EGFRvIII, HER-2/neu, MAGE A3, p53 nonmutant, p53 mutant, NY-ESO-1, GD2, PSMA, PCSA, PSA, MelanA/MART1, Ras mutant, proteinase3 (PR1), bcr-abl, tyrosinase, survivin, PSA, hTERT, particularly WT1, MUC1, HER-2/neu, NY-ESO-1, survivin and hTERT. In one embodiment the CAR binding domain is specific to a cancer antigen. Can

In one embodiment the CAR binding domain targets aberrant sugars on the surface of cancer cells.

In one embodiment the CAR is specific to a stromal antigen.

Stromal antigens as employed herein are antigens only expressed on stromal cells, for example antigens expressed on cancer cells and stromal cells are considered to be cancer antigens in the context of the present specification. Examples of stromal antigens include CD163, CD206, CD68, CD11c, CD11b, CD14, CSF1 receptor, CD15, CD33 and CD66b.

In one embodiment the CAR binding domain is specific to an antigen selected from the group comprising HER-3, HER-4, CEA, EGFRviii, PSMA, CD20, VEGFR-1, VEGFR-3, c-Met, Lewis A, ROR-1, CD326, CD133, NKG2d, MUC-1, PSCA, PSA, CA-125, Notch and FLT-3.

In one embodiment the engineered cell encodes at least two entities, for example CD19 CAR and PD-1 siRNA, CD19 TIGIT siRNA, BCMA-CS1, or BCMA-CD33.

Thus, in on one embodiment the CAR is specific to:
CD19 (for example CD19-CD28, CD19scFv-CD28-CD3ζ, CD19scFv-4-1BB-CD3ζ, CD19scfv-CD28-4-1BB, CD19scFv-CD28-4-1BB-CD3ζ, or iCas9-T2A-antiCD19scFv-CD28-CD3ζ, CD19FLAG-CD28-CD3ζ or iCas9 HA-T2A-antiCD19scFv-CD28-CD3ζ-GGGS-FLAG, humanised CD19 scFv-TM28-CD28-CD3ζ, CD19 scFv-Beam-TM28-CD28-CD3ζ or humanised CD19 scFv-Beam-TM28-CD28-CD3ζ, CD19 scFv-CD22 scFv-4-1BB-CD3-T2A-tEGFR or, CD19 scFv-TM28-GITR-CD3ζ or CD19 scFv-TM8-GITR-CD3ζ);
Mesothelin (for example mesothelin scFv-CD28-CD3ζ, mesothelin scFv-4-1B_{B}-CD3ζ, mesothelin scFv-CD28-4-1BB-CD3ζ, mesothelin scFv FLAG-4-1BB-CD3ζ, mesothelin scFV-TM28-CD28-4-1BB-CD3ζ, mesothelin scFv-Beam-TM28-4-1BB-CD3ζ, mesothelin scFv-Beam-CD28-CD3ζ, mesothelin scFv-TM8-4-1BB-CD3ζ, mesothelin scFv-TM28-CD28-CD3ζ);
VGFR2 (for example VGFR2 scFv-CD28 CD3ζ);
GPC3 (for example GPC3 scFv-CD28-CD3ζ)
CD133 (CD133 scFv-CD28-CD3ζ);
EpCAM (for example EpCAM scFv-CD28-CD3ζ such as a version where Nhel restriction site introduced, N-terminal of scFv amino acid);
EGFR (for example EGFR scFv-CD28-CD3ζ, EGFR scFv-4-1BB-CD3ζ, EGFR scFv-TM28-GITR-CD3ζ, scFv-TM28-CD3ζ-GITR;
CD33 (for example CD33 scFv-TM28-CD28-CD3ζ or CD33 scFv-Beam 2-TM28-CD28-CD3ζ);
CD38 (for example CD38 scFv-TM28-CD28-CD3ζ);
CD138 (for example CD138 scFv-Beam-TM28-CD28-CD3ζ)
CD22 (for example CD22 scFv-TM28-CD28 CD3ζ, -CD22 scFv-TM28-4-1BB CD3ζ or CD22 scFV-Beam-TM28-CD28-CD3ζ)
BCMA (for example BCMA-4-CD28 CD3ζ or humanized, BCMA-4 scFv-TM8-4-1BB-CD3ζ or BCMA-2 scFv-Tm-CD28-CD3ζ)
HER2 (for example HER2 scFv-CD28-CD3ζ, HER2 scFv-4-1-BB-CD3Z-EGFRt or HER scFV-4-1BB-CD3ζ-GFP)
CD4 (for example CD4 scFv-Beam-TM28-CD28-CD3ζ)
ROR-1 (for example ROR-1 scFv TM28-CD28-CD3ζ, ROR-1 scFv TM28-4-1BB-CD3ζ or humanised ROR-1 scFv TM28-4-1BB-CD3ζ)
CD19&CD22 (for example CD19 scFv CD22 scFv-4-1BB-CD3ζ or CD19 scFv-CD22 scFv-4-1BB-CD3-T2A-RQR8)
CEA (for example CEA scFv-TM28-CD28 CD3ζ or humanised CEA scFv -TM28-CD28 CD3ζ
NGFR (for example NGFR scFv-TM28-CD28-CD3ζ)
MCAM (for example MCAM scFv-TM28-CD28-CD3ζ)
CD47 (for example CD47 scFv-TM28-Cd28-CD3ζ or humanised CD47 scFv-TM28-CD28-CD3ζ)
PDL-1(for example PDL-1 scFV-TM28-CD28-CD3ζ)
CD123 (for example CD123 scFv-TM28-CD28-CD3ζ)
CD37 (for example CD37 scFv-TM28-CD28-CD3ζ, CD37 scFv-TM28-4-1-BB-CD3ζ
CS1 (for example CS1 scFv-TM28-CD28-CD3ζ)
B7H4 (for example B7H4 scFv-TM28-CD28-CD3ζ)
CD24 (for example CD24 scFv-TM28-CD28-CD3ζ), and
CD20 (for example CD20 scFv-TM28-CD28-CD3ζ)
NKG2D such as CYAD-01,
FLT3, such as AMG 553
DLL3.

In one embodiment the CAR is specific to HER-2, for example with a specificity of the CAR employed in the Examples disclosed herein.

In one embodiment the CAR is provided in a T cell (such as autologous T cells or allogenic T cells, more specifically HLA matched T cells). In one embodiment the CAR-T cell is selected from tisagenlecleucel, axicabtagene ciloleucel, lisocabtagene maraleucel, idecabtagene vicleucel, brexucabtagene autoleucel, JCAR015 (CD19 CAR T from Juno), Descartes-08 (BCMA) and AMG119.

In one embodiment the immune cells is a phagocytic cell, for example encoding a CAR listed herein, such as CD19 scFv-label-CAR or mesothelin scFv CAR. In one embodiment the phagocytic cell is a macrophage, such as a THP 1 cell),

In one embodiment the CAR is provided in an NKT cell. The benefit of NKT cell cars, is that they do not require HLA matching with the patient Thus, they can be employed to provide an “off the shelf product”, for example with the specificity listed herein. WO2013/040371 discloses NKT engineered with a CAR and incorporated herein by reference. In one embodiment the NKT cell enc

In one embodiment the immune cell is an NK cell, see for example Tran et al, J Immunol 1995 Jul, 155(2); 1000-1009 incorporated herein by reference.

Thus, in one embodiment the immune cell therapy further comprises a transgene (i.e. an engineered gene) encoding a cytokine, for example selected from IL-2, IL-5, IL-7, IL-12 and IL-15.

Immune cells such as T cells, NKT cells may be activated or have activity sustained by the IL-15 expressed by the virus of the present invention. This may help counteract the anergic/hypoxic microenvironment of tumour. The latter may have the ability to neutralise the killing power of the native cells and even the engineered cellular therapy employed in combination with the present invention. Thus, use of the virus of the present disclosure may trigger several mechanisms for killing cancer, especially when used in combination with cellular therapy.

Therapeutic dose as employed herein refers to the amount of virus, such as oncolytic adenovirus that is suitable for achieving the intended therapeutic effect when employed in a suitable treatment regimen, for example ameliorates symptoms or conditions of a disease. A dose may be considered a therapeutic dose in the treatment of cancer or metastases when the number of viral particles may be sufficient to result in the following: tumour or metastatic growth is slowed or stopped, or the tumour or metastasis is found to shrink in size, and/or the life span of the patient is extended. Infection of cancer cells after systemic delivery of the viruses of the present disclosure is an indication of a therapeutic dose i.e. it has been delivered to the target cells. Suitable therapeutic doses are generally a balance between therapeutic effect and tolerable toxicity, for example where the side-effect and toxicity are tolerable given the benefit achieved by the therapy.

In one embodiment, a virus or therapeutic construct according to the present disclosure (including a formulation comprising same) is administered weekly, for example one week 1 the dose is administered on day 1, 3, 5, for example followed by one dose each subsequent week or multiple doses in the second week.

In one embodiment, a virus or therapeutic construct according to the present disclosure (including a formulation comprising same) is administered bi-weekly or tri-weekly, for example is administered in week 1 one on days 1, 3 and 5, and on week 2 or 3 is also administered on days 1, 3 and 5 thereof. This dosing regimen may be repeated as many times as appropriate.

In one embodiment the first dose is lower than the subsequent doses, for example the first dose is in the range 1×10¹⁰ to 1×10¹² viral particles and the subsequent doses are in the range 1×10¹¹ to 1×10¹³ viral particles.

In one embodiment 6 doses are given over a two week period, for example day 1, 3, 5, 8, 10 and 12, such as where each dose may be given+/−1 day, including where the dose on day 1 is lower than the other doses.

In one embodiment, a virus or therapeutic construct according to the present disclosure (including a formulation comprising same) is administered monthly.

In one embodiment, the viruses and constructs of the present disclosure are prepared by recombinant techniques. The skilled person will appreciate that the armed adenovirus genome can be manufactured by other technical means, including entirely synthesising the genome or a plasmid comprising part of all of the genome. The skilled person will appreciate that in the event of synthesising the genome the region of insertion may not comprise the restriction site nucleotides as the latter are artefacts following insertion of genes using cloning methods.

The disclosure herein further extends to an adenovirus of formula (I) or a subformula thereof, obtained or obtainable from inserting a transgene or transgene cassette.

“Is” as employed herein means comprising.

In the context of this specification “comprising” is to be interpreted as “including”.

Embodiments of the invention comprising certain features/elements are also intended to extend to alternative embodiments “consisting” or “consisting essentially” of the relevant elements/features.

Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section may be used as basis for an amendment.

The present application claims priority from GB2102049.0 filed 13 Feb. 2021, incorporated herein by reference especially the sequences, which are explicitly incorporated herein. The priority document may be employed to correct errors in the present specification.

The present invention is further described by way of illustration only in the following examples, which refer to the accompanying Figures, in which:

### DESCRIPTION OF THE FIGURES

FIG. 1A shows effect of different combinations of recombinant IL-12, IL15 and IL-18 proteins on IFNg production by cultures of primary breast (T63), colorectal (T64) and kidney (T65) tumour cell preparations.

FIG. 1B shows expression of the CD25 activation marker on CD4 and CD8 T-cells and NK cells from cultures of primary breast tumour (60) cell preparations treated with different combinations of IL-12, IL15 and IL-18.

FIG. 1C shows expression of the CD107a marker of activated degranulation on CD4 and CD8 T-cells and NK cells from cultures of primary breast tumour (60) cell preparations treated with different combinations of IL-12, IL15 and IL-18.

FIG. 1D shows intracellular IFNg expression in CD4 and CD8 T-cells and NK cells from PBMCs treated with different combinations of IL-12, IL15 and IL-18.

FIG. 2A shows kinetic analysis of PBMC-derived T-cell mediated killing (apoptosis induction) of target cell fibroblasts by a FAP-specific T-cell activator (FAP-TAc) in the presence or absence of combinations of IL-12, IL15 and IL-18.

FIG. 2B shows enhancement by IFNα of the of PBMC-derived T-cell mediated killing (apoptosis induction) of target cell fibroblasts by a FAP-specific T-cell activator (FAP-TAc) in the presence or absence of combinations of IL-12, IL15 and IL-18.

FIG. 2C shows kinetic analysis of T-cell mediated killing (apoptosis induction) of target cell fibroblasts by primary tumour-derived lymphocytes (kidney tumour 70) stimulated with a FAP-specific T-cell activator (FAP-TAc) in the presence or absence of combinations of IL-12, IL15 and IL-18.

FIG. 2D shows the enhancement by IFNα of the T-cell mediated killing (apoptosis induction) of target cell fibroblasts by primary tumour-derived lymphocytes (kidney tumour 70) stimulated with a FAP-specific T-cell activator (FAP-TAc) in the presence or absence of combinations of IL-12, IL15 and IL-18.

FIG. 2E shows the effect of IL-12 and IL-15 on PBMC-derived NK cell-mediated killing of K562 target tumour cells.

FIG. 2F shows the effect of IL-12, IL-15 and IFNα on primary tumour-derived NK cell-mediated killing of K562 target tumour cells.

FIG. 3A shows chemokine stimulated migration of naïve T-cells prepared from PBMCs in culture using recombinant chemokines.

FIG. 3B shows chemokine stimulated migration of effector T-cells prepared from PBMCs in culture using recombinant chemokines.

FIG. 3C shows chemokine stimulated migration of CD45+TILS, prepared from a primary breast tumour sample (53), in culture using recombinant chemokines.

FIG. 3D shows chemokine stimulated migration of CD3+ T-cells and non-T-cells (CD3-) from primary lymph nodes from breast cancer surgery.

FIG. 3E shows chemokine stimulated migration of monocyte-derived dendritic cells across a Matrigel coated transwell.

FIG. 3F shows real-time imaging analysis (Incucyte) of CCL19 and CCL21 chemokine stimulated migration of dendritic cells across a Matrigel coated transwell.

FIG. 4A shows effect of IL-15 with or without IL-12 on primary lymph node T-cell responses to Muc-1 tumour antigen or CEFT peptides measure by IFNg ELISPOT assay.

FIG. 4B shows the effect of IL-15 with or without IL-12 on primary lymph node T-cell responses to Her2 tumour antigens (HER2-ECD and HER2-ICD) or CEFT peptides measure by IFNg ELISPOT assay.

FIG. 5A shows a schematic representation of transgene cassettes encoding human IL-12 as separate p35 and p40 proteins or as single chain IL-12 molecules which use a linker to covalently join the p35 and p40 chains.

FIG. 5B shows the genome replication of NG-701, NG-702 and NG-703 in A549 cells.

FIG. 5C shows IL-12 p70 protein production by A549 cells infected with NG-701, NG-702 and NG-703

FIG. 5D shows RT-qPCR analysis of transgene mRNA expression by NG-701, NG-702 and NG-703 inoculated A549 cells.

FIG. 5E shows IL-12p40 and IL-12p70 production measured by ELISA assay of supernatants from A549 cells inoculated with NG-701 or NG-702.

FIG. 5F shows functional activity of IL-12 produced by NG-702 or NG-703 inoculated A549 cells assessed with a HEK-Blue cell IL-12 signaling reporter assay.

FIG. 5G shows the effect of recombinant human IL-12 or supernatants from NG-702 infected A549 cells on CD107a expression by CD4+ T-cells stimulated with anti-CD3 and/or anti-CD28 antibodies.

FIG. 5H shows the effect of recombinant human IL-12 or supernatants from NG-702 infected A549 cells on CD107a expression by CD8+ T-cells stimulated with anti-CD3 and/or anti-CD28 antibodies.

FIG. 6 shows a schematic representation of transgene cassettes encoding the IL12p40-Linker-IL12p35 single chain human IL-12 plus one or more other transgenes. In one embodiment these constructs are inserted in position B_{Y}.

FIG. 7A shows genome replication of EnAd, NG-702, NG-704 and NG-706 in A549 cells.

FIG. 7B shows IL-12p70 protein levels produced by A549 cells inoculated with NG-707, NG-704 or NG-706.

FIG. 7C shows IFNa protein levels produced by the same A549 cells treated with NG-704 or NG-706.

FIG. 7D shows the expression of mRNA for Flt3L, MIP1a, IFNα, CXCL9 and IL-12 transgenes in A549 cells inoculated with NG-707.

FIG. 7E shows the expression of Flt3L, MIP1a, IFNα, CXCL9 and IL-12 transgene proteins by A549 cells inoculated with NG-707.

FIG. 7F shows the expression of IFNα, CCL19, IL-18 and IL-12 transgene proteins by A549 cells inoculated with NG-709

FIG. 7G shows functional IL-12 activity in supernatants of A549 cells inoculated with NG-704, NG-706, NG-707 or NG-709.

FIG. 7H shows production of IL-12, Flt3L and CCL21 transgene proteins by A549 cells inoculated with NG-708.

FIG. 7I shows encoded transgene protein production by NG-708 and NG-709 inoculated A549 cells measured by specific ELISAs.

FIG. 7J shows encoded transgene protein production by A549 cells inoculated with different viruses depicted in FIG. 7A above (1×10⁶ cells).

FIG. 8A shows production of IL-12 p70 protein by tumour cell preparations from a primary colorectal (68) and a kidney (70) tumour inoculated with NG-702 or NG-704.

FIG. 8B shows production of IFNg by a primary kidney tumour cell preparation cultured with different combinations of IL-12, IL-15 and IL-18, with or without inoculation with EnAd, NG-702 or NG-704 viruses.

FIG. 8C shows time course of IL-12 p70 protein production by tumour cell preparations from a kidney (70) and a colorectal (71) primary tumour cell preparations inoculated with NG-707.

FIG. 8D shows production of IL-12 p70 protein by primary breast tumour cell preparations (66 and 67) inoculated with NG-702, NG-704, NG-707 or NG-709.

FIG. 8E shows time course of IL-12 p70 protein production by a primary colorectal tumour (75) cell preparation inoculated with NG-707 at two different dose levels.

FIG. 8F shows time course of IL-12 p70 protein production by a primary colorectal tumour (76) cell preparation inoculated with NG-707 at two different dose levels.

FIG. 8G shows time course of IL-12 p70 protein production by a primary colorectal tumour (79) cell preparation inoculated with NG-707 at two different dose levels.

FIG. 8H shows time course of IL-12 p70 protein production by a primary kidney tumour (tumour 70) cell preparation inoculated with NG-707 at two different dose levels.

FIG. 9A shows schematic representation of transgene cassettes encoding transmembrane or soluble secreted forms of IL-15 receptor alpha sushi domain with or without IL-15.

FIG. 9B shows production of IL-15 by NG-740 compared to three viruses not expressing an IL-15 receptor alpha form.

FIG. 9C shows IL-15 production following co transfection of an IL-15 plasmid with either transmembrane form of IL-15 receptor alpha sushi domain (sushi-TM) or a soluble secreted version (sushi).

FIG. 9D shows functional activity of IL-15 in samples from FIG. 9C.

FIG. 9E shows IL-15 production by A549 cells inoculated with NG-740 and NG-748 expressing transmembrane or soluble secreted forms of IL-15 receptor alpha sushi domain, respectively

FIG. 9F shows IL-15 production by primary colorectal tumour cells inoculated with NG-740 and NG-748 expressing transmembrane or soluble secreted forms of IL-15 receptor alpha sushi domain, respectively.

FIG. 9G shows IFNg production by primary kidney tumour cells treated with NG-748 or NG-702, in presence of different IL-12 or IL-15 respectively.

FIG. 10A shows schematic representation of transgene cassettes encoding IL-12 and IL-15 together with transmembrane or soluble secreted forms of IL-15 receptor alpha sushi domain.

FIG. 10B shows IL-12 and IL-15 production by A549 cells treated with the viruses depicted in FIG. 10A, measured by ELISA.

FIG. 10C shows functional activity of IL-12 and IL-15 from same samples as FIG. 10B.

FIG. 10D shows IL-12, IL-15 and IFNg production by A549 cells inoculated with different viruses, with PBMCs added to the cultures after 24h.

FIG. 10E shows IL-12, IL-15 and IFNg production by A549 cells inoculated with different viruses, with purified CD3+T_cells added to the cultures after 24h.

FIG. 10F shows IFNg production by T-cells cultured in direct contact with, or separated in a transwell format, from A549 cells treated with different viruses.

FIG. 10G shows IL-15 and IFNg production by A549 cells inoculated with different viruses, with PBMCs or purified T-cells added to the cultures after 24h.

FIG. 11A shows production of IFNg, IL-12 and IL-15 by primary colorectal tumour cells (tumour 98) 72h following treatment with different viruses.

FIG. 11B shows production of IFNg by primary colorectal tumour cells (tumour 99) 96h following treatment with different viruses.

FIG. 12A shows a schematic representation of transgene cassettes encoding IL-12 and IL-15 together with transmembrane or soluble secreted forms of IL-15 receptor alpha sushi domain and a further chemokine transgene (CXCL9 or CCL21).

FIG. 12B shows the production of IL-12p70, IL-15, CXCL9 and CCL21 by A549 cells treated with different viruses.

FIG. 12C shows the production of IL-15 by A549 cells treated with different viruses.

FIG. 12D shows the production of IL-12p70, IL-15 and CXCL9 transgene proteins and IFNg cytokine secretion by primary tumour cells (tumour 105) treated with different viruses.

FIG. 12E shows the production of IL-12p70, IL-15, CXCL9 and IFNg by primary colorectal tumour cells (tumour 105) treated with different viruses.

FIG. 12F shows the production of IL-12p70, IL-15, CXCL9 CCL21 and IFNg by primary colorectal tumour cells (tumour 107) treated with different viruses.

FIG. 12G shows the migration of monocyte-derived dendritic cells stimulated by CCL21 transgene protein in supernatants from A549 cells treated with NG-795A and migration inhibition in the presence of added anti-CCL21 antibody.

FIG. 12H shows the migration of monocyte-derived dendritic cells stimulated by CCL21 transgene protein in supernatants from A549 cells treated with NG-795A and selective migration inhibition in the presence of added anti-CCL21 antibody.

FIG. 13 shows the levels of IL-12 p70 in the plasma of human tumour xenograft-bearing mice injected with NG-786A, NG-791A or NG-796A compared with those dosed with EnAd or untreated mice.

FIG. 14 shows the generic structure of chimeric antigen receptors.

FIG. 15 shows a graph of the migration of monocyte-derived dendritic cells stimulated by CCL21 transgene protein in supernatants from A549 cells treated with NG-796A and migration inhibition in the presence of added anti-CCL21 antibody.

FIG. 16A shows the control of A549 tumour xenograft growth by IV dosed NG-704 prior to transfer of Her2-specific CAR-T cells compared to CAR-T alone of CAR-T plus EnAd pre-dosing.

FIG. 16B shows the control of A549 tumour xenograft growth by IV dosed NG-796A prior to transfer of HER2-specific CAR-T cells compared to CAR-T alone of CAR-T plus EnAd pre-dosing.

FIG. 16C shows the levels of CCL21 in A549 xenograft tumours following IV dosing with NG-796A or EnAd.

FIG. 17 shows the levels of IL-12, CCL21, IL-15 and IL-15Ra sushi domain protein in supernatants of A549 cells infected with NG-796A.

FIG. 18A shows the levels of IL-15 detected in supernatants of A549 cells transfected with pUC-796A or pRES-128 with or without different concentrations of recombinant IL-15Ra sushi domain protein added to the cultures.

FIG. 18B shows a schematic representation of transgene cassettes in CMV pUC vectors encoding single chain IL-12 (scIL12), CCL21 and IL-15 with (pUC-796A) or without (pRES-128) a sequence encoding secreted IL-15Ra sushi domain

### SEQUENCES

SEQ ID NO: 1 Short splice acceptor (SSA) DNA sequence -CAGG
SEQ ID NO: 2 Splice acceptor (SA) DNA sequence
SEQ ID NO: 3 Splice acceptor DNA sequence
SEQ ID NO: 4 High efficiency self-cleavable P2A peptide sequence
SEQ ID NO: 5 High efficiency self-cleavable T2A peptide sequence
SEQ ID NO: 6 High efficiency self-cleavable E2A peptide sequence
SEQ ID NO: 7 High efficiency self-cleavable F2A peptide sequence
SEQ ID NO: 8 Poly adenylation (PA) sequence (SV40 late polyA sequence)
SEQ ID NO: 9 Human IL-12 p35 protein with signal sequence (IL12p35)
SEQ ID NO: 10 Human IL-12 p40 protein with signal sequence (IL12p40)
SEQ ID NO: 11 Human IL-12 p70 single chain protein with a (Gly₄Ser)₃ linker joining the IL12p40 protein with its signal sequence N-terminal to the IL12p35 protein without its signal sequence (IL12p40LinkerIL12p35)
SEQ ID NO: 12 Human IL-12 p70 single chain protein with a (Gly₄Ser)₃ linker joining the IL12p35 protein with its signal sequence N-terminal to the IL12p40 protein without its signal sequence (IL12p35LinkerIL12p40)
SEQ ID NO: 13 Human Interferon alpha 2 protein sequence
SEQ ID NO: 14 Human Fms-related tyrosine kinase 3 ligand (Flt3L) soluble extracellular domain protein sequence
SEQ ID NO: 15 Human MIP1alpha LD78beta (MIP1a) protein sequence
SEQ ID NO: 16 Human CXCL9 protein sequence
SEQ ID NO: 17 Human CCL21 protein sequence
SEQ ID NO: 18 Human CCL5 protein sequence
SEQ ID NO: 19 Human CCL19 protein sequence
SEQ ID NO: 20 Human IL-18 long isoform
SEQ ID NO: 21 Human IL-15 protein with N-terminal human Ig leader sequence
SEQ ID NO: 22 Human Ig leader peptide sequence
SEQ ID NO: 23 Human IL-15 protein sequence without leader sequence
SEQ ID NO: 24 Human CCL21 C-terminally truncated protein sequence (CCL21t)
SEQ ID NO: 25 Human IL-15 receptor alpha Sushi domain protein sequence, with IL-15 receptor alpha leader sequence at N-terminus, with a C-terminal myc peptide linking it to a PDGF receptor transmembrane domain (IL15RsushimycTM)
SEQ ID NO: 26 Human IL-15 receptor alpha Sushi domain protein sequence with IL-15 receptor alpha leader sequence at N-terminus
SEQ ID NO: 27 Myc peptide sequence
SEQ ID NO: 28 PDGF receptor transmembrane domain protein sequence
SEQ ID NO: 29 Human IL-15 receptor alpha Sushi domain protein sequence, with IL-15 receptor alpha leader sequence at N-terminus, with a Gly₄Ser C-terminal peptide sequence linking it to a PDGF receptor transmembrane domain (IL15RsushiG4STM)
SEQ ID NO: 30 Gly₄Ser peptide linker sequence
SEQ ID NO: 31 (Gly₄Ser)₃ peptide linker sequence
SEQ ID NO: 32 Human CCL21 coding DNA sequence encoding SEQ ID NO: 17
SEQ ID NO: 33 Human CCL21 modified codon sequence (CCL21mod) encoding same protein sequence as encoded by SEQ ID NO: 32
SEQ ID NO: 34 Human CCL21 coding sequence truncated at the 3′ end to produce a C-terminally truncated protein sequence (SEQ ID NO: 24)
SEQ ID NO: 35 Human CCL21 modified coding sequence truncated at the 3′ end (CCL21tmod) to produce a C-terminally truncated protein sequence (SEQ ID NO: 24), the same protein sequence as encoded by SEQ ID NO: 34
SEQ ID NO: 36 Protein sequence encoded by NG-704 transgene cassette
SEQ ID NO: 37 Protein sequence encoded by NG-706 transgene cassette
SEQ ID NO: 38 Protein sequence encoded by NG-707 transgene cassette
SEQ ID NO: 39 Protein sequence encoded by NG-708 transgene cassette
SEQ ID NO: 40 Protein sequence encoded by NG-709 transgene cassette
SEQ ID NO: 41 Protein sequence encoded by NG-720 transgene cassette
SEQ ID NO: 42 Protein sequence encoded by NG-721 transgene cassette
SEQ ID NO: 43 Protein sequence encoded by NG-722 transgene cassette
SEQ ID NO: 44 Protein sequence encoded by NG-723 transgene cassette
SEQ ID NO: 45 Protein sequence encoded by NG-724 transgene cassette
SEQ ID NO: 46 Protein sequence encoded by NG-725 transgene cassette
SEQ ID NO: 47 Protein sequence encoded by NG-726 transgene cassette
SEQ ID NO: 48 Protein sequence encoded by NG-740 transgene cassette
SEQ ID NO: 49 Protein sequence encoded by NG-742 transgene cassette
SEQ ID NO: 50 Protein sequence encoded by NG-744 transgene cassette
SEQ ID NO: 51 Protein sequence encoded by NG-746 transgene cassette
SEQ ID NO: 52 Protein sequence encoded by NG-750 transgene cassette
SEQ ID NO: 53 Protein sequence encoded by NG-751 transgene cassette
SEQ ID NO: 54 Protein sequence encoded by NG-752 transgene cassette
SEQ ID NO: 55 Protein sequence encoded by NG-753 transgene cassette
SEQ ID NO: 56 Protein sequence encoded by NG-754 transgene cassette
SEQ ID NO: 57 Protein sequence encoded by NG-755 transgene cassette
SEQ ID NO: 58 Protein sequence encoded by NG-756 transgene cassette
SEQ ID NO: 59 Protein sequence encoded by NG-757 transgene cassette
SEQ ID NO: 60 Protein sequence encoded by NG-758 transgene cassette
SEQ ID NO: 61 Protein sequence encoded by NG-759 transgene cassette
SEQ ID NO: 62 Protein sequence encoded by NG-760 transgene cassette
SEQ ID NO: 63 Protein sequence encoded by NG-761 transgene cassette
SEQ ID NO: 64 Protein sequence encoded by NG-762 transgene cassette
SEQ ID NO: 65 Protein sequence encoded by NG-763 transgene cassette
SEQ ID NO: 66 Protein sequence encoded by NG-764 transgene cassette
SEQ ID NO: 67 Protein sequence encoded by NG-765 transgene cassette
SEQ ID NO: 68 Protein sequence encoded by NG-768 transgene cassette
SEQ ID NO: 69 Protein sequence encoded by NG-769 transgene cassette
SEQ ID NO: 70 Protein sequence encoded by NG-770 transgene cassette
SEQ ID NO: 71 Protein sequence encoded by NG-771 transgene cassette
SEQ ID NO: 72 Protein sequence encoded by NG-772 transgene cassette
SEQ ID NO: 73 Protein sequence encoded by NG-773 transgene cassette
SEQ ID NO: 74 Genome sequence of NG-701 virus
SEQ ID NO: 75 Genome sequence of NG-702 virus
SEQ ID NO: 76 Genome sequence of NG-703 virus
SEQ ID NO: 77 Genome sequence of NG-704 virus
SEQ ID NO: 78 Genome sequence of NG-706 virus
SEQ ID NO: 79 Genome sequence of NG-707 virus
SEQ ID NO: 80 Genome sequence of NG-708 virus
SEQ ID NO: 81 Genome sequence of NG-709 virus
SEQ ID NO: 82 Genome sequence of NG-720 virus
SEQ ID NO: 83 Genome sequence of NG-721 virus
SEQ ID NO: 84 Genome sequence of NG-722 virus
SEQ ID NO: 85 Genome sequence of NG-723 virus
SEQ ID NO: 86 Genome sequence of NG-724 virus
SEQ ID NO: 87 Genome sequence of NG-725 virus
SEQ ID NO: 88 Genome sequence of NG-726 virus
SEQ ID NO: 89 Genome sequence of NG-740 virus
SEQ ID NO: 90 Genome sequence of NG-742 virus
SEQ ID NO: 91 Genome sequence of NG-744 virus
SEQ ID NO: 92 Genome sequence of NG-746 virus
SEQ ID NO: 93 Genome sequence of NG-750 virus
SEQ ID NO: 94 Genome sequence of NG-751 virus
SEQ ID NO: 95 Genome sequence of NG-752 virus
SEQ ID NO: 96 Genome sequence of NG-753 virus
SEQ ID NO: 97 Genome sequence of NG-754 virus
SEQ ID NO: 98 Genome sequence of NG-755 virus
SEQ ID NO: 99 Genome sequence of NG-756 virus
SEQ ID NO: 100 Genome sequence of NG-757 virus
SEQ ID NO: 101 Genome sequence of NG-758 virus
SEQ ID NO: 102 Genome sequence of NG-759 virus
SEQ ID NO: 103 Genome sequence of NG-760 virus
SEQ ID NO: 104 Genome sequence of NG-761 virus
SEQ ID NO: 105 Genome sequence of NG-762 virus
SEQ ID NO: 106 Genome sequence of NG-763 virus
SEQ ID NO: 107 Genome sequence of NG-764 virus
SEQ ID NO: 108 Genome sequence of NG-765 virus
SEQ ID NO: 109 Genome sequence of NG-768 virus
SEQ ID NO: 110 Genome sequence of NG-769 virus
SEQ ID NO: 111 Genome sequence of NG-770 virus
SEQ ID NO: 112 Genome sequence of NG-771 virus
SEQ ID NO: 113 Genome sequence of NG-772 virus
SEQ ID NO: 114 Genome sequence of NG-773 virus
SEQ ID NO: 115 Mature human IL-12 p70 single chain protein with a (Gly₄Ser)₃ linker joining the IL12p40 protein without its signal sequence N-terminal to the IL12p35 protein without its signal sequence (mature IL12p40LinkerIL12p35)
SEQ ID NO: 116 DNA sequence for the protein coding sequence of the NG-701 transgene cassette, including the stop codon
SEQ ID NO: 117 DNA sequence for the protein coding sequence of the NG-702 transgene cassette, including the stop codon
SEQ ID NO: 118 DNA sequence for the protein coding sequence of the NG-703 transgene cassette, including the stop codon
SEQ ID NO: 119 DNA sequence for the protein coding sequence of the NG-704 transgene cassette, including the stop codon
SEQ ID NO: 120 DNA sequence for the protein coding sequence of the NG-706 transgene cassette, including the stop codon
SEQ ID NO: 121 DNA sequence for the protein coding sequence of the NG-707 transgene cassette, including the stop codon
SEQ ID NO: 122 DNA sequence for the protein coding sequence of the NG-708 transgene cassette, including the stop codon
SEQ ID NO: 123 DNA sequence for the protein coding sequence of the NG-709 transgene cassette, including the stop codon
SEQ ID NO: 124 DNA sequence for the protein coding sequence of the NG-720 transgene cassette, including the stop codon
SEQ ID NO: 125 DNA sequence for the protein coding sequence of the NG-721 transgene cassette, including the stop codon
SEQ ID NO: 126 DNA sequence for the protein coding sequence of the NG-722 transgene cassette, including the stop codon
SEQ ID NO: 127 DNA sequence for the protein coding sequence of the NG-723 transgene cassette, including the stop codon
SEQ ID NO: 128 DNA sequence for the protein coding sequence of the NG-724 transgene cassette, including the stop codon
SEQ ID NO: 129 DNA sequence for the protein coding sequence of the NG-725 transgene cassette, including the stop codon
SEQ ID NO: 130 DNA sequence for the protein coding sequence of the NG-726 transgene cassette, including the stop codon
SEQ ID NO: 131 DNA sequence for the protein coding sequence of the NG-740 transgene cassette, including the stop codon
SEQ ID NO: 132 DNA sequence for the protein coding sequence of the NG-742 transgene cassette, including the stop codon
SEQ ID NO: 133 DNA sequence for the protein coding sequence of the NG-744 transgene cassette, including the stop codon
SEQ ID NO: 134 DNA sequence for the protein coding sequence of the NG-746 transgene cassette, including the stop codon
SEQ ID NO: 135 DNA sequence for the protein coding sequence of the NG-750 transgene cassette, including the stop codon
SEQ ID NO: 136 DNA sequence for the protein coding sequence of the NG-751 transgene cassette, including the stop codon
SEQ ID NO: 137 DNA sequence for the protein coding sequence of the NG-752 transgene cassette, including the stop codon
SEQ ID NO: 138 DNA sequence for the protein coding sequence of the NG-753 transgene cassette, including the stop codon
SEQ ID NO: 139 DNA sequence for the protein coding sequence of the NG-754 transgene cassette, including the stop codon
SEQ ID NO: 140 DNA sequence for the protein coding sequence of the NG-755 transgene cassette, including the stop codon
SEQ ID NO: 141 DNA sequence for the protein coding sequence of the NG-756 transgene cassette, including the stop codon
SEQ ID NO: 142 DNA sequence for the protein coding sequence of the NG-757 transgene cassette, including the stop codon
SEQ ID NO: 143 DNA sequence for the protein coding sequence of the NG-758 transgene cassette, including the stop codon
SEQ ID NO: 144 DNA sequence for the protein coding sequence of the NG-759 transgene cassette, including the stop codon
SEQ ID NO: 145 DNA sequence for the protein coding sequence of the NG-760 transgene cassette, including the stop codon
SEQ ID NO: 146 DNA sequence for the protein coding sequence of the NG-761 transgene cassette, including the stop codon
SEQ ID NO: 147 DNA sequence for the protein coding sequence of the NG-762 transgene cassette, including the stop codon
SEQ ID NO: 148 DNA sequence for the protein coding sequence of the NG-763 transgene cassette, including the stop codon
SEQ ID NO: 149 DNA sequence for the protein coding sequence of the NG-764 transgene cassette, including the stop codon
SEQ ID NO: 150 DNA sequence for the protein coding sequence of the NG-765 transgene cassette, including the stop codon
SEQ ID NO: 151 DNA sequence for the protein coding sequence of the NG-768 transgene cassette, including the stop codon
SEQ ID NO: 152 DNA sequence for the protein coding sequence of the NG-769 transgene cassette, including the stop codon
SEQ ID NO: 153 DNA sequence for the protein coding sequence of the NG-770 transgene cassette, including the stop codon
SEQ ID NO: 154 DNA sequence for the protein coding sequence of the NG-771 transgene cassette, including the stop codon
SEQ ID NO: 155 DNA sequence for the protein coding sequence of the NG-772 transgene cassette, including the stop codon
SEQ ID NO: 156 DNA sequence for the protein coding sequence of the NG-773 transgene cassette, including the stop codon
SEQ ID NO: 157 PDGF receptor A transmembrane domain amino acid sequence
SEQ ID NO: 158 PDGF receptor B transmembrane domain amino acid sequence
SEQ ID NO: 159 Insulin-like growth factor 1 receptor transmembrane domain amino acid sequence
SEQ ID NO: 160 IL-6 receptor transmembrane domain amino acid sequence
SEQ ID NO: 161 CD28 transmembrane domain amino acid sequence
SEQ ID NO: 162 Sequence comprising a start codon—(gcc)gccRccAUGg
SEQ ID NO: 163 Human IL-15 protein with N-terminal human CD33 leader sequence
SEQ ID NO: 164 Human CD33 leader peptide sequence
SEQ ID NO: 165 Human IL-15 protein with N-terminal human IL-2 leader sequence
SEQ ID NO: 166 Human IL-2 leader peptide sequence
SEQ ID NO: 167 Protein sequence encoded by NG-748 transgene cassette
SEQ ID NO: 168 Protein sequence encoded by NG-774 transgene cassette
SEQ ID NO: 169 Protein sequence encoded by NG-775 transgene cassette
SEQ ID NO: 170 Protein sequence encoded by NG-776 transgene cassette
SEQ ID NO: 171 Protein sequence encoded by NG-777 transgene cassette
SEQ ID NO: 172 Protein sequence encoded by NG-781 transgene cassette
SEQ ID NO: 173 Protein sequence encoded by NG-782 transgene cassette
SEQ ID NO: 174 Protein sequence encoded by NG-784 transgene cassette
SEQ ID NO: 175 Protein sequence encoded by NG-785 transgene cassette
SEQ ID NO: 176 Protein sequence encoded by NG-785A transgene cassette
SEQ ID NO: 177 Protein sequence encoded by NG-786A transgene cassette
SEQ ID NO: 178 Protein sequence encoded by NG-787 transgene cassette
SEQ ID NO: 179 Protein sequence encoded by NG-787A transgene cassette
SEQ ID NO: 180 Protein sequence encoded by NG-788P transgene cassette
SEQ ID NO: 181 Protein sequence encoded by NG-789P transgene cassette
SEQ ID NO: 182 Protein sequence encoded by NG-790P transgene cassette
SEQ ID NO: 183 Protein sequence encoded by NG-791A transgene cassette
SEQ ID NO: 184 Protein sequence encoded by NG-792A transgene cassette
SEQ ID NO: 185 Protein sequence encoded by NG-794A transgene cassette
SEQ ID NO: 186 Protein sequence encoded by NG-795A transgene cassette
SEQ ID NO: 187 Protein sequence encoded by NG-796A transgene cassette
SEQ ID NO: 188 Protein sequence encoded by NG-799A transgene cassette
SEQ ID NO: 189 DNA sequence for the protein coding sequence of the NG-748 transgene cassette, including the stop codon
SEQ ID NO: 190 DNA sequence for the protein coding sequence of the NG-774 transgene cassette, including the stop codon
SEQ ID NO: 191 DNA sequence for the protein coding sequence of the NG-775 transgene cassette, including the stop codon
SEQ ID NO: 192 DNA sequence for the protein coding sequence of the NG-776 transgene cassette, including the stop codon
SEQ ID NO: 193 DNA sequence for the protein coding sequence of the NG-777 transgene cassette, including the stop codon
SEQ ID NO: 194 DNA sequence for the protein coding sequence of the NG-781 transgene cassette, including the stop codon
SEQ ID NO: 195 DNA sequence for the protein coding sequence of the NG-782 transgene cassette, including the stop codon
SEQ ID NO: 196 DNA sequence for the protein coding sequence of the NG-784 transgene cassette, including the stop codon
SEQ ID NO: 197 DNA sequence for the protein coding sequence of the NG-785 transgene cassette, including the stop codon
SEQ ID NO: 198 DNA sequence for the protein coding sequence of the NG-785A transgene cassette, including the stop codon
SEQ ID NO: 199 DNA sequence for the protein coding sequence of the NG-786A transgene cassette, including the stop codon
SEQ ID NO: 200 DNA sequence for the protein coding sequence of the NG-787 transgene cassette, including the stop codon
SEQ ID NO: 201 DNA sequence for the protein coding sequence of the NG-787A transgene cassette, including the stop codon
SEQ ID NO: 202 DNA sequence for the protein coding sequence of the NG-788P transgene cassette, including the stop codon
SEQ ID NO: 203 DNA sequence for the protein coding sequence of the NG-789P transgene cassette, including the stop codon
SEQ ID NO: 204 DNA sequence for the protein coding sequence of the NG-790P transgene cassette, including the stop codon
SEQ ID NO: 205 DNA sequence for the protein coding sequence of the NG-791A transgene cassette, including the stop codon
SEQ ID NO: 206 DNA sequence for the protein coding sequence of the NG-792A transgene cassette, including the stop codon
SEQ ID NO: 207 DNA sequence for the protein coding sequence of the NG-794A transgene cassette, including the stop codon
SEQ ID NO: 208 DNA sequence for the protein coding sequence of the NG-795A transgene cassette, including the stop codon
SEQ ID NO: 209 DNA sequence for the protein coding sequence of the NG-796A transgene cassette, including the stop codon
SEQ ID NO: 210 DNA sequence for the protein coding sequence of the NG-799A transgene cassette, including the stop codon
SEQ ID NO: 211 Genome sequence of NG-748 virus
SEQ ID NO: 212 Genome sequence of NG-774 virus
SEQ ID NO: 213 Genome sequence of NG-775 virus
SEQ ID NO: 214 Genome sequence of NG-776 virus
SEQ ID NO: 215 Genome sequence of NG-777 virus
SEQ ID NO: 216 Genome sequence of NG-781 virus
SEQ ID NO: 217 Genome sequence of NG-782 virus
SEQ ID NO: 218 Genome sequence of NG-784 virus
SEQ ID NO: 219 Genome sequence of NG-785 virus
SEQ ID NO: 220 Genome sequence of NG-785A virus
SEQ ID NO: 221 Genome sequence of NG-786A virus
SEQ ID NO: 222 Genome sequence of NG-787 virus
SEQ ID NO: 223 Genome sequence of NG-787A virus
SEQ ID NO: 224 Genome sequence of NG-788P virus
SEQ ID NO: 225 Genome sequence of NG-789P virus
SEQ ID NO: 226 Genome sequence of NG-790P virus
SEQ ID NO: 227 Genome sequence of NG-791A virus
SEQ ID NO: 228 Genome sequence of NG-792A virus
SEQ ID NO: 229 Genome sequence of NG-794A virus
SEQ ID NO: 230 Genome sequence of NG-795A virus
SEQ ID NO: 231 Genome sequence of NG-796A virus
SEQ ID NO: 232 Genome sequence of NG-799A virus
SEQ ID NO: 233 Protein sequence encoded by NG-701 transgene cassette
SEQ ID NO: 234 Protein sequence encoded by NG-702 transgene cassette
SEQ ID NO: 235 Protein sequence encoded by NG-703 transgene cassette
SEQ ID NO: 236 Human IL-15 protein with N-terminal human Ig leader sequence with a peptide linker joining it to the Human IL-15 receptor alpha Sushi domain without its' signal sequence
SEQ ID NO: 237 Linker peptide joining IL-15 to IL-15 receptor alpha sushi domain in NG-787 and NG-787A
SEQ ID NO: 238 Human PDGFR Receptor A transmembrane region peptide
SEQ ID NO: 239 Human PDGFR Receptor B transmembrane region peptide
SEQ ID NO: 240 Human Insulin-Like Growth Factor 1 transmembrane region peptide
SEQ ID NO: 241 Human IL6-R transmembrane region peptide
SEQ ID NO: 242 Human CD28 transmembrane region peptide
SEQ ID NO: 243 Human IL-15 receptor alpha Sushi domain protein sequence
SEQ ID NO: 244 Protein sequence of recombinant N-terminally His-tagged IL-15Ra sushi domain protein with C-terminal P2A peptide sequence
SEQ ID NO: 245 DNA sequence of CMV pUC vector pUC-796A
SEQ ID NO: 246 DNA sequence of CMV pUC vector pRES-128

### EXAMPLES

### EXAMPLE 1: Activity of Recombinant IL-15, IL-18 and IFNα Proteins in Primary Human Tumour Samples

To evaluate potential additive or synergistic activities of different combinations of cytokines and/or chemokines on primary tumour cell culture responses, recombinant proteins were used to model molecules encoded as transgenes in viruses. For effects on primary human tumour cells, surgically excised tumour samples were placed in Aqix organ transportation medium (supplemented with amphotericin B, penicillin, streptomycin, gentamycin and metronidazole) and shipped from the clinical site at 4° C. and obtained for processing in the laboratory within 24h. Samples were cut into small pieces using scalpels and then enzymatically dissociated using a tumour dissociation mix (Miltenyi Biotech) on a Gentle MACS tissue disruptor (Miltenyi Biotech). Single cell suspensions were obtained by filtering and plated into 96 well or 24 well plates, depending on the cell yield in either RPMI (Gibco) supplemented with foetal bovine serum, L-glutamine, sodium pyruvate and non-essential amino acids, or Cancer Cell Line Medium XF (PromoCell). Both media formulations were additionally supplemented with amphotericin B, penicillin and streptomycin. Cell types contained in these suspensions were routinely characterized by flow cytometry and shown to include tumour cells and different immune cell subsets, including T cells, B cells and NK cells.

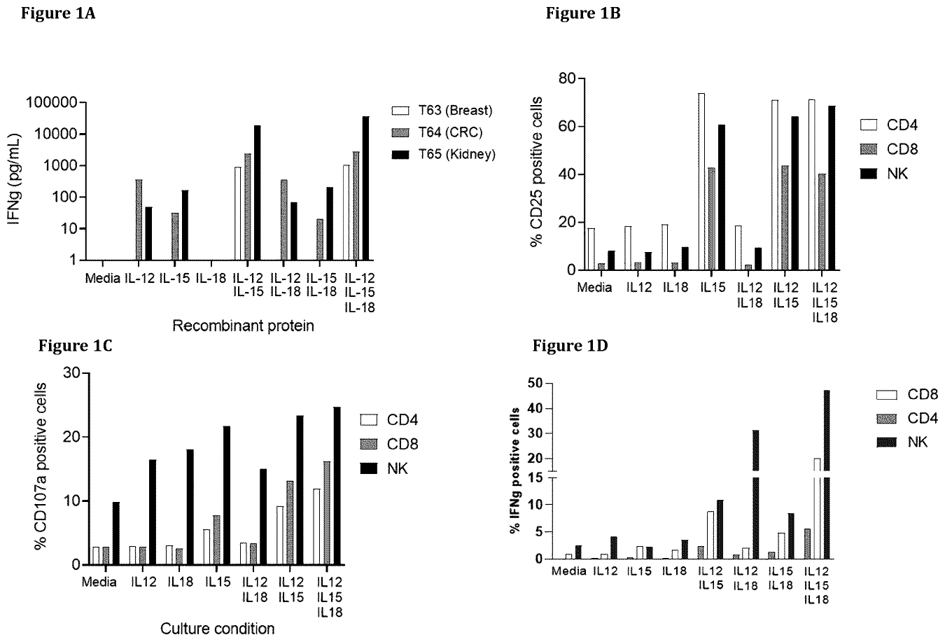

Three primary tumour samples, tumour 63 (breast), tumour 64 (CRC) and tumour 65 (kidney) were received and processed as described above. Recombinant IL-12 p70 (15 ng/mL, R&D Systems), IL-18 (50 ng/mL, RnD Systems) and IL-15 (50 ng/mL, InvivoGen) were added either alone or in combination to the dissociated tumour cell cultures. Supernatant samples were harvested 72 hours after stimulation and were clarified as described previously. IFNγ protein was quantified by ELISA. Both IL-12 and IL-15 individually stimulated IFNγ production from tumour 64 and 65 cultures (but not 63), but the combination of IL-12+IL-15, or IL-12+IL-15+IL-18 stimulated the production of higher IFNγ levels than these stimuli alone, including from tumour 63 where no other combination produced detectable levels (FIG. 1A). In these tumour cell cultures, recombinant IL-18 did not stimulate IFNγ production and had little or no effect on the production of IFNγ triggered by IL-15 or the combination of IL-12 and IL-15.

In a second experiment, a breast tumour sample (tumour 60) was dissociated and cultured as described above with recombinant proteins. Live cells were harvested 72h post infection by scraping the monolayer and pipetting gently. Cells were pelleted by centrifugation at 300×g before adding 200 μL PBS containing 1 μL of Live-Dead Fixable Aqua (Life Technologies) and incubating on ice in the dark for 10 minutes. Samples were then pelleted by centrifugation before adding a cocktail of antibodies targeting several cell surface proteins (CD45, CD3, CD4, CD8, CD56, CD107a and CD25) in 50 μL cold PBS containing 2% FBS (flow buffer). Samples were incubated on ice in the dark for 20-30 minutes before being pelleted by centrifugation, washed twice in flow buffer and resuspended in flow buffer. Samples were then analysed by flow cytometry using an Attune NxT flow cytometer. Data showed that IL-15 alone, or in combination with IL-12 and IL-18, increased expression of the CD25 activation marker on CD4 and CD8 T cells, and NK cells (FIG. 1B). CD107a expression on NK cells was increased by the presence of IL-12, IL-15, IL-18 or combinations thereof. CD107a expression on CD4 and CD8 T cells was increased by the presence of IL-15, which was further enhanced by IL-12 or IL-12 plus IL-18. baseline (FIG. 1C).

In a third experiment, PBMCs from a healthy donor were cultured in RPMI (Gibco) supplemented with foetal bovine serum, L-glutamine, Na-Pyruvate and non-essential amino acids with the indicated recombinant proteins. Live cells were harvested 48 hours post stimulation and analysed by flow cytometry. However, in this case, 12 hours before harvesting, cells were treated with brefeldin A and an additional step of fixation/permeabilization was performed after the described above extracellular staining (using BD Cytofix/Cytoperm™ kit), after which cells were incubated with anti-IFNγ antibodies on ice in the dark for 30 minutes. Then cells were pelleted by centrifugation, washed twice in flow buffer and resuspended in flow buffer. Samples were analysed by flow cytometry using an Attune NxT flow cytometer. Data showed that IL-12 in combination with IL-15 increased the production of the IFNγ by CD4 and CD8 T cells and by NK cells, and this was further enhanced by the presence of IL-18 (FIG. 1D).

### EXAMPLE 2: Enhancement of T-Cell Mediated Killing of Target Cells by IL-12 with and without Other Recombinant Cytokines

To evaluate the potential for IL-12, IL-15, IL-18 and IFNα (alone or in combination) to enhance the T-cell mediated killing of target cell, we used a virus encoding a bispecific T cell activator (TAc), targeting both fibroblast activation protein (FAP) present on the surface of tumour-associated fibroblasts, and CD3 on T-cells (FAP-TAc) to drive T-cell mediated killing of FAP-expressing cells (Freedman et al, 2018 An Oncolytic Virus Expressing a T-cell Engager Simultaneously Targets Cancer and Immunosuppressive Stromal Cells. Cancer Res Nov 18:1-14; WO2018/041838 and WO2018/041827).

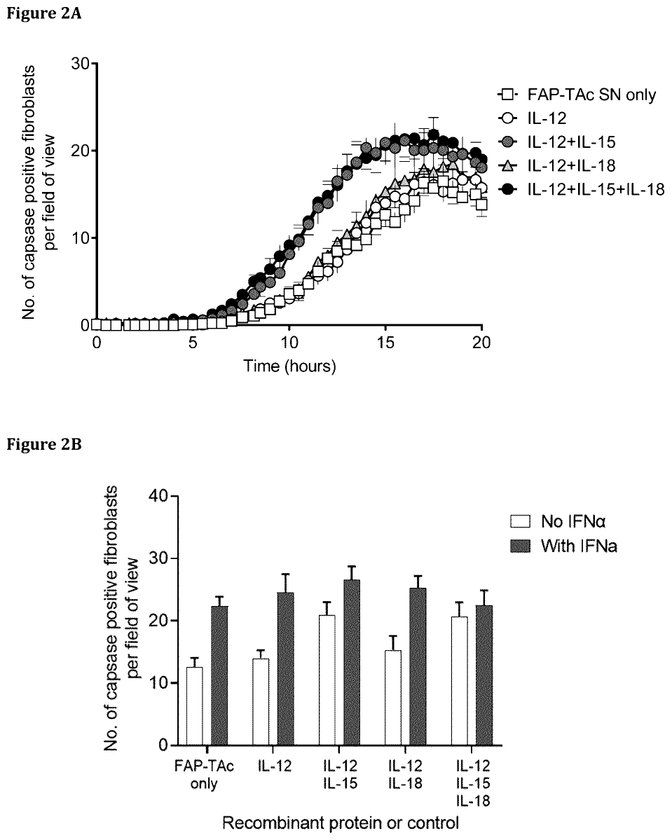

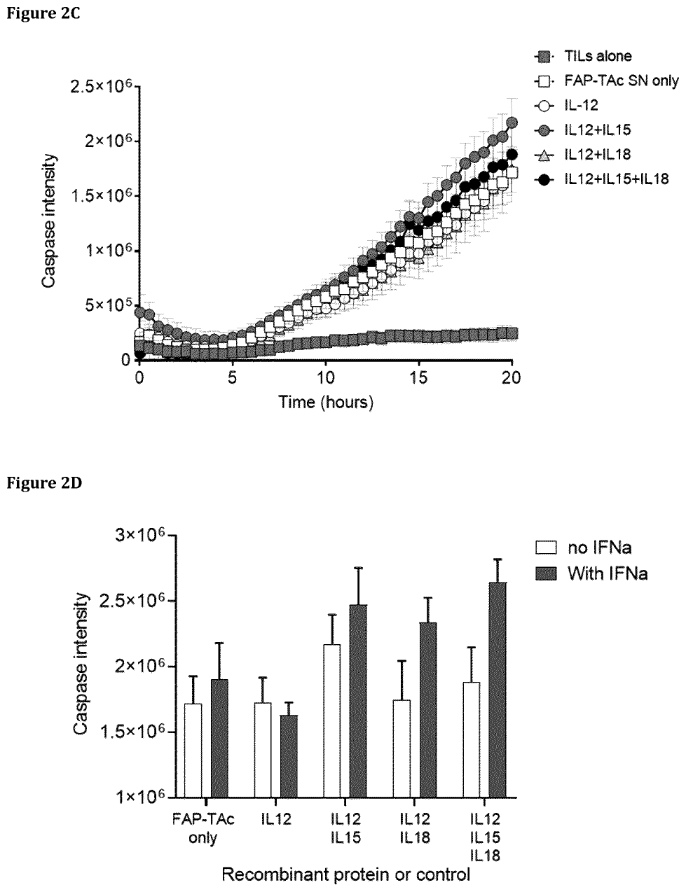

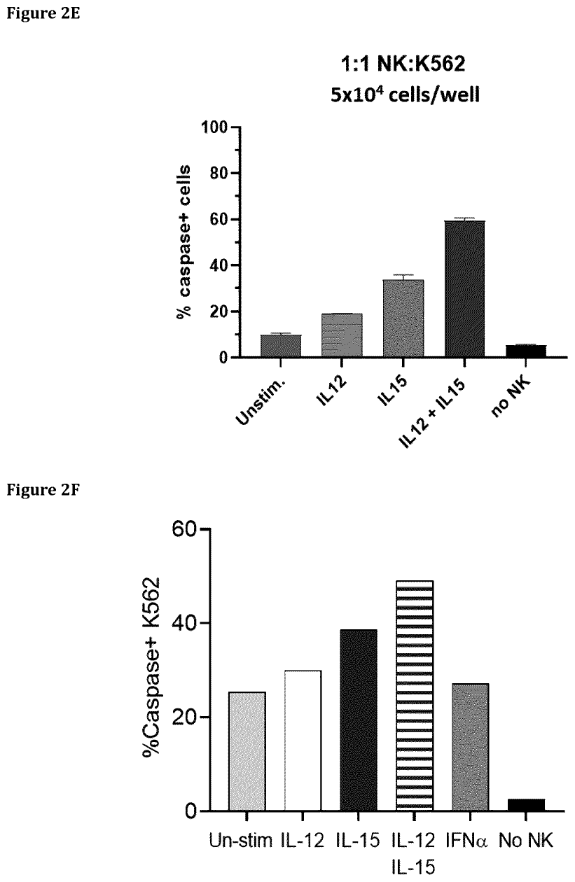

A549 cells were infected with 1ppc NG-617, which expresses the FAP-TAc (also described in the literature as a bispecific T-cell engager, BiTE). Supernatants were harvested 11 days post infection and clarified by centrifugation at 300×g for 5 minutes and then aliquoted and frozen at −80° C. The FAP expressing lung fibroblast cell line MRC-5 was seeded into 96 well plates at a density of 1×10⁴ cells per well which were incubated at 37° C., 5% CO₂ for 24 hours before staining with 5μM Caspase Green reagent (IncuCyte; Essen Bioscience). Media was removed and replaced with freshly thawed NG-617 treated cell supernatant containing FAP-TAc protein, along with T cells isolated from human PBMC donors at 6×10⁵ cells per well, and recombinant IL-12 p70 (15 ng/mL, R&D Systems) was added, either alone or in combination with IL-18 (50 ng/mL, R&D Systems), IL-15 (50 ng/mL, InvivoGen) and/or IFNα(1,500U/mL, InvivoGen). Plates were then incubated at 37° C., 5% CO₂ for 24 hours in an IncuCyte live cell imager taking 4 images per well every 30 minutes. Data showed that IL-12 alone did not enhance FAP-TAc mediated T cell killing of MRC5 cells above that stimulated by FAP-TAc alone (observed as an increase in caspase positive MRC5 cells), while IL-12 and IL-15 together with or without IL-18 providing enhancement of killing (FIG. 2A). In the same experiment, IFNα further enhanced T-cell mediated killing with or without added cytokines (FIG. 2B).

In a second experiment, CD45+tumour infiltrating leukocytes (TILs) were isolated from tumour 70 using a TIL isolation kit (Miltenyi) on an AutoMACS cell isolator (Miltenyi). A T-cell mediated killing assay was then carried out as described above, using TILs in place of PBMC-derived T cells. Data showed that IL-12 alone did not enhance T cell killing of MRC5 cells above the FAP-TAc alone while IL-12+IL-15 and IL-12+IL-15+IL-18 did enhance killing, with IL-12+IL-15 providing the greatest enhancement (FIG. 2C). In the same experiment, IFNα enhanced killing when added to any of the combinations except IL-12 alone (FIG. 2D).

In another experiment, NK cells were isolated from PBMCs and pre-stimulated with IL-12 and/or IL-15 for 24 hours and then rested for 12 hours before incubating them with the low MHC class I expressing K562 cell line. K562 cells (target cells) were pre-labelled with Violet Cell tracker (ThermoFisher) according to the manufacturers protocol and then incubated at 37° C., 5% CO₂ with NK cells at a 1:1 ratio (5×10⁴ cells per well in a 96-well plate) in the presence of 5μM Caspase Green reagent for 3 hours. Cells were then spun at 300×g for 5 minutes and washed twice with PBS and resuspended in flow buffer. Samples were analysed by flow cytometry using an Attune NxT flow cytometer and percentage of caspase-positive K562 cells were plotted (FIG. 2E). Data showed that both IL-12 and IL_15 can individually enhance NK-mediated killing, which is further increased by combining these two cytokines.

In a similar experiment, K562 cells were incubated as described above with TILs derived from a primary kidney tumour. Dissociated tumour cells containing ˜16% NK as a proportion of total live cells were seeded in a collagen-coated 24-well plate and after 4 hours non-adherent cells (TIL-enriched cells) was transferred to another collagen-coated plate and stimulated or not with the indicated cytokines for 18 hours. Cells were then counted and incubated with labelled K562 target cells for 4 hours (ratio effectors:target cells 2:1 based on NK numbers at day 0). Primary tumour-infiltrating NK cells stimulated with IL-12 or IL-15 alone showed enhanced killing of K562 target cells, with a combination of the two cytokines giving a greater enhancement of killing (FIG. 2F).

### EXAMPLE 3: Migration of Immune Cells in Response to Recombinant Chemokines

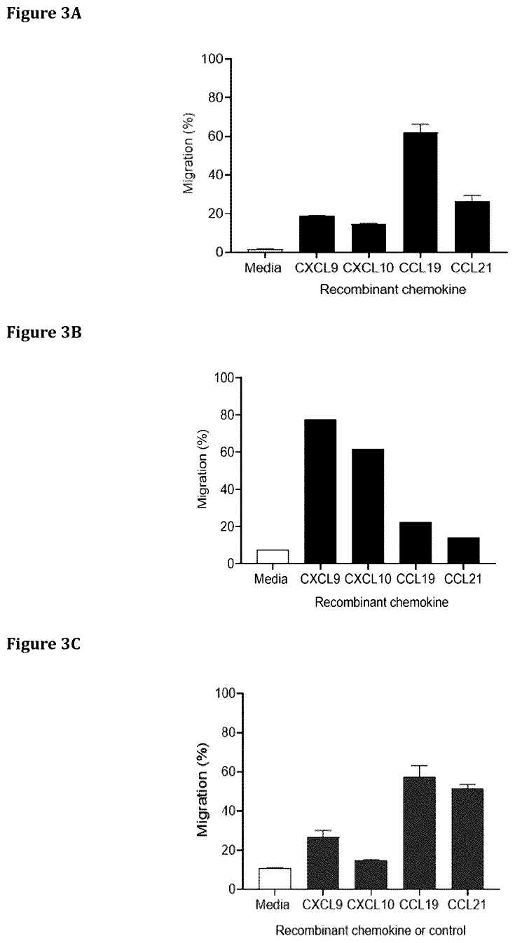

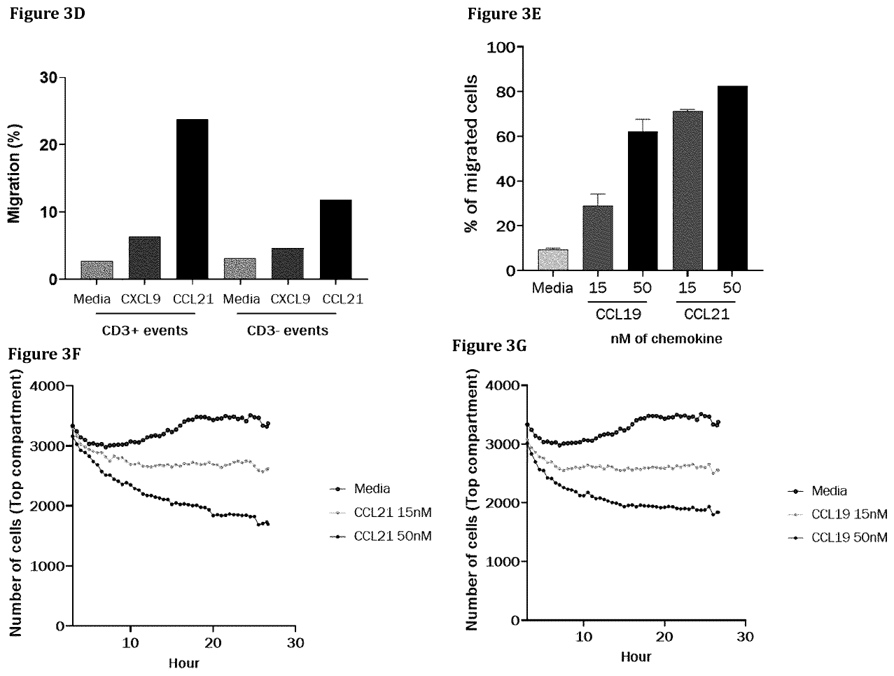

Several experiments were carried out to evaluate chemokine effects on the migration of the immune cell subsets in PBMC preparations as well as TILs isolated from primary tumour samples. Migration assays were carried out using the Transwell assay system (Corning), whereby chemokines were added to media in a 96 or 24 well plate, before adding a plate insert on top with a permeable membrane containing holes of 3 μm in diameter, into which the cells were added. T cells and their subsets were magnetically isolated from PBMCs using MACS beads (Miltenyi), and TILs were isolated from tumour samples dissociated as described in Example 1. Media either alone, or containing CXCL9, CXCL10, CCL19 or CCL21 at 50 nM, was added into the bottom of Transwell plates, before adding naïve T cells (100,000 cells per well) or effector CD8+ T cells (80,000 cells per well) into the upper compartment Plates were incubated at 37° C., 5% CO2 for 3 hours and 30 minutes before counting the cells in the lower compartment by flow cytometry using an Attune NxT (Thermofisher). All chemokines stimulated migration of both naïve and effector T-cells above the medium only control, but more naïve T cells were stimulated to migrate by CCL19 and CCL21 than by CXCL9 or CXCL10 (FIG. 3A), and the inverse was true for effector T cells, with CXCL9 and CXCL10 stimulated cells migrating in larger numbers (FIG. 3B).

In a second experiment, TILs isolated from a breast tumour (tumour 53) were rested for approximately 24h and then added to Transwell plates as described previously. All four chemokines stimulated migration above background (media only control), with CCL19 and CCL21 stimulating the migration of the largest number of TILs (FIG. 3C).

In a third experiment, leukocytes from dissociated primary lymph nodes removed as part of breast cancer surgery (same procedure used for primary tumours and described above in Example 1) were left to adhere on plastic in a flask (at 37° C., 5% C02) for 3 hours before removing non-adherent cells for the migration assay. A transwell migration assay was performed as described above in a 24-well plate, where 1.2×10⁵ cells were added to the upper compartment. Each condition was run in duplicate and cells in the bottom compartment after migration were counted and stained/analysed by flow cytometry using an Attune NxT (Thermofisher)(FIG. 3D). Data showed that both CXCL9 and CCL21 induced migration of lymph node-derived T cells (CD3+) and non-T cells (CD3-lymphocytes).

In another experiment, monocyte-derived dendritic cells from a healthy donor were prepared by culturing them with 50 ng/mL of recombinant GM-CSF plus IL-4 for 7 days and then matured with LPS for 24h before testing them in migration assays. Transwell assay was performed using 24-well plates, with inserts whose permeable membrane contains holes of 8μm in diameter, which were previously coated with 100 μg/mL of Matrigel. Migration assay were then run for 6 hours at 37° C., 5% CO2 and analysed as described above. As shown in FIG. 3E, monocyte-derived dendritic cells strongly respond to both CCL19 and CCL21. An Incucyte chemotaxis assay was also performed following the manufacture's protocol using 96-well plates (Essen Bioscience) with 8μm-pore membrane coated with Matrigel (FIG. 3F). Cells on top of the membrane were scanned and quantified over time using the Chemotaxis analysis software. As shown in FIG. 3F, dendritic cell numbers in the top compartment decreased over time in response to recombinant CCL19 or CCL21 compared to media control, demonstrating their chemotactic response to these chemokines.

### EXAMPLE 4: Cytokine-Mediated Enhancement of Tumour Ag-Specific Responses by Lymph Node Derived T-Cells from Breast Cancer Surgery

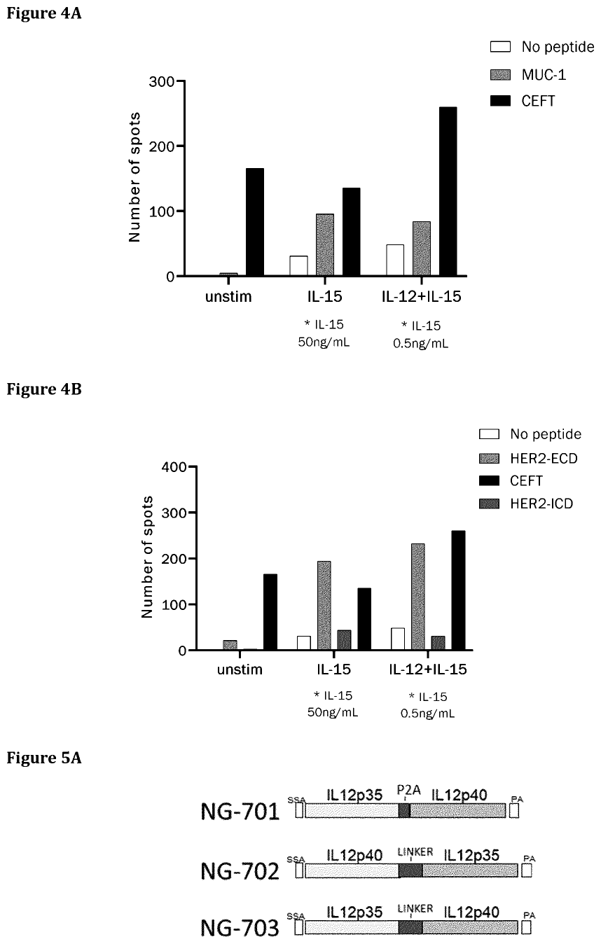

To assess if IL-12 and IL-15 cytokines can enhance an antigen-specific anti-tumour T cell response, an IFNγ ELISpot assay with primary lymph nodes derived cells from breast cancer surgery was performed. After dissociation (as described above in Example 3), lymph node cells were seeded in a U-bottom 96-well plate and treated with either breast cancer-associated peptide pools, or CEFT (Clostridium tetani, Epstein-Barr virus (HHV-4), Human cytomegalovirus (HHV-5), Influenza A) peptide pool or just DMSO-containing media as controls. The following breast cancer-associated peptide pools (purchased from JPT) were used in this assay: MUC-1 (mucin-1), HER2-ECD (receptor tyrosine-protein kinase erbB-2-extracellular domain) and HER2-ICD (receptor tyrosine-protein kinase erbB-2-intracellular domain). After 1 hour, recombinant IL-12 or IL-15, or combination of those cytokines, was added to the cultures at the indicated concentrations and cells were left for 6 days at 37° C., 5% CO2. The day before the IFNγ ELISpot assay, cells were rested (by removing cell culture media and replacing it just with complete media). On the day of ELISpot, cells were counted and plated (2.5×10⁴ per well) and boosted with the same peptides they were stimulated with. Also, at the same time, previously frozen cells (on the day of lymph node dissociation) from the same sample were thawed and added (1.85×10⁵ per well) as antigen presenting cells. IFNγ ELISpot plates were developed according to the manufacture's protocol (Mabtech) and then spot numbers read using an ELISpot plate reader (CTL Europe GmbH). As shown in FIG. 4, IL-15 significantly increased T-cell responses to MUC-1 (FIG. 4A) and HER2 (FIG. 4B).

### EXAMPLE 5: Production of Viruses Encoding Human IL-12 p35 and IL-12 p40 Either as Separate Proteins or Joined by a Flexible Linker

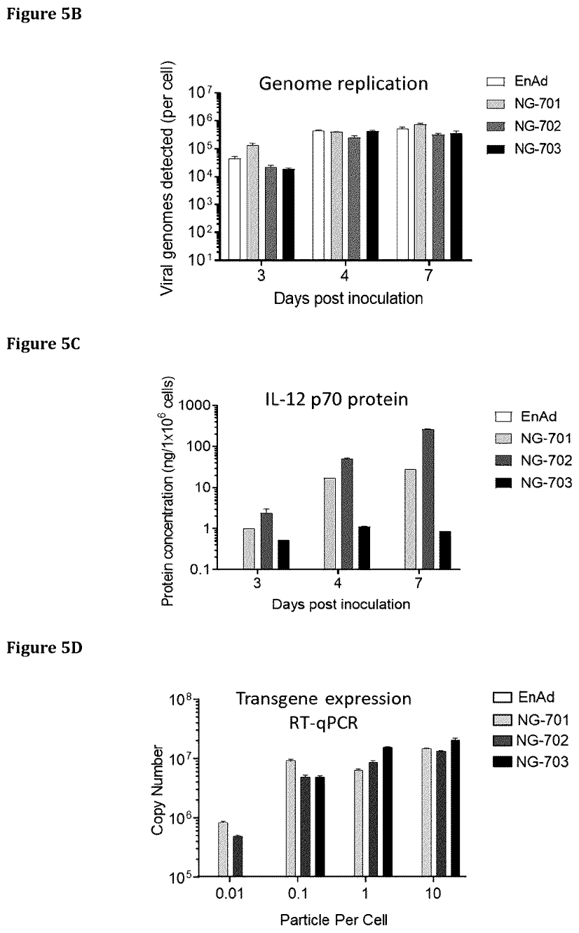

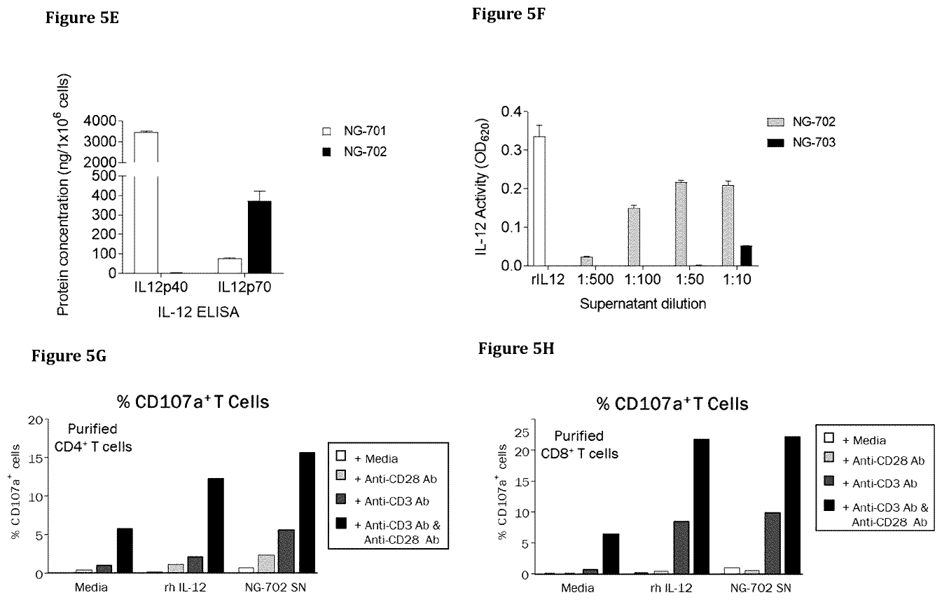

Three viruses (NG-701, NG-702, NG-703) were generated that differently encode human IL-12 transgenes (Table 1, FIG. 5A).

Table 1

| Virus ID | Transgene Cassette |
|---|---|
| NG-701 | SSA¹-IL12p35²-P2A³-IL12p40⁴-PA⁵ |
| (SEQ ID NO: 74) | (SEQ ID NO: 233) |
| NG-702 | SSA¹-IL12p40LinkerIL12p35⁶-PA⁵ |
| (SEQ ID NO: 75) | (SEQ ID NO: 234) |
| NG-703 | SSA¹-IL12p35LinkerIL12p40⁷-PA⁵ |
| (SEQ ID NO: 76) | (SEQ ID NO: 235) |

¹SEQ ID NO. 1; ²SEQ ID NO. 9; ³SEQ ID NO. 4; ⁴SEQ ID NO. 10; ⁵SEQ ID NO. 8; ⁶SEQ ID NO. 11; ⁷SEQ ID NO. 12;

In each transgene cassette, the cDNA encoding the IL-12 sequences was flanked at the 5′ end with a short splice acceptor sequence (SSA, SEQ ID NO: 1-CAGG). At the 3′ end of the IL-12 sequences, a SV40 late poly(A) sequence (PA, SEQ ID NO: 8) was encoded. In virus NG-701 the individual IL-12 p35 and IL-12 p40 sequences were linked with a P2A ribosome skipping sequence (SEQ ID NO: 4) to enable both IL-12 chains to be translated and produced as separate chains. In viruses NG-702 and NG-703 the IL-12 transgene encoded a single chain variant created by linking the sequences for the two individual p35 and p40 IL-12 chains with a sequence encoding a flexible linker (Gly₄Ser).

### Virus Production

The plasmid pColoAd2.4 (WO2015/097220) was used to generate the plasmids pNG-701, pNG-702 and pNG-703 by direct insertion of synthesised transgene cassettes encoding the transgene proteins. The pColoAd2.4 plasmid and transgene cassette were digested using AsiSI and SbfI restriction enzymes. Each digested transgene cassette was directly ligated into the digested pColoAd2.4 plasmid. The pNG-701 transgene cassette encodes for IL-12 p35 and IL-12 p40 as two separate transgene proteins (SEQ IDs NOs: 9 and 10), the pNG-702 transgene cassette encodes a single chain IL-12 molecule (SEQ ID NO. 11) comprising the IL-12 p40 protein covalently linked to the N-terminus of the IL-12 p35 protein with a (Gly₄Ser)₃ linker (SEQ ID NO: 31) and the pNG-703 transgene cassette encodes a single chain IL-12 molecule (SEQ ID NO: 12) comprising the IL-12 p35 protein covalently linked to the N-terminus of the IL-12 p40 protein with a (Gly₄Ser)₃ linker. Schematics of the transgene cassettes are shown in FIG. 5A. Construction of plasmid DNA was confirmed by restriction analysis and Sanger sequencing.

The plasmids, pNG-701, pNG-702 and pNG-703 were linearised by restriction digest with the enzyme AscI to produce the virus genomes. The viruses were amplified and purified according to methods given below.

Digested DNA was purified by phenol/chloroform extraction and precipitated for 16±2 hrs, −20° C. in 600 μl >95% molecular biology grade ethanol and 15 μl 3M Sodium Acetate. The precipitated DNA was pelleted by centrifuging at 13000 rpm, 5 mins and was washed twice in 500 μl 70% ethanol. The clean DNA pellet was air dried, resuspended in 500 μl OptiMEM containing 15 μl lipofectamine transfection reagent and incubated for 30 mins, RT. The transfection mixture was then added drop wise to a T-25 flask containing 293 cells grown to 70% confluency. After incubation of the cells with the transfection mix for approximately 2 hrs at 37° C., 5% CO₂ 4mls of cell media (DMEM high glucose with glutamine supplemented with 2% FBS) was added to the cells and the flasks was incubated 37° C., 5% CO₂.

The transfected 293 cells were monitored every 24 hrs and were supplemented with additional media as required. The production of virus was monitored by observation of a significant cytopathic effect (CPE) in the cell monolayer. Once extensive CPE was observed the virus was harvested from 293 cells by three freeze-thaw cycles. The harvested viruses were used to re-infect 293 cells in order to amplify the virus stocks. Viable virus production during amplification was confirmed by observation of significant CPE in the cell monolayer. Once CPE was observed the virus was harvested from 293 cells by three freeze-thaw cycles. The amplified stocks of viruses were used for further amplification before the viruses were purified by double caesium chloride banding to produce purified virus stocks.

Viral Particle Infectivity & IL-12 p70 Quantification

A549 human lung adenocarcinoma cells were seeded in 12 well plates at a cell density of 7.5×10⁵ cells/well and infected with either EnAd, NG-701, NG-702 or NG-703 at 0.01 particles per cell (ppc). Plates were incubated at 37° C., 5% CO₂ before harvesting cells and supernatants 3,4 or 7 days later. Supernatant samples were clarified by centrifugation at 300×g for 5 minutes. The cell fraction was obtained by adding RLT lysis buffer (Qiagen)+beta-mercaptoethanol (Sigma) to each well and pipetting to ensure maximal recovery. The cell fraction was then pooled with the pellet obtained during supernatant clarification and viral genomes were quantified for each time point by qPCR. Data demonstrated that each of the tested viruses produced similar quantities of viral genomes with similar kinetics (FIG. 5B). IL-12 p70 protein was quantified by ELISA from supernatant samples from the same experiment. ELISA data showed that all three viruses encoding IL-12 produced IL-12 p70 protein, with NG-701 and NG-702 producing more than NG-703 (FIG. 5C). A similar second study, using different ppc levels, RT-qPCR analysis demonstrated similar transgene mRNA expression by NG-701, NG-702 and NG-703 (FIG. 5D).

In a third experiment, A549 human lung adenocarcinoma cells were seeded in T175 flasks and infected with 10ppc of NG-701 or NG-702 at a cell density of 1.45×10⁷ cells/flask. Flasks were incubated at 37° C., 5% CO₂ before harvesting cells and supernatant 72 hours later. Cells and supernatant were separated by centrifugation at 300×g, as described above. IL-12 p40 and IL-12 p70 proteins were quantified in supernatant samples by ELISA. Data showed that NG-701, encoding IL-12 p40 and IL-12 p35 as separate proteins, predominantly produced the IL-12 p40 protein with little IL-12 p70, while NG-702, encoding the two IL-12 subunits joined by a flexible linker, produced primarily the IL-12 p70 protein (FIG. 5E).

In another experiment, A549 human lung adenocarcinoma cells were seeded in T175 flasks and infected with 10ppc of NG-702 or NG-703 at a cell density of 2.6×10⁷ cells/flask. Flasks were incubated at 37° C., 5% CO₂ before harvesting supernatant 72 hours later. Cells and supernatant were separated by centrifugation at 300×g, as described above. The functional activity of the produced IL-12 p70 protein was assessed using a HEK-Blue reporter cell line. HEK-Blue™ IL-12 (InvivoGen) cells stably express the human IL-12 receptor and genes of the IL-12 signalling pathway along with a STAT4-inducible secreted alkaline phosphatase (SEAP) reporter gene. HEK-Blue IL-12 cells were seeded at 5×10⁴ cells per well in 96 well plates before being stimulated with supernatants or recombinant IL-12 (InvivoGen) at 100 ng/mL, followed by incubation at 37° C., 5% CO2 for 18-24 hours. Assay plates were centrifuged at 300×g for 5 minutes before removing clarified supernatant and transferring 20 μL into a separate 96 well plate along with 180 μL of Quanti-Blue reagent. The plate was incubated for 1 hour at 37° C. before analysing the plate on SpectraMax i3x plate reader with absorbance set to 620 nm. Data showed that supernatant from NG-702 infected cells led to the secretion of more SEAP from the reporter cells than supernatants from NG-703 infected cells (FIG. 5F).

In a further study, purified human CD4+ or CD8+ T-cells were activated with anti-CD3, anti-CD28 or both anti-CD3 and anti-CD28, or left unactivated, and cultured in the presence of recombinant human IL-12 (rhIL-12) or supernatants (SN) from NG-702 infected A549 cells for 6 days. Cells were then harvested and analysed by flow cytometry for the expression of NG-107a as a measure activation of cytotoxic T-cell effector function. NG-702 supernatants enhanced the expression of CD107a to a similar degree to that seen with rhIL-12 (FIGS. 5G&H).

### EXAMPLE 6: Production of Viruses Encoding a Single Chain IL-12 Together with Other Transgenes

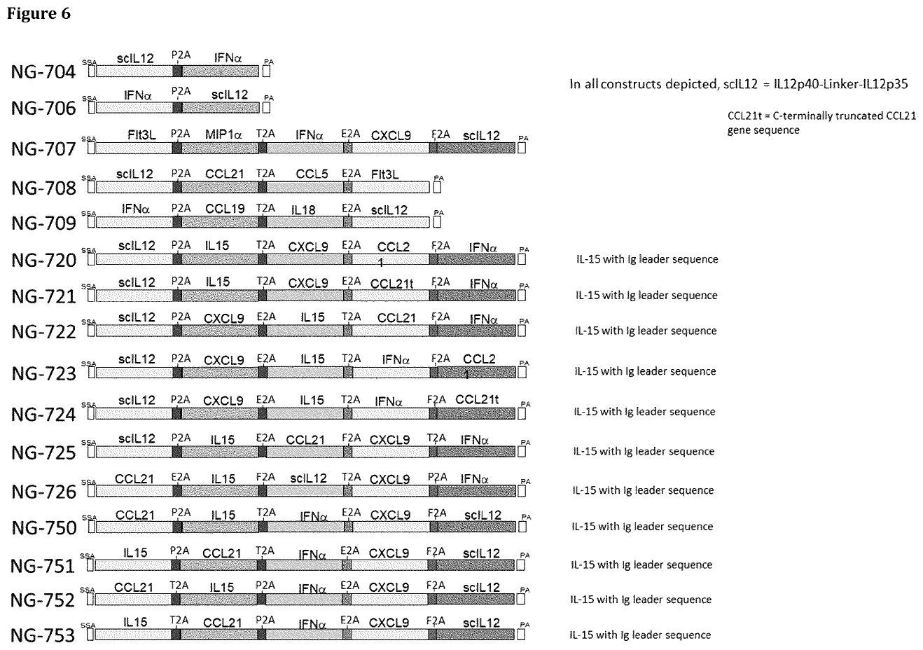

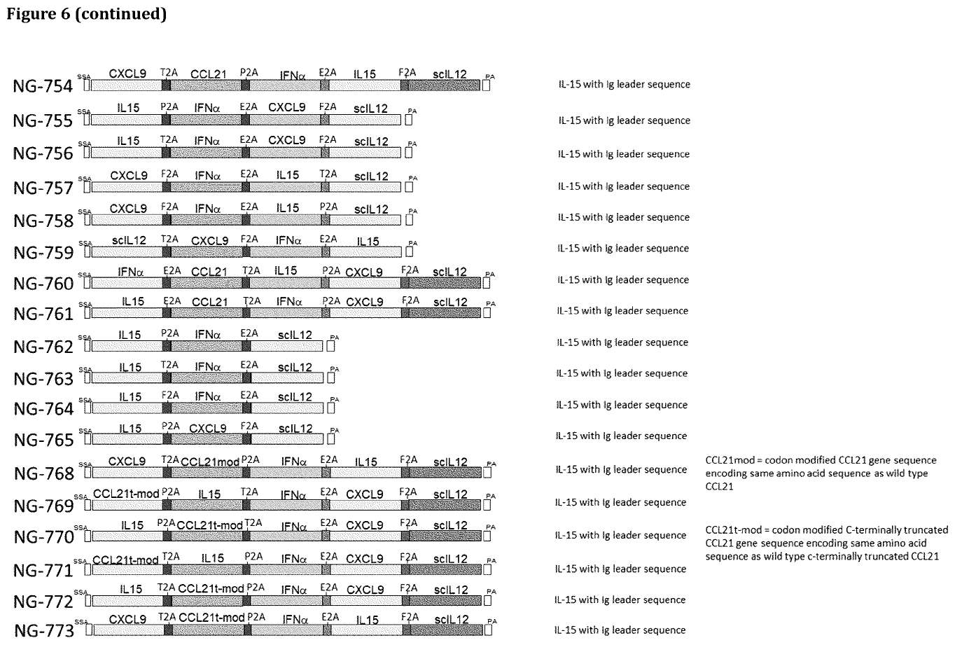

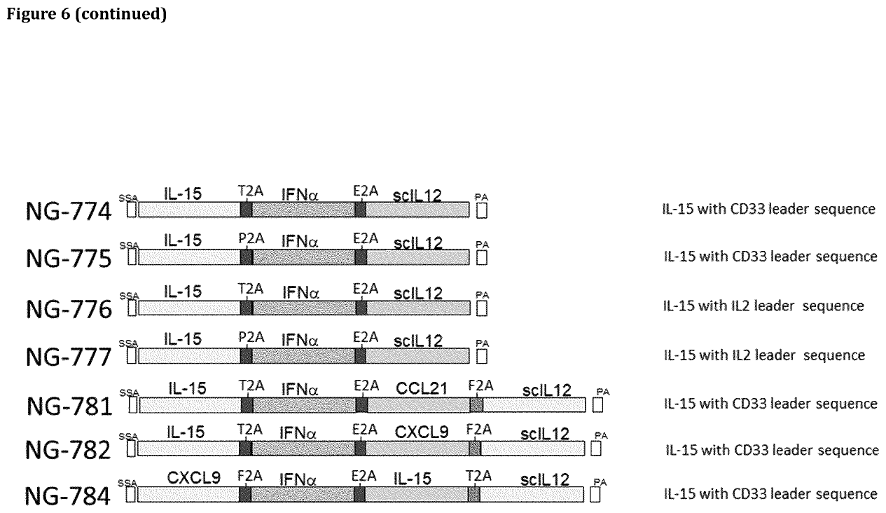

A set of viruses with transgene cassettes comprising a single chain IL-12 transgene as well as one or more additional transgenes were designed, produced and purified. Viruses NG-704, NG-706, NG-707, NG-708 and NG-709 were generated according to the methods of Example 5. For the remaining viruses (NG-720 and higher numbers), the pColoAd2.4 plasmid was digested using AsiSI and SbfI restriction enzymes and each synthesised transgene cassette was amplified by PCR using primers to add a 20 bp sequence to the 5′ and the 3′ ends of the amplified PCR product. The added sequences were complementary to sequences flanking the transgene cassette insertion site of the pColoAd2.4 plasmid and enabled direct assembly of the PCR amplified transgene cassette into the digested pColoAd2.4 plasmid. Subsequent steps were the same as described in Example 5. The viruses encoding a single chain IL-12 plus at least one other transgene are listed in Table 2 and illustrated in FIG. 6. For some virus preparations, smaller scale purifications were run using Optiprep (Iodixanol) density gradients, centrifuging at 155,000 g for 1 hour at 10° C., instead of using caesium chloride.

TABLE 2
Viruses with transgene cassettes having a single chain IL-12
(IL12p40LinkerIL12p35) plus one or more other transgenes

| Virus ID | Transgene Cassette |
|---|---|
| NG-704 (SEQ ID NO. 77) | SSA¹-IL12p40LinkerIL12p35⁶-P2A³-IFNa⁸-PA⁵ (SEQ ID NO. 36) |
| NG-706 (SEQ ID NO. 78) | SSA¹-IFNa⁸-P2A³-IL12p40LinkerIL12p35⁶-PA⁵ (SEQ ID NO. 37) |
| NG-707 (SEQ ID NO. 79) | SSA¹-Flt3L⁹-P2A³-MIP1a¹⁰-T2A¹¹-IFNa⁸-E2A¹²-CXCL9¹³-F2A¹⁴- |
| | IL12p40Linkerp35⁶-PA⁵ (SEQ ID NO. 38) |
| NG-708 (SEQ ID NO. 80) | SSA¹-IL12p40Linkerp35⁶-P2A³-CCL21¹⁵-T2A¹¹-CCL5¹⁶-E2A¹²- |
| | Flt3L⁹-PA⁵ (SEQ ID NO. 39) |
| NG-709 (SEQ ID NO. 81) | SSA¹-IFNa⁸-P2A³-CCL19¹⁷-T2A¹¹-IL18¹⁸-E2A¹²- |
| | IL12p40LinkerIL12p35⁶-PA⁵ (SEQ ID NO. 40) |
| NG-720 (SEQ ID NO. 82) | SSA¹-IL12p40LinkerIL12p35⁶-P2A³-IL15¹⁹-T2A¹¹- CXCL9¹³-E2A¹²- |
| | CCL21¹⁵-F2A¹⁴-IFNa⁸-PA⁵ (SEQ ID NO. 41) |
| NG-721 (SEQ ID NO. 83) | SSA¹-IL12p40LinkerIL12p35⁶-P2A³-IL15¹⁹-T2A¹¹- CXCL9¹³-E2A¹²- |
| | CCL21trunc²⁰-F2A¹⁴-IFNa⁸-PA⁵ (SEQ ID NO. 42) |
| NG-722 (SEQ ID NO. 84) | SSA¹-IL12p40LinkerIL12p35⁶-P2A³-CXCL9¹³-E2A¹²-IL15¹⁹-T2A¹¹- |
| | CCL21¹⁵-F2A¹⁴-IFNa⁸-PA⁵ (SEQ ID NO. 43) |
| NG-723 (SEQ ID NO. 85) | SSA¹-IL12p40LinkerIL12p35⁶-P2A³-CXCL9¹³-E2A¹²-IL15¹⁹-T2A¹¹- |
| | IFNa⁸-F2A¹⁴-CCL21¹⁵-PA⁵ (SEQ ID NO. 44) |
| NG-724 (SEQ ID NO. 86) | SSA¹-IL12p40LinkerIL12p35⁶-P2A³-CXCL9¹³-E2A¹²-IL15¹⁹-T2A¹¹- |
| | IFNa⁸-F2A¹⁴-CCL21trunc²⁰-PA⁵ (SEQ ID NO. 45) |
| NG-725 (SEQ ID NO. 87) | SSA¹-IL12p40LinkerIL12p35⁶-P2A³-IL15¹⁹-E2A¹²-CCL21¹⁵-F2A¹⁴- |
| | CXCL9¹³-T2A¹¹-IFNa⁸-PA⁵ (SEQ ID NO. 46) |
| NG-726 (SEQ ID NO. 88) | SSA¹-CCL21¹⁵-E2A¹²-IL15¹⁹-F2A¹⁴-IL12p40LinkerIL12p35⁶-T2A¹¹- |
| | CXCL9¹³-P2A³-IFNa⁸-PA⁵ (SEQ ID NO. 47) |
| NG-750 (SEQ ID NO. 93) | SSA¹-CCL21¹⁵-P2A³-IL15¹⁹-T2A¹¹-IFNa⁸-E2A¹²-CXCL9¹³-F2A¹⁴- |
| | IL12p40LinkerIL12p35⁶-PA⁵ (SEQ ID NO. 52) |
| NG-751 (SEQ ID NO. 94) | SSA¹-IL15¹⁹-P2A³-CCL21¹⁵-T2A¹¹-IFNa⁸-E2A¹²-CXCL9¹³-F2A¹⁴- |
| | IL12p40LinkerIL12p35⁶-PA⁵ (SEQ ID NO. 53) |
| NG-752 (SEQ ID NO. 95) | SSA¹-CCL21¹⁵-T2A¹¹-IL15¹⁹-P2A³-IFNa⁸-E2A¹²-CXCL9¹³-F2A¹⁴- |
| | IL12p40LinkerIL12p35⁶-PA⁵ (SEQ ID NO. 54) |
| NG-753 (SEQ ID NO. 96) | SSA¹-IL15¹⁹-T2A¹¹-CCL21¹⁵-P2A³-IFNa⁸-E2A¹²-CXCL9¹³-F2A¹⁴- |
| | IL12p40LinkerIL12p35⁶-PA⁵ (SEQ ID NO. 55) |
| NG-754 (SEQ ID NO. 97) | SSA¹-CXCL9¹³-T2A¹¹-CCL21¹⁵-P2A³-IFNa⁸-E2A¹²-IL15¹⁹-F2A¹⁴- |
| | IL12p40LinkerIL12p35⁶-PA⁵ (SEQ ID NO. 56) |
| NG-755 (SEQ ID NO. 98) | SSA¹-IL15¹⁹-P2A³-IFNa⁸-E2A¹²-CXCL9¹³-F2A¹⁴- |
| | IL12p40LinkerIL12p35⁶-PA⁵ (SEQ ID NO. 57) |
| NG-756 (SEQ ID NO. 99) | SSA¹-IL15¹⁹-T2A¹¹-IFNa⁸-E2A¹²-CXCL9¹³-F2A¹⁴- |
| | IL12p40LinkerIL12p35⁶-PA⁵ (SEQ ID NO. 58) |
| NG-757 (SEQ ID NO. 100) | SSA¹-CXCL9¹³-F2A¹⁴-IFNa⁸-E2A¹²-IL15¹⁹-T2A¹¹- |
| | IL12p40LinkerIL12p35⁶-PA⁵ (SEQ ID NO. 59) |
| NG-758 (SEQ ID NO. 101) | SSA¹-CXCL9¹³-F2A¹⁴-IFNa⁸-E2A¹²-IL15¹⁹-P2A³- |
| | IL12p40LinkerIL12p35⁶-PA⁵ (SEQ ID NO. 60) |
| NG-759 (SEQ ID NO. 102) | SSA¹-IL12p40LinkerIL12p35⁶-T2A¹¹-CXCL9¹³-F2A¹⁴-IFNa⁸-E2A¹²- |
| | IL15¹⁹-PA⁵ (SEQ ID NO. 61) |
| NG-760 (SEQ ID NO. 103) | SSA¹-IFNa⁸-E2A¹²-CCL21¹⁵-T2A¹¹-IL15¹⁹-P2A³-CXCL9¹³-F2A¹⁴- |
| | IL12p40LinkerIL12p35⁶-PA⁵ (SEQ ID NO. 62) |
| NG-761 (SEQ ID NO. 104) | SSA¹-IL15¹⁹-E2A¹²-CCL21¹⁵-T2A¹¹-IFNa⁸-P2A³-CXCL9¹³-F2A¹⁴- |
| | IL12p40LinkerIL12p35⁶-PA⁵ (SEQ ID NO. 63) |
| NG-762 (SEQ ID NO. 105) | SSA¹-IL15¹⁹-P2A³-IFNa⁸-E2A¹²-IL12p40LinkerIL12p35⁶-PA⁵ |
| | (SEQ ID NO. 64) |
| NG-763 (SEQ ID NO. 106) | SSA¹-IL15¹⁹-T2A¹¹-IFNa⁸-E2A¹²-IL12p40LinkerIL12p35⁶-PA⁵ |
| | (SEQ ID NO. 65) |
| NG-764 (SEQ ID NO. 107) | SSA¹-IL15¹⁹-F2A¹⁴-IFNa⁸-E2A¹²-IL12p40LinkerIL12p35⁶-PA⁵ |
| | (SEQ ID NO. 66) |
| NG-765 (SEQ ID NO. 108) | SSA¹-IL15¹⁹-P2A³-CXCL9¹³-F2A¹⁴-IL12p40LinkerIL12p35⁶-PA⁵ |
| | (SEQ ID NO. 67) |
| NG-768 (SEQ ID NO. 109) | SSA¹-CXCL9¹³-T2A¹¹-CCL21mod²³-P2A³-IFNa⁸-E2A¹²-IL15¹⁹-F2A¹⁴- |
| | IL12p40LinkerIL12p35⁶-PA⁵ (SEQ ID NO. 68) |
| NG-769 (SEQ ID NO. 110) | SSA¹-CCL21tmod²⁴-P2A³-IL15¹⁹-T2A¹¹-IFNa⁸-E2A¹²-CXCL9¹³- |
| | F2A¹⁴-IL12p40LinkerIL12p35⁶-PA⁵ (SEQ ID NO. 69) |
| NG-770 (SEQ ID NO. 111) | SSA¹-IL15¹⁹-P2A³-CCL21tmod²⁴-T2A¹¹-IFNa⁸-E2A¹²-CXCL9¹³- |
| | F2A¹⁴-IL12p40LinkerIL12p35⁶-PA⁵ (SEQ ID NO. 70) |
| NG-771 (SEQ ID NO. 112) | SSA¹-CCL21tmod²⁴-T2A¹¹-IL15¹⁹-P2A³-IFNa⁸-E2A¹²-CXCL9¹³- |
| | F2A¹⁴-IL12p40LinkerIL12p35⁶-PA⁵ (SEQ ID NO. 71) |
| NG-772 (SEQ ID NO. 113) | SSA¹-IL15¹⁹-T2A¹¹-CCL21tmod²⁴-P2A³-IFNa⁸-E2A¹²-CXCL9¹³-F2A⁹- |
| | IL12p40LinkerIL12p35⁶-PA⁵ (SEQ ID NO. 72) |
| NG-773 (SEQ ID NO. 114) | SSA¹-CXCL9¹³-T2A¹¹-CCL21tmod²⁴-P2A³-IFNa⁸-E2A¹²-IL15¹⁹- |
| | F2A¹⁴-IL12p40LinkerIL12p35⁶-PA⁵ (SEQ ID NO. 73) |
| NG-774 (SEQ ID No. 212) | SSA¹-IL15²⁵-T2A¹¹-IFNa⁸-E2A¹²-IL12p40LinkerIL12p35⁶-PA⁵ |
| | (SEQ ID NO. 168) |
| NG-775 (SEQ ID NO. 213) | SSA¹-IL15²⁵-P2A³-IFNa⁸-E2A¹²-IL12p40LinkerIL12p35⁶-PA⁵ |
| | (SEQ ID NO. 169) |
| NG-776 (SEQ ID NO. 214) | SSA¹-IL15²⁶-T2A¹¹-IFNa⁸-E2A¹²-IL12p40LinkerIL12p35⁶-PA⁵ |
| | (SEQ ID NO. 170) |
| NG-777 (SEQ ID NO. 215) | SSA¹-IL15²⁶-P2A³-IFNa⁸-E2A¹²-IL12p40LinkerIL12p35⁶-PA⁵ |
| | (SEQ ID NO. 171) |
| NG-781 (SEQ ID NO. 216) | SSA¹-IL15²⁵-T2A¹¹-IFNa⁸-E2A¹²-CCL21²⁸-F2A⁹- |
| | IL12p40LinkerIL12p35⁶-PA⁵ (SEQ ID NO. 172) |
| NG-782 (SEQ ID NO. 217) | SSA¹-IL15²⁵-T2A¹¹-IFNa⁸-E2A¹²-CXCL9¹³-F2A⁹- |
| | IL12p40LinkerIL12p35⁶-PA⁵ (SEQ ID NO. 173) |
| NG-784 (SEQ ID NO. 218) | SSA¹-CXCL9¹³-F2A⁹-IFNa⁸-E2A¹²-IL15²⁵-T2A¹¹- |
| | IL12p40LinkerIL12p35⁶-PA⁵ (SEQ ID NO. 174) |

¹SEQ ID NO. 1; ³SEQ ID NO. 4; ⁵SEQ ID NO. 8; ⁶SEQ ID NO. 11; ⁸SEQ ID NO. 13; ⁹SEQ ID NO. 14; ¹⁰SEQ ID NO. 15; ¹¹SEQ ID NO. 5; ¹²SEQ ID NO. 6; ¹³SEQ ID NO. 16; ¹⁴SEQ ID NO. 7; ¹⁵SEQ ID NO. 17; ¹⁶SEQ ID NO. 18; ¹⁷SEQ ID NO. 19; ¹⁸SEQ ID NO. 20; ¹⁹SEQ ID NO. 21; ²⁰SEQ ID NO. 24; ²³SEQ ID NO. 33; ²⁴SEQ ID NO. 35; ²⁵SEQ ID NO. 163; ²⁶SEQ ID NO. 165;

### EXAMPLE 7: Production and Activity of Viruses Encoding Human IL-12 and Other Transgenes

### Viral Particle Infectivity & Quantification of Transgene Protein Production

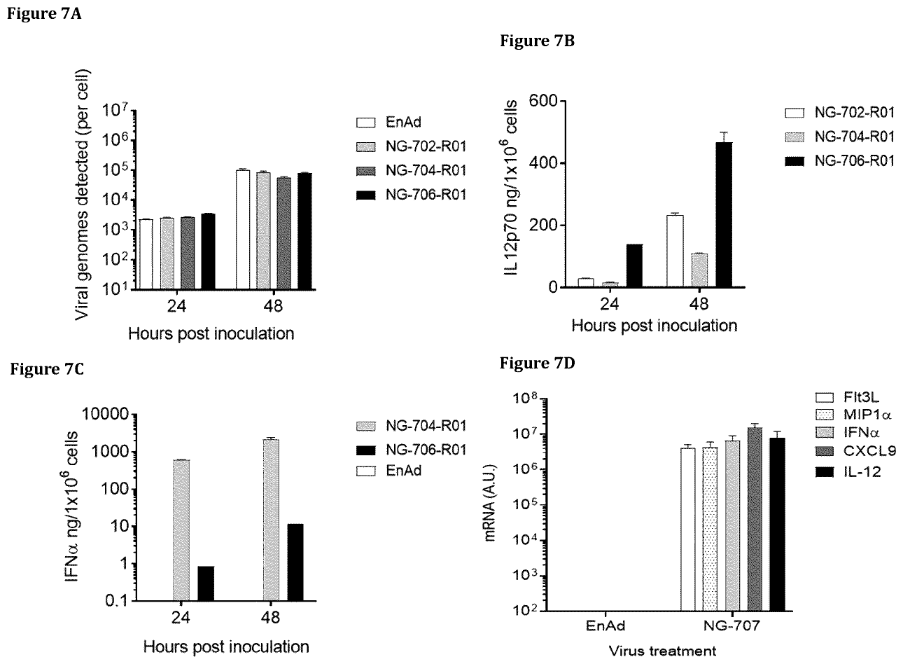

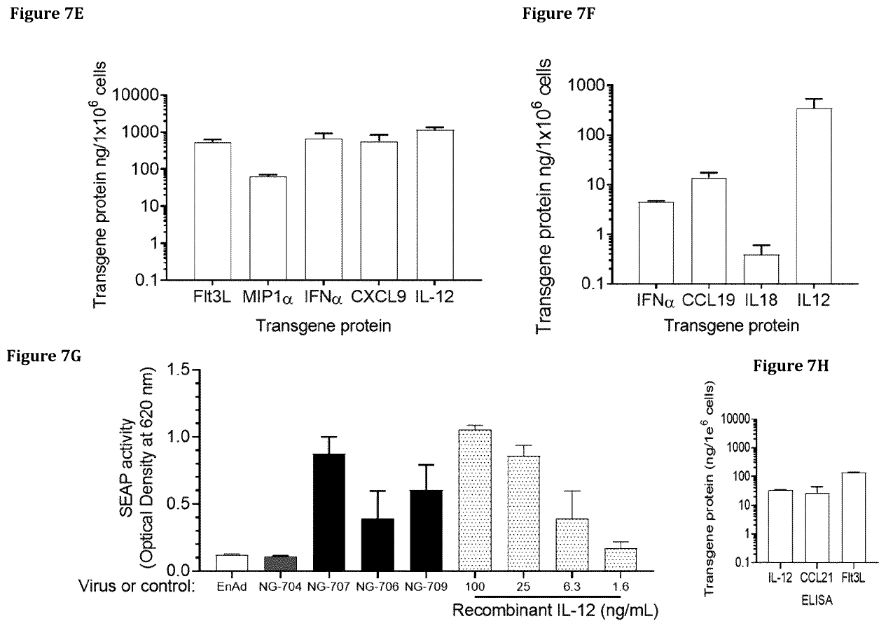

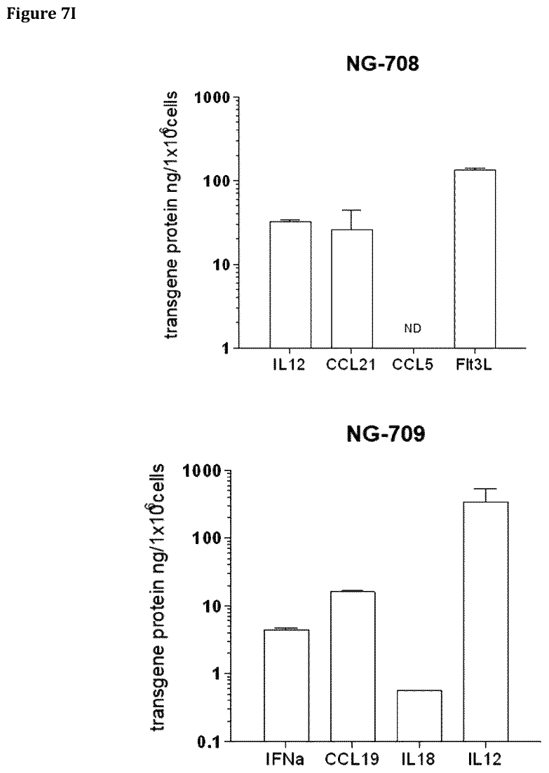

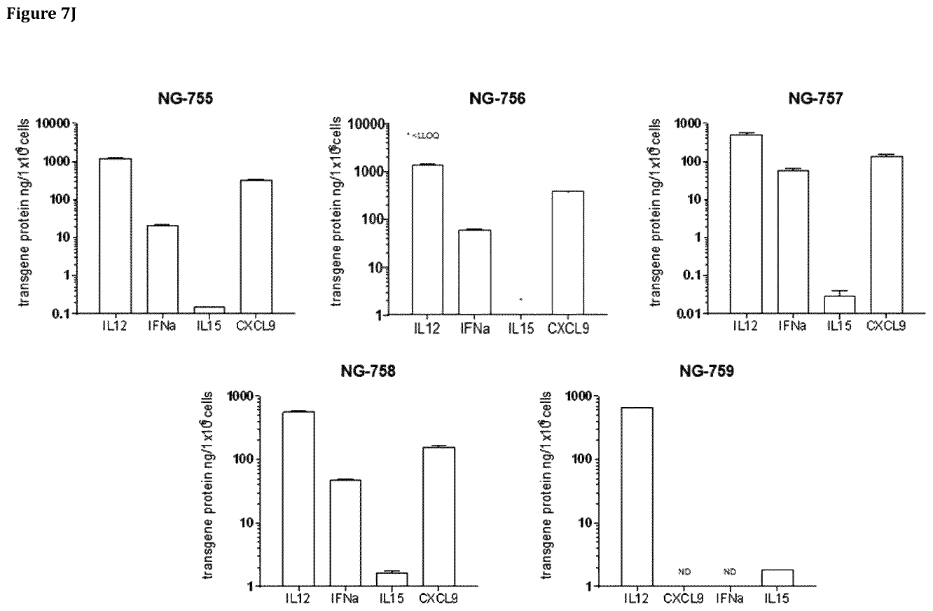

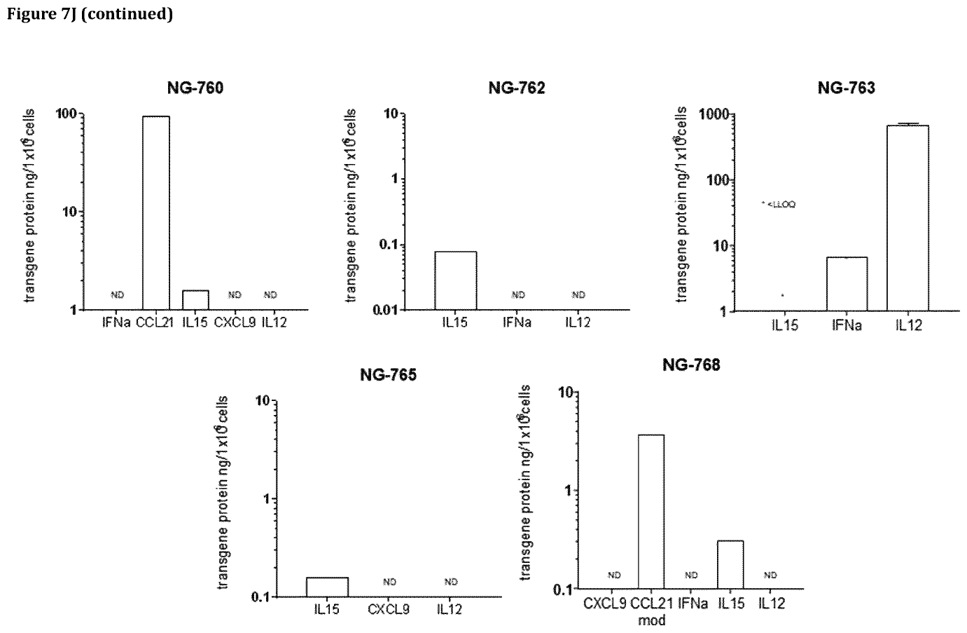

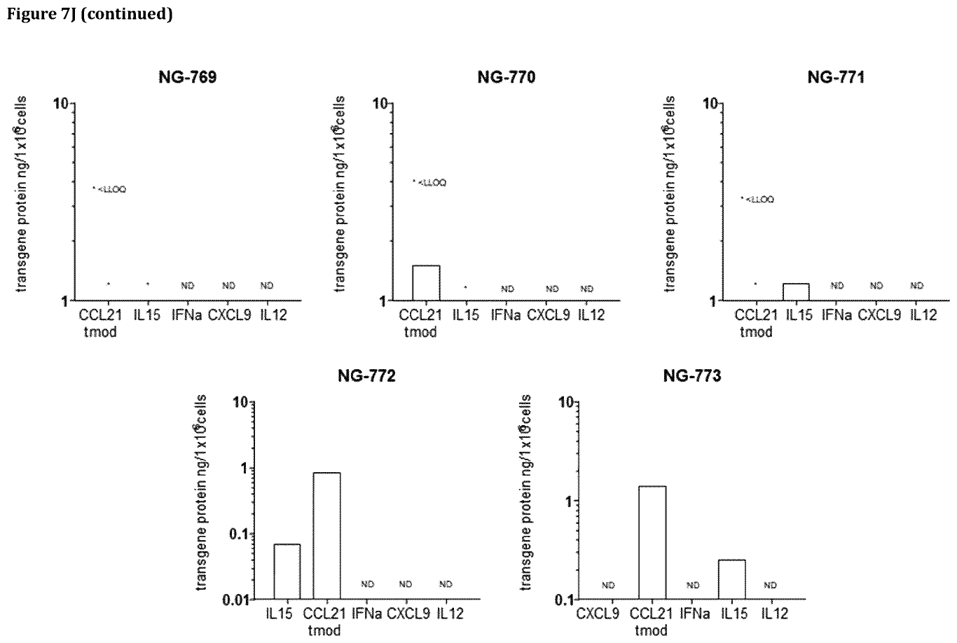

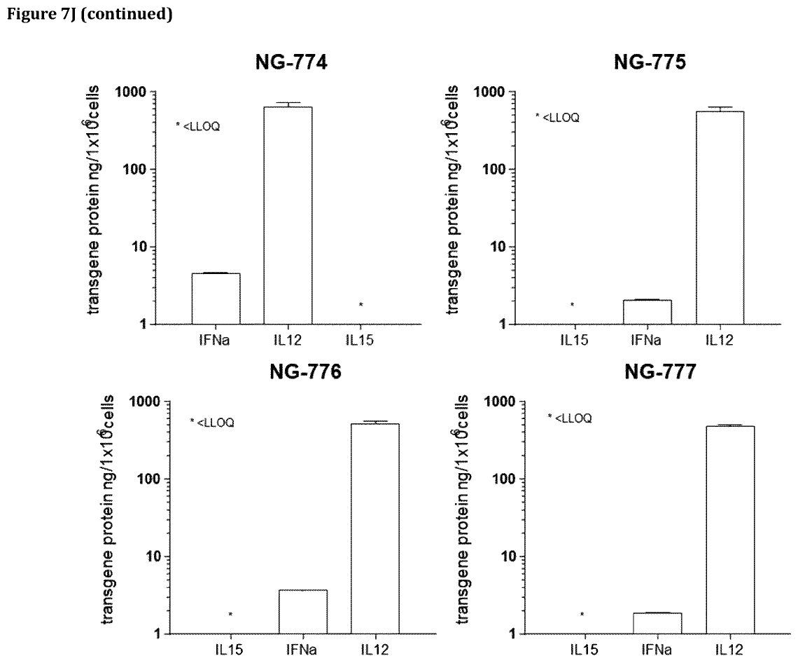

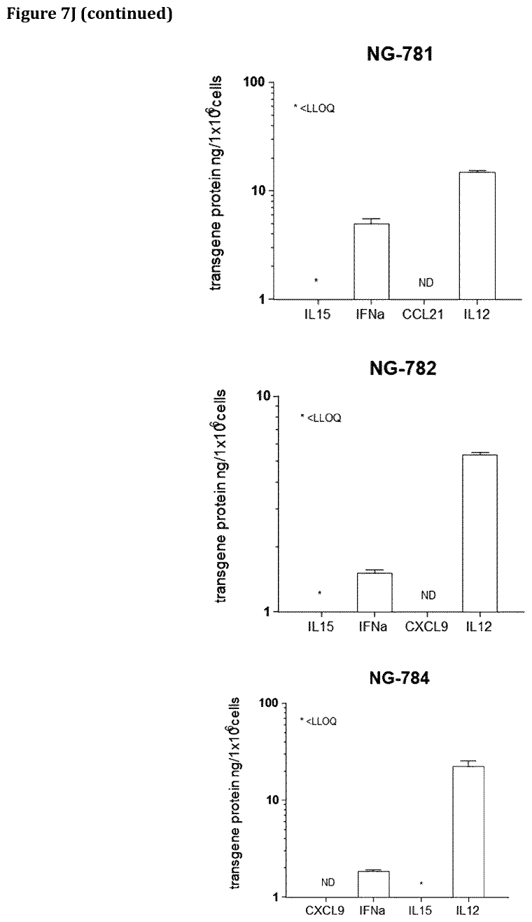

A549 cells were infected as described in Example 5, with either EnAd, NG-702, NG-704 or NG-706 at 100ppc. Supernatants and cells were harvested and clarified 24 or 48 hours later. Viral genomes were quantified for each time point by qPCR. Data demonstrated that each of the tested viruses produced similar quantities of viral genomes with similar kinetics (FIG. 7A). IL-12 p70 protein was quantified by ELISA from supernatant samples from the same experiment. ELISA data showed that all three viruses encoding single chain IL-12 produced IL-12 p70 protein, with NG-706 producing the most, and NG-704 the least (FIG. 7B). Quantification of IFNα˜ levels in the same supernatants by ELISA showed that NG-706 infected tumour cells produced less IFNα transgene protein than NG-704 infected cells (FIG. 7C).

In a second experiment, A549 human lung adenocarcinoma cells were seeded in 96 well plates at a cell density of 5×10⁴ cells/well and infected with either EnAd or NG-707 at 1ppc. Plates were incubated at 37° C., 5% CO2 before harvesting cells and supernatants 4 days later. Cells and supernatant samples were clarified and lysed as described previously. The cell fraction was analysed by RT-qPCR using primers targeting each of the encoded transgenes and data demonstrated that NG-707, but not EnAd, produced similar quantities of mRNA encoding each of the 5 encoded genes (FIG. 7D). IL-12 p70, Flt3 ligand (Flt3L), MIP1a, IFNα and CXCL9 transgene protein production was quantified by ELISA using supernatant samples from the same experiment ELISA data showed that NG-707 produced each of the 5 encoded proteins (FIG. 7E).

In a third experiment, A549 lung carcinoma cells were seeded and infected with NG-709, and supernatants harvested as described above. IL-12 p70, IFNα, MIP1a, CCL19 and IL-18 transgene protein production was quantified by ELISA, which showed that NG-709 produced each of the 4 encoded proteins (FIG. 7F).

In a fourth experiment, A549 lung carcinoma cells were seeded and infected with EnAd, NG-704, NG-706, NG-707 and NG-709 at 1ppc before harvesting and clarifying supernatants 72 hours later as described above. HEK-Blue IL-12 cells were seeded at 5×10⁴ cells per well in 96 well plates before being stimulated with supernatants (each pre-diluted 10-fold), or recombinant IL-12 (InvivoGen) at concentrations ranging from 100 to 1.6 ng/mL. Assay plates were incubated at 37° C., 5% CO2 for 20-24 hours before being centrifuged at 300×g for 5 minutes and removing clarified supernatant of which 20 μL was then transferred into a separate 96 well plate along with 180 μL of Quanti-Blue reagent Assay plates were incubated for 1 hour at 37° C. before analysing plates on a SpectraMax i3x plate reader with absorbance set to 620 nm. Data showed that NG-704 produced less functional IL-12 than the other IL-12 encoding viruses (FIG. 7G).

In a fifth experiment, A549 lung carcinoma cells were seeded and infected with NG-708 at 1ppc, and supernatants were harvested and clarified 96h later as described above. IL-12 p70, CCL21 and Flt3L transgene protein production was quantified by ELISA, which showed that NG-708 produced each of these 3 encoded proteins (FIG. 7H).

In a sixth experiment, A549 cells were seeded and infected with NG-708 or NG-709 at 10ppc, and supernatants were harvested and clarified after 72 hours as described above. IL-12, Flt3L, CCL19, CCL21, CCL5, IFNα and IL-18 transgene protein production was quantified by ELISA, which showed that NG-709 produced each of the 4 encoded proteins and NG-708 produced IL-12, CCL21 and Flt3L but CCL5 was not detected (ND) in this experiment (FIG. 7I).

In further experiments, other viruses depicted in FIG. 6 were used to infect A549 cells at 1ppc, and supernatants were harvested and clarified 96h later as described above and transgene protein production was quantified by ELISA. Levels of the different transgene proteins varied between different viruses, but IL-15 levels were consistently low or undetectable (FIG. 7J). For some of the viruses, some of the transgene protein levels were not determined (ND), or were tested but found to be below the lower limit of quantitation (LLOQ) of the assay.

### EXAMPLE 8: Production of IL-12 p70 by Viruses in Primary Human Tumour Samples

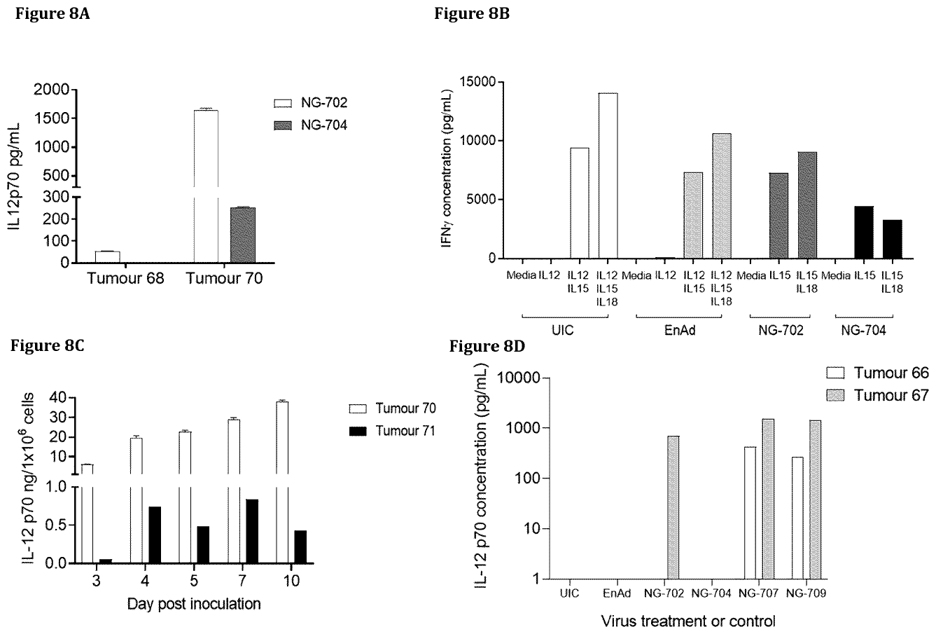

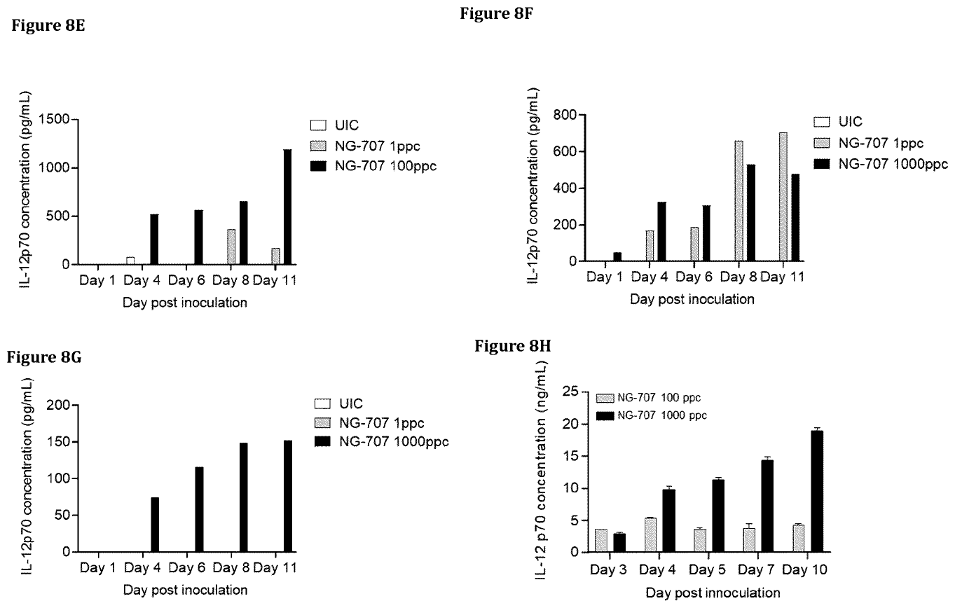

To evaluate virus and transgene protein activities using primary human tumour cells, surgically excised tumour samples were placed in Aqix organ transportation medium (supplemented with amphotericin B, penicillin, streptomycin, gentamycin and metronidazole) and shipped from the clinical site at 4° C. and obtained for processing in the laboratory within 24h. In two separate experiments, a colorectal tumour (CRC, tumour 68) and a kidney tumour (tumour 70) sample were cut into small pieces using scalpels and then enzymatically dissociated using a tumour dissociation mix (Miltenyi Biotech) on a Gentle MACS tissue disruptor (Miltenyi Biotech). Single cell suspensions were obtained by filtering and plated into 96 well or 24 well plates, depending on the cell yield in either RPMI (Gibco) supplemented with foetal bovine serum and insulin-transferrin or Cancer Cell Line Medium XF (PromoCell). Both media formulations were additionally supplemented with amphotericin B, penicillin and streptomycin. Cell types contained in these suspensions were routinely characterized by flow cytometry and shown to include tumour cells and different immune cell subsets, including T cells, B cells and NK cells. Single cell suspensions obtained from each tumour sample were either left uninfected, or infected with EnAd, NG-702 or NG-704 at 1000ppc. Recombinant proteins (IL-12 p70 at 15 ng/mL, IL-15 at 50 ng/mL, IL-18 at 50 ng/mL) were also added either alone, in combination or excluded to cover different permutations. Wells containing viruses expressing IL-12 were not supplemented with recombinant IL-12 p70. Supernatant samples were harvested and clarified as described in Example 7 at 48h (tumour 68) or 96h (tumour 70) post infection. IL-12 p70 protein was quantified by ELISA from supernatant samples from both experiments. ELISA data showed that both viruses encoding IL-12 produced IL-12 p70 protein from tumour 70, with NG-702 producing the most, while only NG-702 produced quantifiable levels from tumour 68 (FIG. 8A). Supernatant samples from tumour 70 cultures were also analysed by ELISA for the presence of IFNγ protein. The combination of IL-12+IL-15 stimulated the production of IFNγ both with recombinant IL-12, and where IL-12 was produced by cultures infected with NG-702 or NG-704. IL-12 alone, recombinant or encoded by NG-702 or NG-704, did not stimulate the production of IFNγ. In some cases, the addition of recombinant IL-18 to these cultures boosted IFNγ production further (FIG. 8B). No IL-15 alone control was included in this experiment, however the study summarised in FIG. 1A demonstrated that IL-15 alone induces similar levels of IFNγ to IL-12 alone, which in this study induced low or unquantifiable levels. The lower production of IFNγ from NG-704 infected wells correlate with the lower level of IL-12 production as compared to NG-702 in this experiment (FIG. 8A).

In a second experiment, tumour 70 cells and those from an additional dissociated CRC tumour (tumour 71) were both cultured and infected with NG-707 at 1000ppc. Supernatant and cell samples were harvested 3, 4, 5, 7 and 10 days post infection. Cells and supernatant samples were clarified and lysed as described previously. IL-12 p70 protein was quantified by ELISA from supernatant samples. ELISA data showed that NG-707 produced IL-12 p70 protein from both tumour samples at all measured time points (FIG. 8C).

In a third experiment, two breast tumour samples (tumour 66 and 67) were dissociated as described previously, cultured and either left uninfected (UIC), or infected with EnAd, NG-702, NG-704, NG-707 or NG-709 at 1000ppc. Supernatant and cell samples were harvested 4 days post infection. Supernatant samples were clarified as described previously. IL-12 p70 protein was quantified by ELISA from supernatant samples. ELISA data showed that NG-707 and NG-709 produced detectable IL-12 p70 protein from both tumour samples, while NG-702 only produced detectable protein from tumour 67, and NG-704 failed to produce detectable protein from either tumour sample (FIG. 8D).

In a fourth experiment, a colorectal tumour sample (tumour 75) was dissociated and cultured as described earlier and either left uninfected (UIC) or infected with NG-707 at 1ppc or 100ppc. Supernatant samples were harvested 1, 4, 6, 8 and 11 days post inoculation, clarified as described previously and IL-12 p70 protein quantified by ELISA. ELISA data showed that NG-707 produced detectable IL-12 p70 protein from day 4 at 100ppc, and from day 8 with 1ppc (FIG. 8E). Note: IL-12 protein detected at day 4 in the uninfected control is likely due to low level production by immune cells present in the heterogeneous mix of cells present in the tumour samples.

In a fifth experiment, a colorectal tumour sample (tumour 76) was dissociated and cultured as described earlier and either left uninfected (UIC) or infected with NG-707 at 1ppc or 1000ppc. Supernatant samples were harvested 1, 4, 6, 8 and 11 days post inoculation, clarified as described previously and IL-12 p70 protein quantified by ELISA. ELISA data showed that NG-707 produced detectable IL-12 p70 protein from day 1 at 1000ppc, and from day 4 with 1ppc (FIG. 8F).

In a sixth experiment, a colorectal tumour sample (tumour 79) was dissociated and cultured as described earlier and either left uninfected (UIC) or infected with NG-707 at 1ppc or 1000ppc. Supernatant samples were harvested 1, 4, 6, 8- and 11-days post inoculation, clarified as described previously and IL-12 p70 protein quantified by ELISA. ELISA data showed that NG-707 produced detectable IL-12 p70 protein from day 4 at 1000ppc, with 1ppc not leading to the production of detectable IL-12 at any time point (FIG. 8G).

In a seventh experiment, a primary renal cell carcinoma sample (tumour 70) was dissociated as described earlier and either left uninfected (UIC) or infected with NG-707 at 100ppc or 1000ppc. Supernatant samples were harvested 3, 4, 5, 7- and 10-days post inoculation, clarified as described previously and IL-12 p70 protein quantified by ELISA. Data showed that NG-707 produced detectable IL-12 p70 protein at higher levels with 1000ppc than with 100ppc (FIG. 8H).

### EXAMPLE 9: Activity of Viruses Encoding Human IL-15 Together with the IL-15 Binding Region of the Human IL-15 Receptor Alpha (IL-15Ra) in the Transgene Cassette

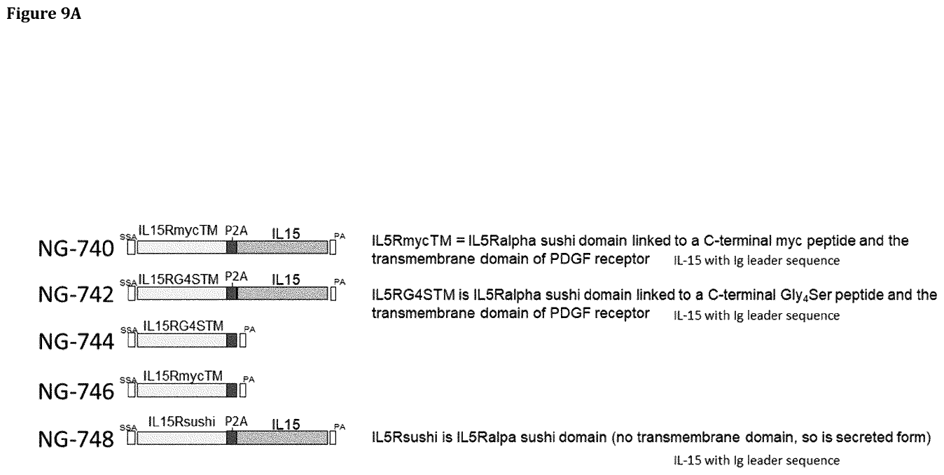

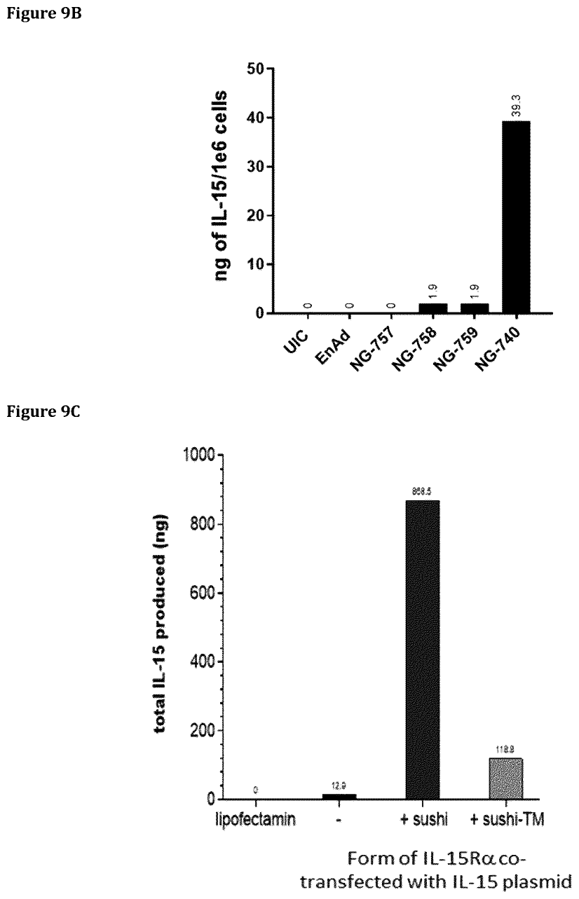

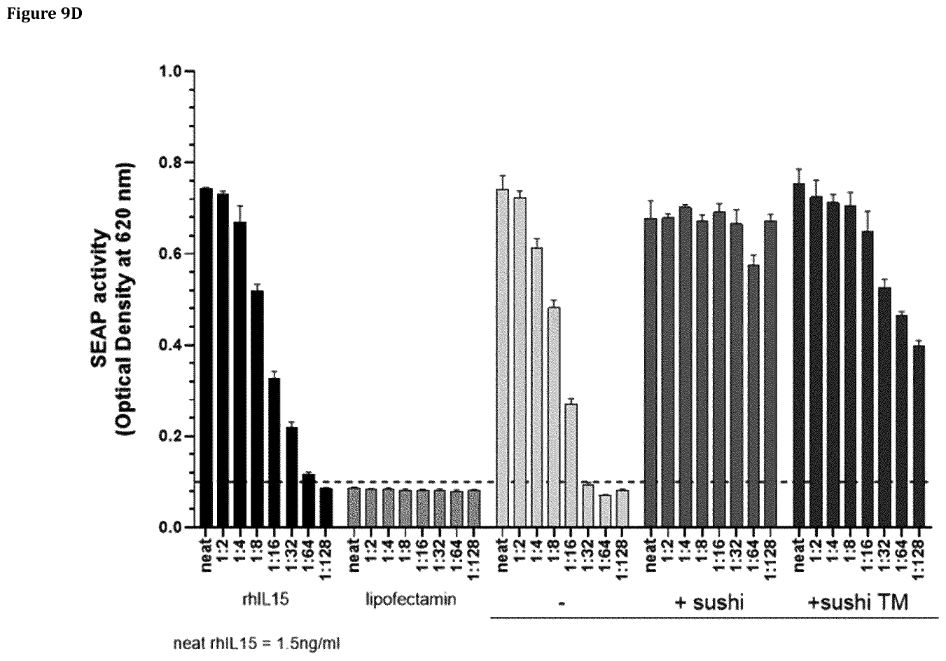

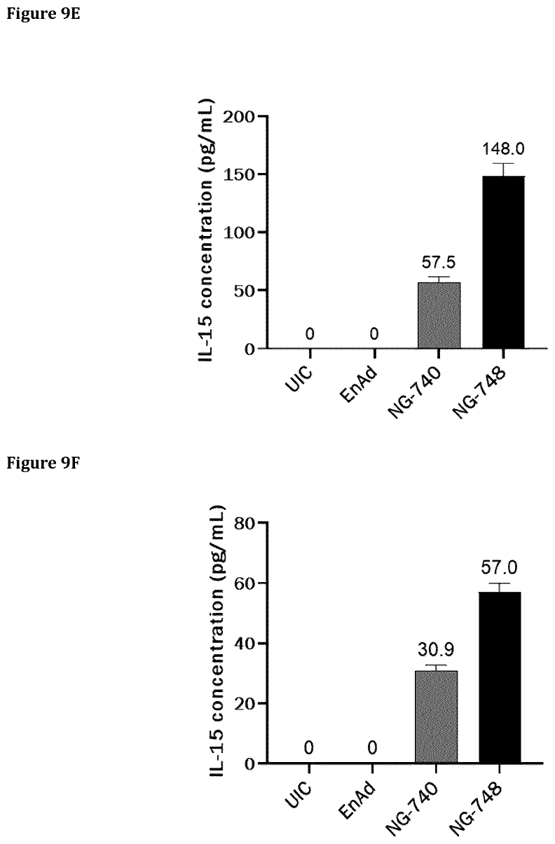

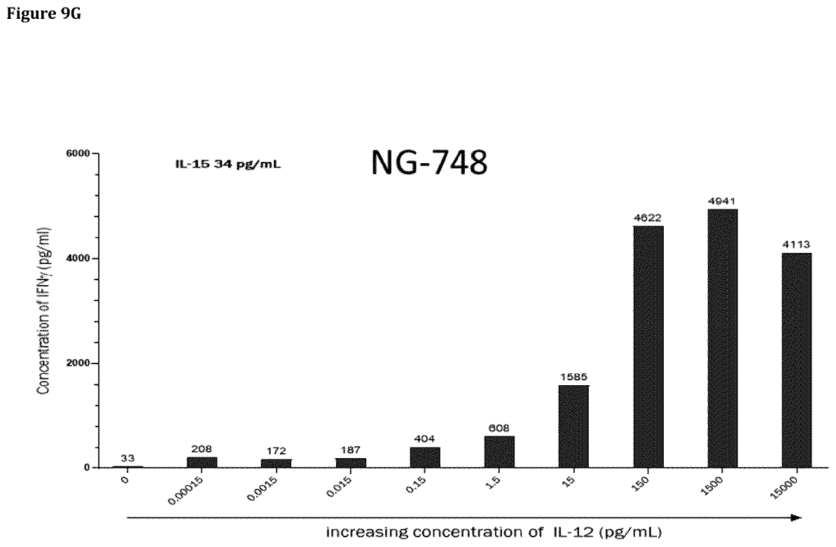

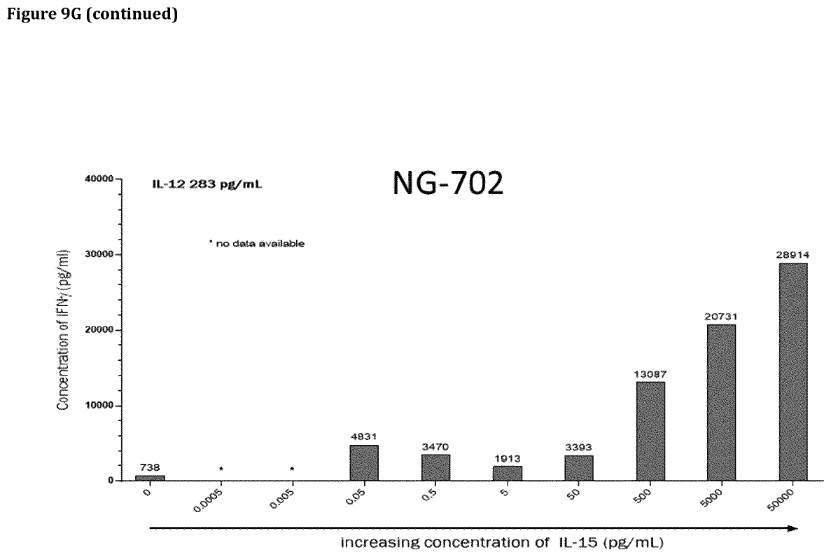

Functional signalling by IL-15 involves it binding first to IL-15Ra molecules which then together bind to cells bearing receptors comprising the common gamma chain (gc) and IL-2 receptor beta (IL-2Rb). To investigate the effect of encoding an IL-15 binding domain on the functional activity of an IL-15 transgene, a membrane-anchored form of the main IL-15 binding region of the IL-15Ra (the “Sushi” domain) was created by linking the sequence to the transmembrane region of the PDGF receptor via either a cMyc peptide or a Gly4Ser linker. These transgene sequences were used to create viruses NG-744 and NG-746, as well as NG-740 and NG-742 which also encode IL-15 (Table 3, FIG. 9A).

TABLE 3

| | |
|---|---|
| NG-740 | SSA¹-IL15RsushimycTM²⁷- |
| (SEQ ID NO. 89) | P2A³-IL15¹⁹-PA⁵ |
| | (SEQ ID NO. 48) |
| NG-742 | SSA¹-IL15RsushiG4STM²⁸- |
| (SEQ ID NO. 90) | P2A³-IL15¹⁹-PA⁵ |
| | (SEQ ID NO. 49) |
| NG-744 | SSA¹-IL15RsushiG4STM²⁸- |
| (SEQ ID NO. 91) | PA⁵ (SEQ ID NO. 50) |
| NG-746 | SSA¹-IL15RsushimycTM²⁷- |
| (SEQ ID NO. 92) | PA⁵ (SEQ ID NO. 51) |
| NG-748 | SSA¹-IL15Rsushi²⁹-P2A³- |
| (SEQ ID NO. 211) | IL15¹⁹-PA⁵ |
| | (SEQ ID NO. 167) |

¹SEQ ID NO. 1; ³SEQ ID NO. 4; ⁵SEQ ID NO. 8; ¹⁹SEQ ID NO. 21; ²⁷SEQ ID NO. 25; ²⁸SEQ ID NO. 29; ²⁹SEQ ID NO. 26

Viruses were produced and purified using the protocol described in Example 6.

In a first experiment, NG-740 inoculation of A549 cells led to increased IL-15 production by ELISA compared to other viruses (NG-757, NG-758, NG-759) encoding an IL-15 transgene but no IL-15Ra (FIG. 9B).

To evaluate the effect on IL_15 production of expressing either transmembrane-anchored or soluble secreted forms of IL-15Ra sushi domain, a transfection approach was first undertaken using pUC vectors expressing gene products under control of a CMV promoter. Transfection of HEK-293 cells with an IL-15 pUC vector alone or together with a separate vector expressing either the soluble secreted or transmembrane anchored forms of IL-15Ra sushi domain showed that either form of IL-15Ra increased production of IL-15 as measured by ELISA (FIG. 9C) or using a functional reporter assay (FIG. 9D). This reporter assay used HEK-BlueIL-2 cells (InVivogen) which, because HEK293 cells constitutively express native IL-15Ra on their cell surface, respond to IL-15 as well as IL-2 to produce the secreted alkaline phosphatase reporter protein that is measured using a colorimetric enzyme assay.

A further virus, NG-748, was then constructed and prepared (as in Example 6) that produced a soluble (secreted) version of the IL-15Ra sushi domain together with IL-15 (Table 3, FIG. 9A). Inoculation of A549 cells (FIG. 9E) or a primary colorectal tumour cell sample (FIG. 9F) with either NG-740 or NG-748 showed that both led to production of IL-15, as measured by ELISA, with higher levels produced with NG-748 which encodes the soluble secreted IL-15Ra sushi domain.

In another study, primary kidney tumour cells (tumour 101) were treated with NG-748 or NG-702, in the presence of different levels of added recombinant IL-12 or IL-15, respectively. Production of IFNg by the primary TILs in the cultures was measured by ELISA of culture supernatants. The data (FIG. 9G) show a strong, dose-dependent synergy of IFNg induction by the IL-15 producing NG-748 virus with added IL-12, and a similar dose-dependent synergy of the IL-12 producing virus NG-702 with added IL-15.

### EXAMPLE 10: Viruses Encoding IL-15Rsushi Designed to Secrete Both IL-12 and IL-15

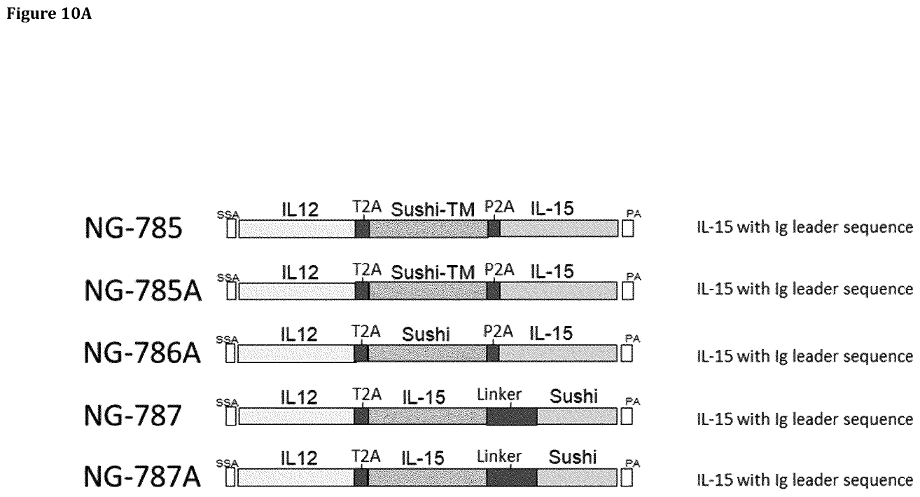

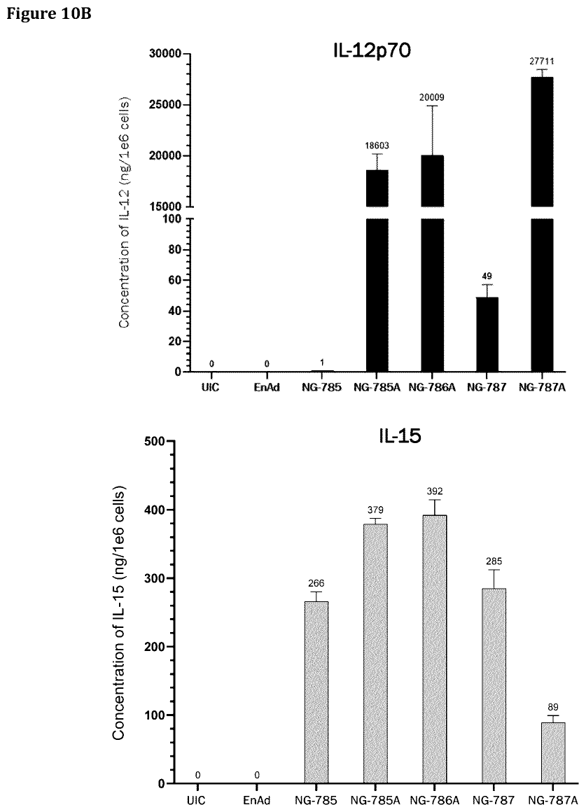

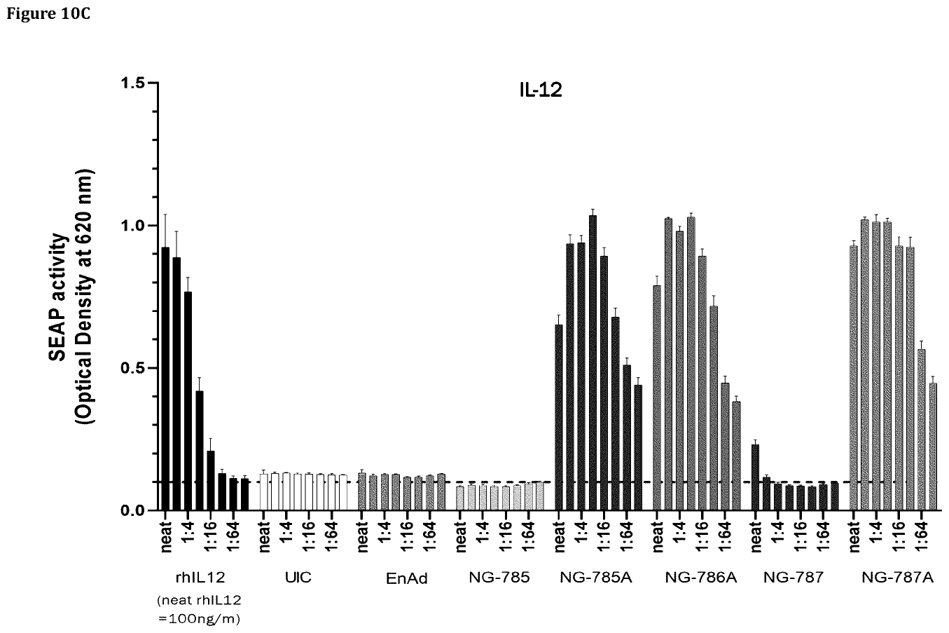

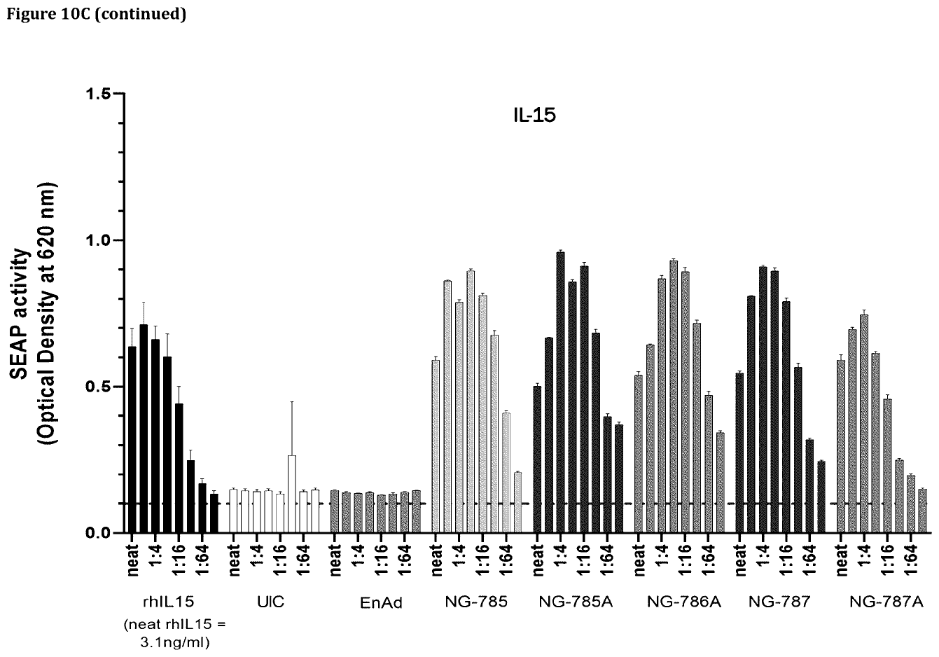

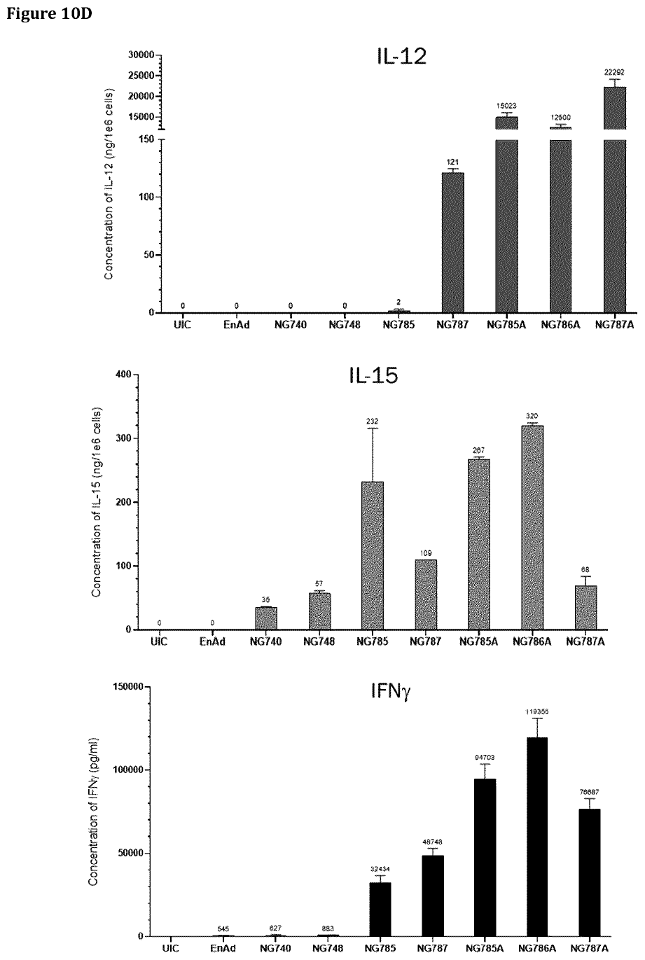

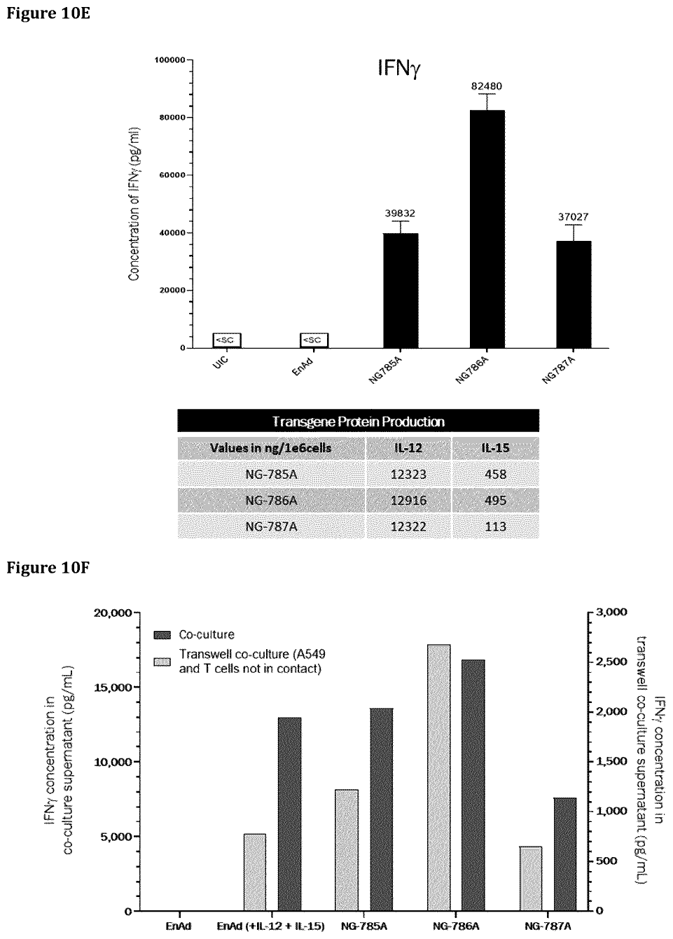

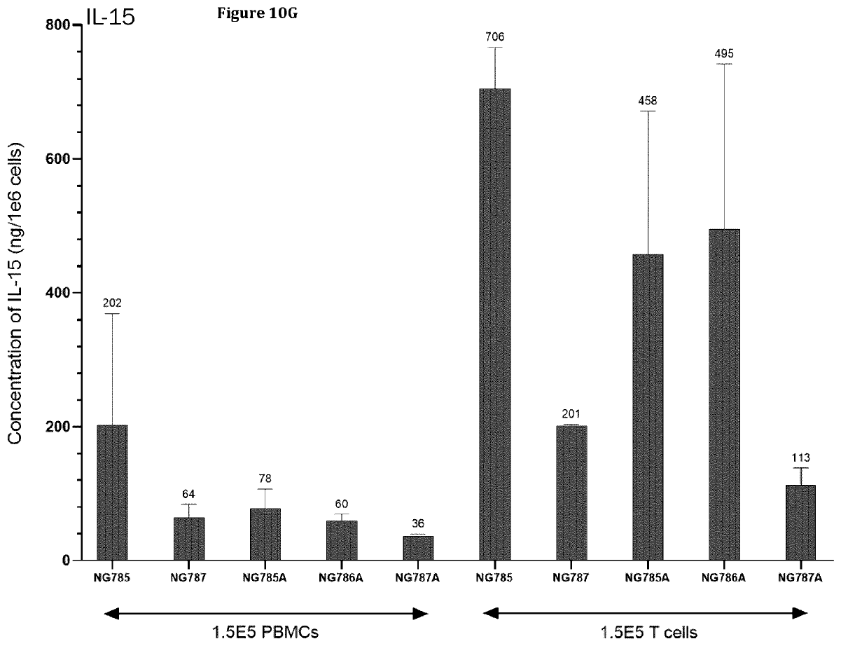

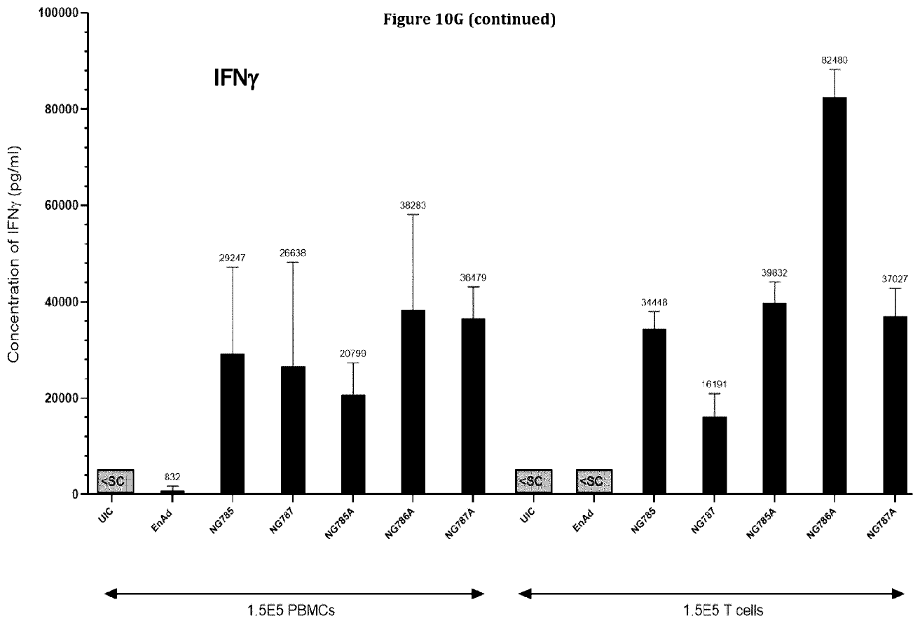

Further viruses were designed to incorporated different forms of the IL-15Ra sushi domain together with both IL-12 and IL-15 transgenes. Viruses NG-785, NG-785A, NG-786A, NG-787 and NG-787A (Table 4; FIG. 10A) were produced and purified as described in Example 6.

TABLE 4

| | |
|---|---|
| NG-785 | SSA¹-IL12p40LinkerIL12p35⁶- |
| (SEQ ID NO. 219) | T2A¹¹-IL15RsushimycTM²⁷- |
| | P2A³-IL15¹⁹-PA⁵ (SEQ ID NO. 175) |
| NG-785A | SSA¹-IL12p40LinkerIL12p35⁶- |
| (SEQ ID NO. 20) | T2A¹¹-IL15RsushimycTM²⁷- |
| | P2A³-IL15¹⁹-PA⁵ (SEQ ID NO.176) |
| NG-786A | SSA¹-IL-12p40LinkerIL12p35⁶- |
| (SEQ ID NO. 221) | T2A¹¹-IL15Rsushi²⁹-P2A³- |
| | IL15¹⁹-PA⁵ (SEQ ID NO. 177) |
| NG-787 | SSA¹-IL12p40LinkerIL12p35⁶- |
| (SEQ ID NO. 222) | T2A¹¹-IL15Rsushi-Linker- |
| | IL15³⁰-PA⁵ (SEQ ID NO. 178) |
| NG-787A | SSA¹-IL12p40LinkerIL12p35⁶- |
| (SEQ ID NO 223) | T2A¹¹-IL15Rsushi-Linker- |
| | IL15³⁰-PA⁵ (SEQ ID NO. 179) |

¹SEQ ID NO. 1; ³SEQ ID NO. 4; ⁵SEQ ID NO. 8; ⁶SEQ ID NO. 11; ¹¹SEQ ID NO. 5; ¹⁹SEQ ID NO. 21; ²⁷SEQ ID NO. 25; ²⁹SEQ ID NO. 26; ³⁰SEQ ID NO. 236

To assess transgene protein production by these viruses, A549 cells were infected with 1ppc of viruses and supernatants collected after 96h for testing in ELISA and functional reporter assays for IL-12 and IL-15. ELISA data showed good expression levels of IL-12 and IL-15 from all five viruses, with the exception of a very low level of IL-12 being produced by NG-785 (FIG. 10B). Testing different dilutions of these same supernatants in IL-12 and IL-15 functional reporter assays (as described in Example 5 and Example 9) generated data that aligned with the ELISA results, demonstrating functionality of the transgene proteins made (FIG. 10C).

In a second study, A549 cells left untreated or inoculated with 1ppc of EnAd, NG-740, NG-748 or the viruses listed in Table 4 and PBMCs added after 24h to assess the ability of cytokines produced by the viruses to stimulate immune cell responses. Supernatants were then collected after 72h and assessed by ELISA for levels of IL-12 and IL-15 transgene protein as well as IFNg produced by the added immune cells. The data showed that the five viruses expressing both IL-12 and IL-15 with IL-15Rsushi variants induced much higher IFNg production than NG-740 and NG-748 which only express IL-15 with IL-15Rsushi variants not IL-12 (FIG. 10D).

A further experiment, A549 cells were inoculated with 1ppc of NG-785A, NG-786A or NG-787A or controls and after 24 hours culture, human CD3+ T-cells (purified from PBMCs) were added and at 72 hours culture supernatants were assessed for IFNg levels by ELISA as a functional assessment of the transgene protein production by the viruses. All three transgene bearing viruses produced their encoded IL-12 and IL-15 proteins leading to the production of high levels of IFNg, indicating activation of the added T-cells, whereas the control virus EnAd did not (FIG. 10E).

A further experiment with these same three viruses compared culture conditions where the virus-inoculated A549 tumour cells were in contact with the added responder T-cells with culturing in a transwell plate where the T-cells are separated from the virus inoculated tumour cells to remove direct contact between them. This enabled a comparison of the T-cell activation achieved when the IL-15Rsushi transgene protein can move across the transwell filter along with the IL-12 and IL-15 transgene proteins (NG-786A) with the IL-15Rsushimyc™ which cannot (NG-785A) as it stays in the membrane of the incoculated tumour cells. EnAd or EnAd with recombinant IL-12 and IL-15 added were used as comparator controls. Viruses were used at 1ppc and T-cells added to the two culture types 24h later. IFNg in the T-cell culture supernatants measured by ELISA shows that when the T-cells are separated from the transgene protein producing tumour cells, using a secreted version of the IL-15Ra sushi domain (NG-786A) led to higher levels of activation compared to the transmembrane IL-15Ra sushi domain form (NG-785A) and also higher than achieved without an IL-15Ra transgene as shown using EnAd inoculation with recombinant IL-12 and IL-15 (FIG. 10F).

In a similar study to that shown in FIG. 10D, either PBMCs or purified T-cells were added to assess IL-12/IL-15 mediated immune cell activation. The data again showed that all five transgene-bearing viruses tested led to activation of IFNg production (FIG. 10G).

### EXAMPLE 11: Evaluation of Activity of Viruses IL-12, IL-15 and IL-5Rsushi in Primary Human Tumour Cell Cultures

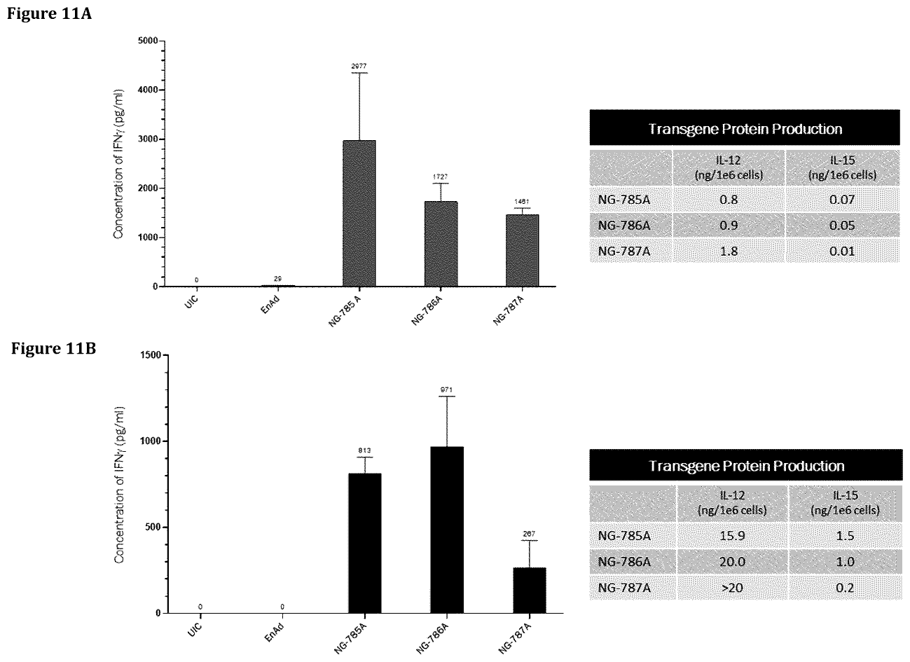

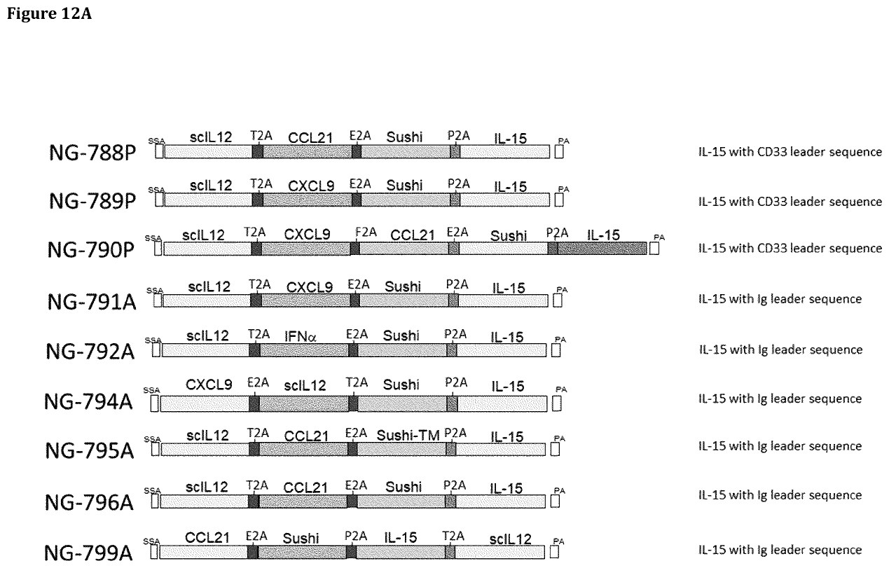

Primary human tumour samples from two colorectal cancer patients were processed and cultured as described in Example 1. Replicate cultures were inoculated with 1000ppc of EnAd, NG-785A, NG-786A or NG-787A, or left untreated. After 72h (Tumour 98, Figure A) or 96h (Tumour 99, FIG. 11B), supernatants were removed and levels IFNg were measured by ELISA as a measure of activation of the endogenous tumour infiltrating lymphocytes in the tumour samples. As shown in FIG. 11, all the transgene-expressing viruses led to production of IL-12 and IL-15 by the primary tumour cells and all led to stimulation of IFNg production.

### EXAMPLE 12: Generation and Characterization of IL-5Rsushi, IL-12 and IL-15 Viruses Additionally Expressing a Fourth Transgene

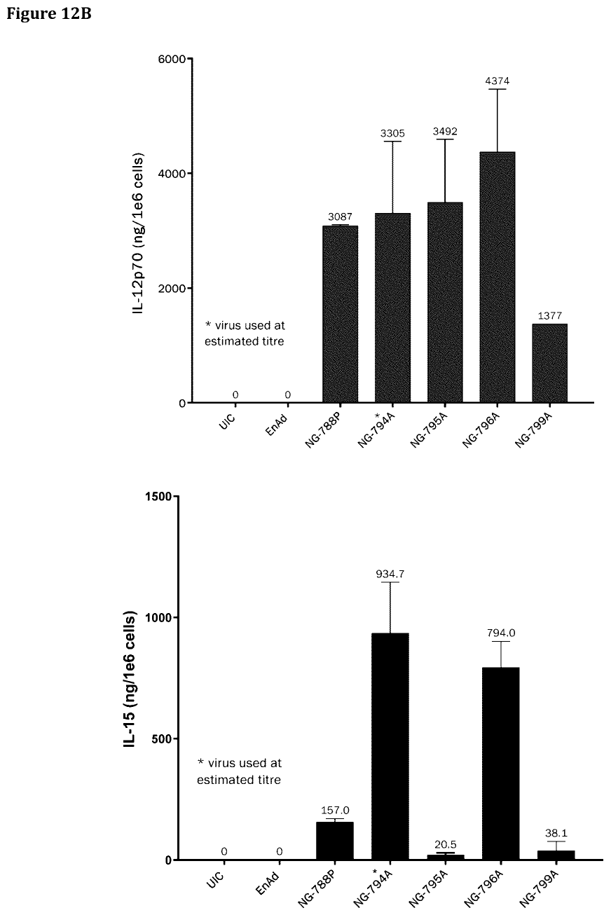

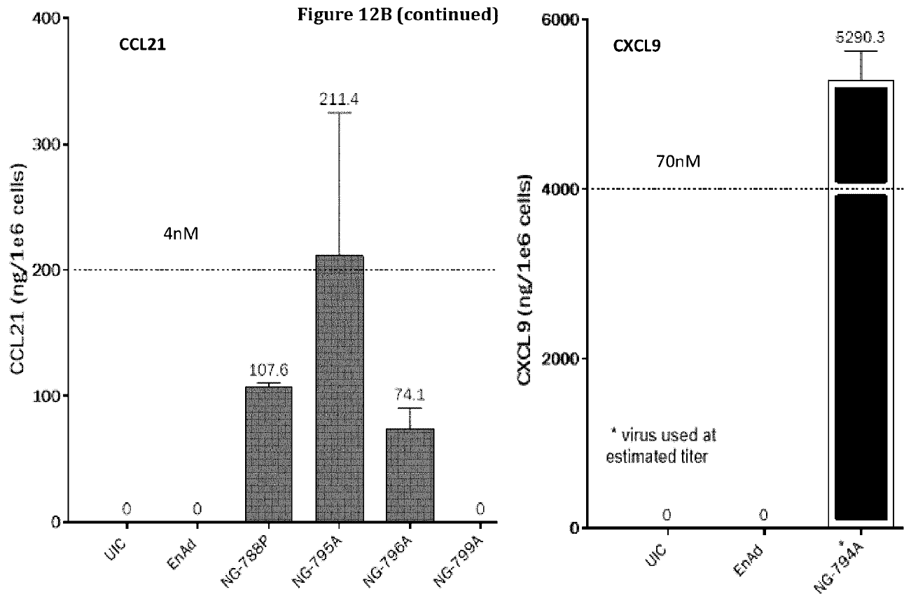

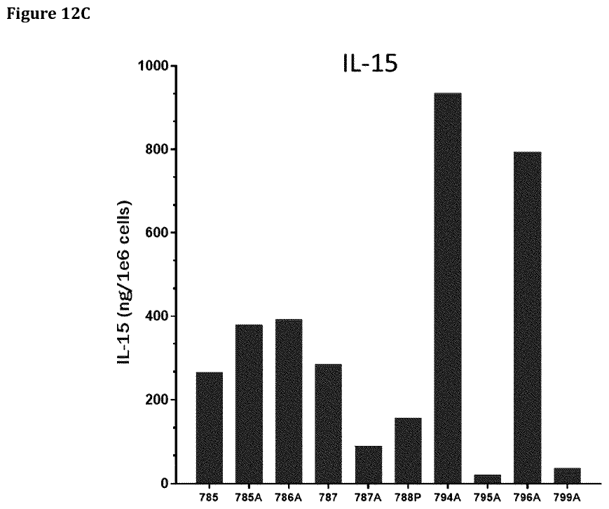

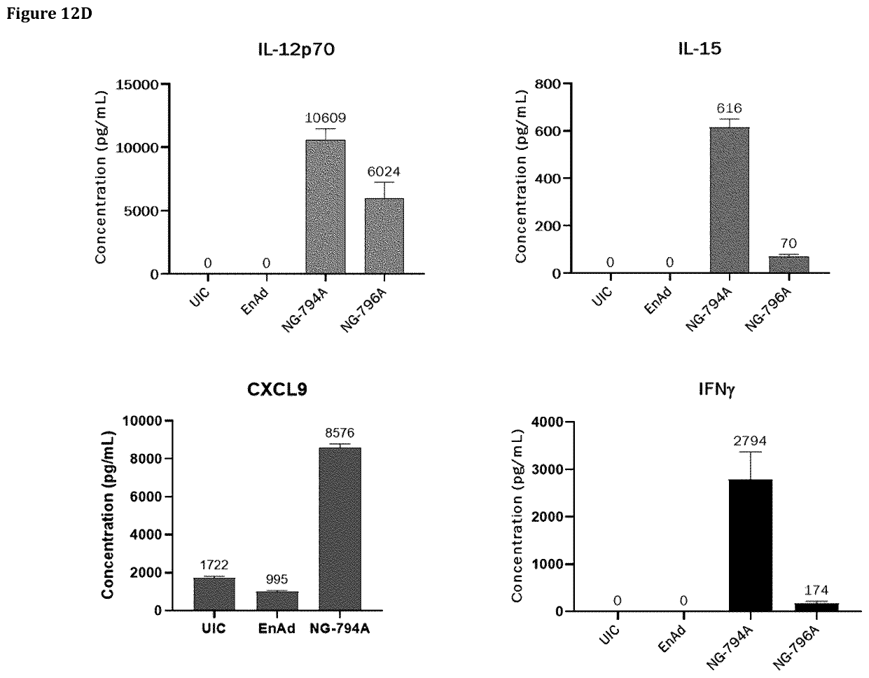

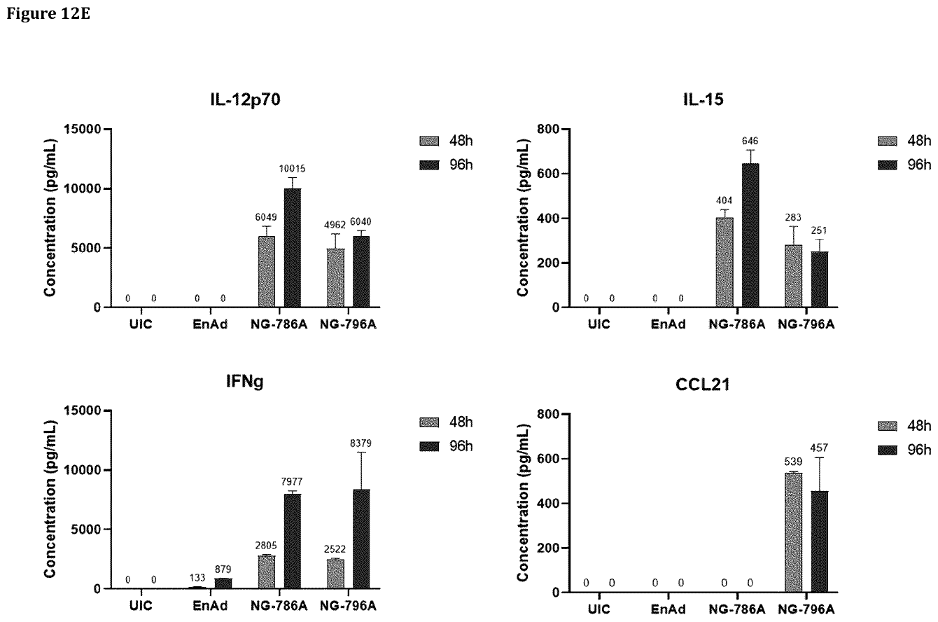

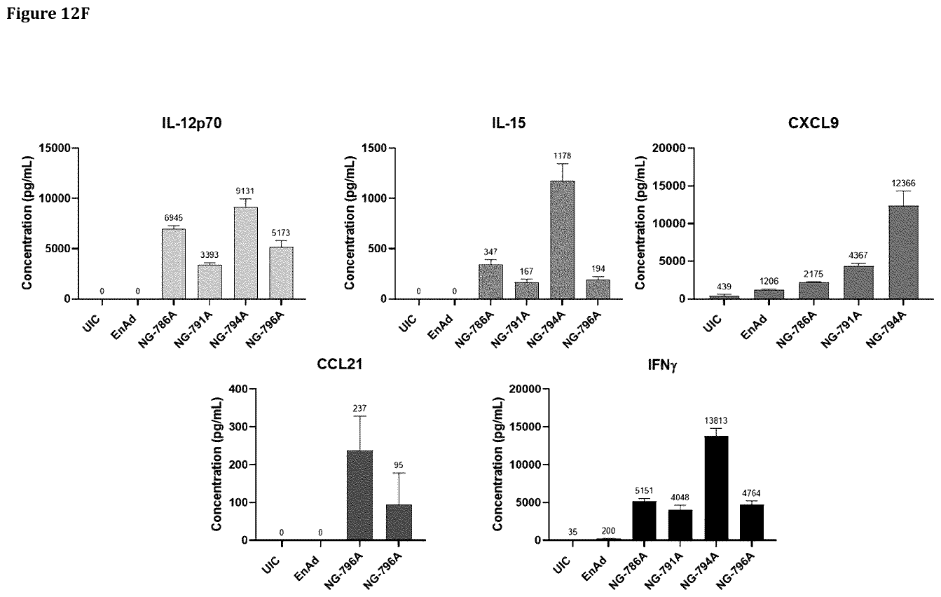

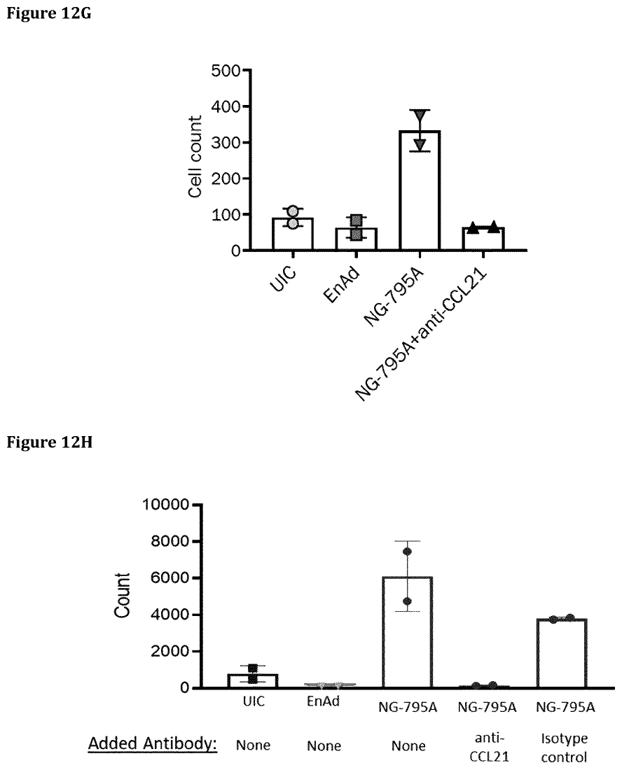

A further set of viruses having IFNα, CXCL9 or CCL21 as an additional one or two transgenes encoded in the transgene cassette along with IL-5Rsushi, IL-12 and IL-15 were designed produced as described in Example 6 (Table 5; FIG. 12A).

TABLE 5

| | |
|---|---|
| NG-788P | SSA¹-IL12p40LinkerIL12p35⁶-T2A¹¹- |
| (SEQ ID NO. 224) | CCL21²⁸-E2A¹²-IL15Rsushi²⁹-P2A³- |
| | IL15²⁵-PA⁵ (SEQ ID NO. 180) |
| NG-789P | SSA¹-IL12p40LinkerIL12p35⁶-T2A¹¹- |
| (SEQ ID NO. 225) | CXCL9¹³-E2A¹²-IL15Rsushi²⁹-P2A³- |
| | IL15²⁵-PA⁵ (SEQ ID NO. 181) |
| NG-790P | SSA¹-IL12p40LinkerIL12p35⁶-T2A¹¹ |
| (SEQ ID NO. 226) | -CXCL9¹³-F2A¹⁴-CCL21²⁸-E2A¹²- |
| | IL15Rsushi²⁹-P2A³-IL15²⁵-PA⁵ |
| | (SEQ ID NO. 182) |
| NG-791A | SSA¹-IL12p40LinkerIL12p35⁶-T2A¹¹- |
| (SEQ ID NO. 227) | CXCL9¹³-E2A¹²-IL15Rsushi²⁹-P2A³- |
| | IL15¹⁹-PA⁵ (SEQ ID NO. 183) |
| NG-792A | SSA¹-IL12p40LinkerIL12p35⁶-T2A¹¹- |
| (SEQ ID NO. 228) | IFNa⁸-E2A¹²-IL15Rsushi²⁹-P2A3_ |
| | IL15¹⁹-PA⁵ (SEQ ID NO. 184) |
| NG-794A | SSA¹-CXCL9¹³-E2A¹²-IL12p40Linker |
| (SEQ ID NO. 229) | IL12p35⁶-T2A¹¹-IL15Rsushi²⁹-P2A3_ |
| | IL15¹⁹-PA⁵ (SEQ ID NO. 185) |
| NG-795A | SSA¹-IL12p40LinkerIL12p35⁶-T2A¹¹- |
| (SEQ ID NO. 230) | CCL21²⁸-E2A¹²- |
| | IL15RsushimycTM²⁷-P2A³-IL15¹⁹-PA⁵ |
| | (SEQ ID NO. 186) |
| NG-796A | SSA¹-IL12p40LinkerIL12p35⁶-T2A¹¹- |
| (SEQ ID NO. 231) | CCL21²⁸-E2A¹²-IL15Rsushi²⁹-P2A³- |
| | IL15¹⁹-PA⁵ (SEQ ID NO. 187) |
| NG-799A | SSA¹-CCL21²⁸-E2A¹²-IL15Rsushi²⁹- |
| (SEQ ID NO 232) | P2A³- IL15¹⁹-T2A¹¹- |
| | IL12p40LinkerIL12p35⁶-PA⁵ |
| | (SEQ ID NO. 188) |

¹SEQ ID NO. 1; ³SEQ ID NO. 4; ⁵SEQ ID NO. 8; ⁶SEQ ID NO. 11; ⁸SEQ ID NO. 13; ¹¹SEQ ID NO. 5; 12 SEQ ID NO. 6; ¹³SEQ ID NO. 16; ¹⁴SEQ ID NO. 7; ¹⁹SEQ ID NO. 21; ²⁵SEQ ID NO. 163; ²⁷SEQ ID NO. 25; ²⁸SEQ ID NO. 17; ²⁹SEQ ID NO. 26

Viruses NG-788P, NG-794A, NG-795A, NG-796A and NG-799A were characterized for production of their transgene proteins, by specific ELISA assays, by infecting A549 cells, with uninfected (UIC) or EnAd infected A549 cells serving as controls. With the exception of NG-799A, all viruses made detectable levels of all their encoded transgene proteins, with IL-15 levels being notably higher with NG-794A and NG-796A (FIG. 12B). NG-799A did not make detectable levels of CCL21 and the level of IL-15 was also low.

In a similar second study, the same 5 viruses were tested alongside the viruses listed in Table 4, Example 10 to compare the levels of IL-15 transgene protein produced by viruses co-expressing IL-12 and IL-15 with IL-15Rsushi. The IL-15 ELISA data from the A549 cell culture supernatants (FIG. 12C) shows detectable IL-15 from all the viruses, with the highest levels from using NG-794A and NG-796A.

NG-794A and NG-796A were then tested for their effects on primary human tumour cell cultures established as described in Example 1 using a colorectal tumour sample (tumour 105) inoculated with viruses at 1000ppc and supernatants collected for cytokine analyses by ELISA after 48h. The data (FIG. 12D) show that both transgene-bearing viruses produced their respective transgenes and led to the activation of IFNg production.

In a similar study, a different colorectal tumour sample (tumour 107) was treated with NG-786A or NG-796A and compared to EnAd or no treatment. As shown in FIG. 12E, both transgene-bearing viruses produced their transgene proteins and led to activation of IFNg production by these primary tumour cells.

In a further study, another colorectal tumour sample (tumour 112) was treated with NG-786A, NG-791A, NG-794A or NG-796A and compared to EnAd or no treatment. As shown in FIG. 12F, all transgene-bearing viruses produced their transgene proteins and led to activation of IFNg production by these primary tumour cells.

The production of IFNg by PBMC-derived T-cells or primary tumour cell cultures served to test the functionality of the IL12 and IL-15/IL-15Rsushi transgene proteins. To demonstrate functionality of the CCL21 transgene product, monocyte-derived dendritic cells prepared from PBMCs (by culturing them with GM-CSF and IL-4 as described in Example 3) were stimulated with LPS for 24 before using in a transwell migration assay. The assay was run using an 8 mm pore-size transwell coated with 500 mg/mL Matrigel, as described in Example 3. Culture supernatants from uninfected A549 cells or A549 cells treated with EnAd or NG-795A were placed in the bottom well, with or without a blocking antibody to CCL21 (pre-incubated for 1 hour at room temperature) and dendritic cells added to the top wells. After 12h of culture, the numbers of dendritic cells that had migrated into the lower chamber were measured by flow cytometry. The data in FIG. 12G show that dendritic cell migration was increased selectively by the CCL21-containing supernatant from NG-795A and this was inhibited by the anti-CCL21 antibody, showing that the CCL21 transgene product is functional as a chemokine for dendritic cells. Similar data were also obtained from a repeat experiment where the dendritic cells were labelled with 1 mM Cell Tracer Violet (Invitrogen) after LPS stimulation and prior to use in the migration assay, and an isotype matched antibody was also included as an irrelevant antibody control to demonstrate the specificity of the migration inhibition by anti-CCL21 (FIG. 12H).

### EXAMPLE 13: Production of IL-12 p70 by Viruses in In Vivo Mouse Studies

To systemically evaluate virus and transgene protein production and activities we set up in vivo study in which SCID mice were subcutaneously implanted in one flank with A549 cells (2 million cells mixed with Matrigel, 50:50 ratio). Once tumours reached a volume of ˜200 m m³, mice were randomised into different groups (7mice per group) and treated intravenously on each of day 0, 2 and 5, with 5×10⁹ virus particles of EnAd, NG-786A, NG-791A or NG-796A viruses (or PBS in the ‘no virus’ control). On day 7, a cocktail of labelled cells (effector memory and naïve T cells isolated from PBMCs from healthy donors—using Miltenyi specific kits) responsive to CXCL9 and CCL21 chemokine gradients was injected intravenously. After 48 hours tumour xenografts were analysed for T cell infiltration, transgene RNA expression and chemokine protein expression by ELISA and plasma samples were analysed for IL-12 transgene protein production by ELISA.

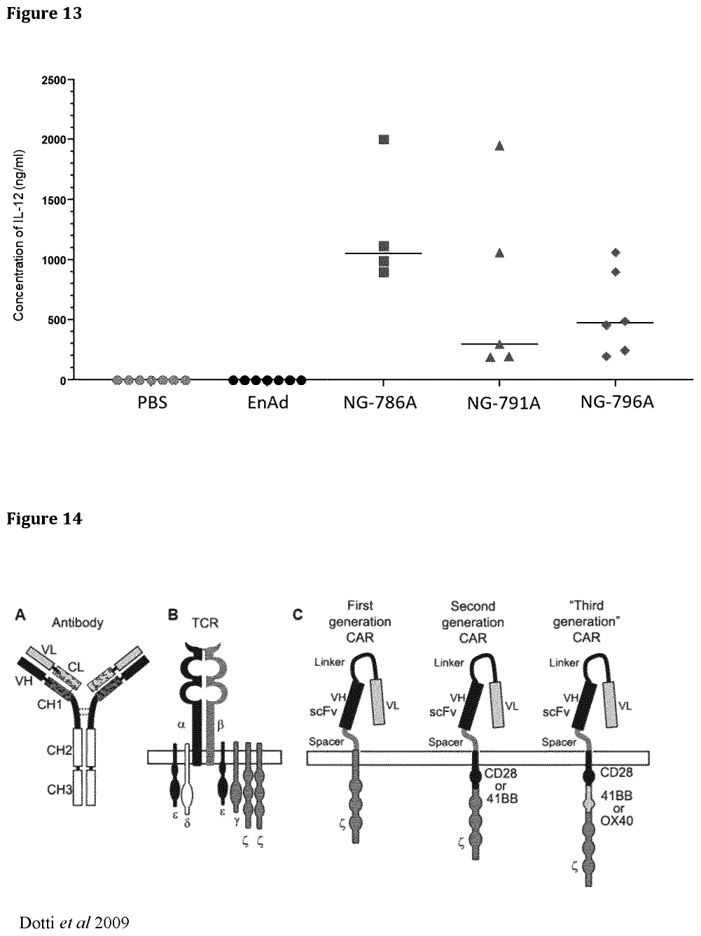

The data in FIG. 13 show that IL-12p70 was found in the blood of all mice treated with IL-12 transgene-encoding viruses, indicating that the viruses had infected tumour cells and expressed the encoded transgenes.

### EXAMPLE 14: In Vitro Migration of Dendritic Cells in Response to CCL21

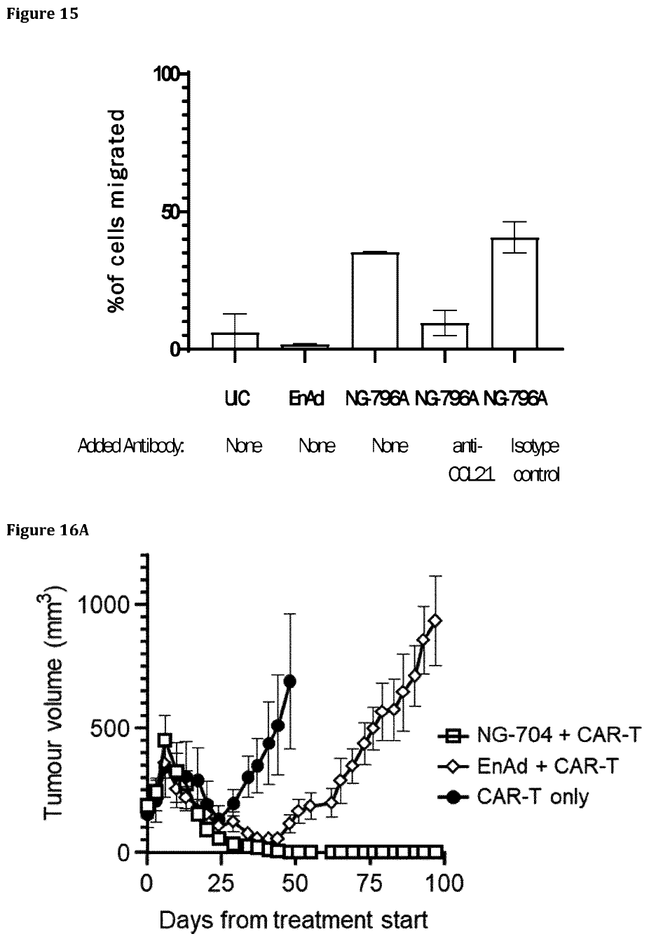

To demonstrate functionality of the CCL21 transgene produced by NG-796A virus, monocyte-derived dendritic cells prepared from PBMCs (by culturing them with GM-CSF and IL-4 as described in Example 3) were stimulated with LPS for 24 before using in a transwell migration assay as described for NG-795A in Example 12. The data in FIG. 15 show that dendritic cell migration was selectively increased by the CCL21-containing supernatant from NG-796A, compared to supernatants from uninfected (UIC) or EnAd infected cells and this was inhibited by the presence of anti-CCL21 blocking antibody (but not by its isotype control).

### EXAMPLE 15: NG-704 and NG-796A Synergy with CAR-T Cells in an In Vivo Mouse Tumour Xenograft Model

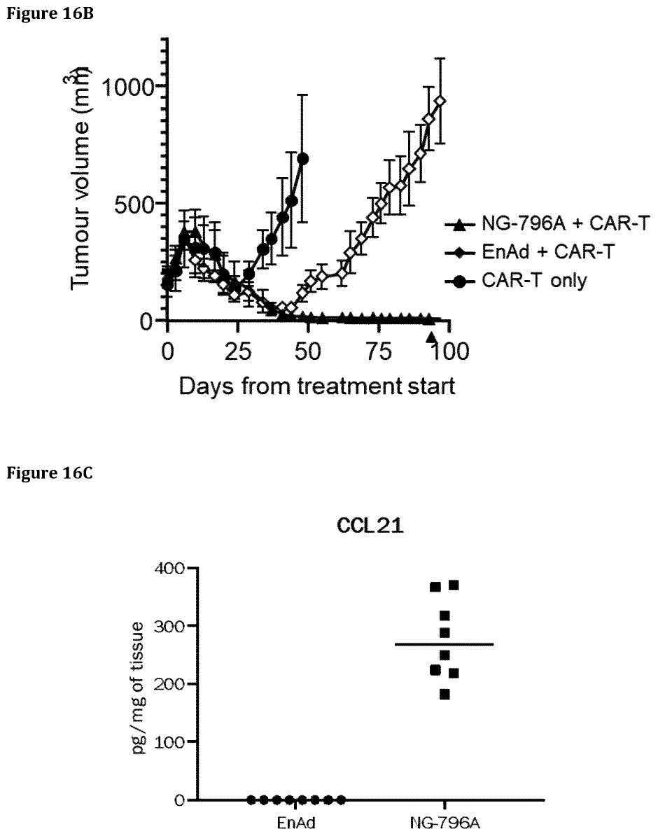

To evaluate virus and transgene activities in a human tumor xenograft system, we set up an in vivo study in which NSG mice were subcutaneously implanted in one flank with 5×10⁶ A549 cells (HER-2 positive). Once tumours reached a volume of ˜200 mm³, mice were randomised into different groups (5 mice per group) and treated intravenously on each of day 0, 3 with 5×10⁹ virus particles of EnAd, NG-704 or NG-796A viruses (or PBS in the ‘CAR-T only’ control). On day 6, 1×10⁷ HER-2 specific CAR-T cells (ProMab) were injected intravenously. Tumours were measured twice per week and tumour growth was followed until the end of the study (˜90 days). The data in FIGS. 16A&B show that CAR-T cells synergised with both NG-704 and NG-796A, respectively, leading to long-term clearance of the tumours. EnAd-treatment only delayed tumour growth compared to CAR-T cells only, indicating that the transgenes encoded by NG-704 and NG-796A are important for anti-tumour activity in the presence of tumour antigen (HER-2)-specific CAR-T cells

### EXAMPLE 16: NG-796A Mediated Production of CCL21 in Human Tumour Xenografts

SCID mice were subcutaneously implanted in one flank with A549 cells (2 million cells mixed with Matrigel, 50:50 ratio). Once tumours reached a volume of ˜200 mm³, mice were randomised into two different groups (8 mice per group) and treated intravenously on each of day 0, 1 and 3, with 5×10⁹ virus particles of EnAd or NG-796A viruses. On day 15, plasma and tumours were collected, tumours lysed and samples analysed for CCL21. by ELISA. The data in FIG. 16C show that CCL21 was found in all of the tumours from mice treated with NG-796A, indicating that the viruses had infected tumour cells and expressed the encoded transgenes. No CCL21 was detected in any plasma samples, indicating that a CCL21 chemokine gradient had been established in the tumours of NG-796A treated mice).

### EXAMPLE 17: Production of IL15-Ra Sushi Domain in Tumour Cell Cultures Infected with NG-796A Virus

A recombinant IL-15Ra sushi domain protein, including the C-terminal P2A peptide and an added N-terminal His tag (SEQ ID NO: 244), was produced and purified by standard E. coli protein expression and purification techniques (Native Antigen Company, Oxford, UK). This recombinant protein was used as a standard in ELISAs for detecting IL-15Ra sushi domain production in transfection and virus infection experiments.

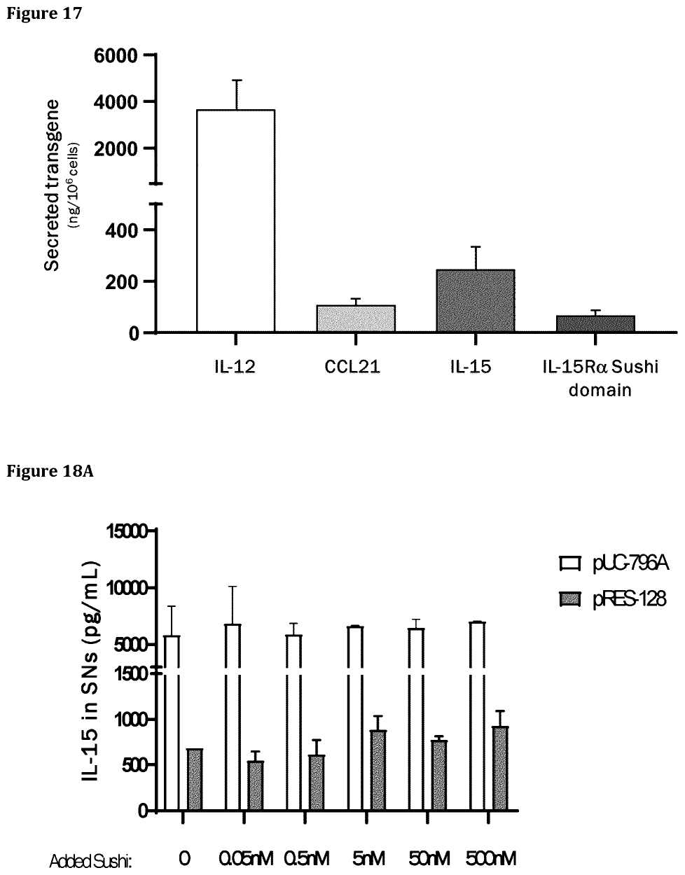

Secretion of IL-15Ra sushi domain (alongside the other encoded transgenes) in supernatants of A549 cells infected with NG-796A virus (or EnAd as a control) was characterised by ELISA (as described in Example 12) using a standard curve of recombinant IL-15Ra sushi domain protein. Results shown in FIG. 17 show that the IL-15Ra sushi domain protein is produced and secreted at similar levels to those of IL-15 cytokine.

### EXAMPLE 18: IL15-Ra Sushi Domain Enhances IL-15 Secretion

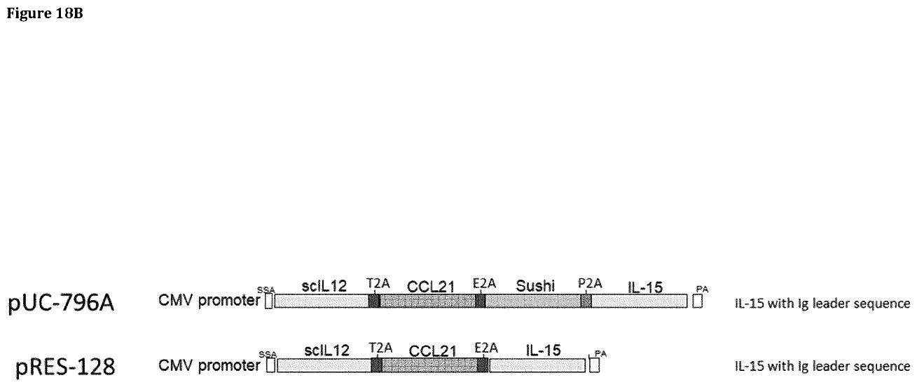

To assess the role of IL-15Ra sushi domain and support its role in the NG-796A virus sequence, we performed transfection experiments using pUC vectors bearing transgene cassettes under control of a CMV promoter, following a similar approach to that described in Example 9. A549 cells were transfected with either pUC-796A (having the transgene cassette sequence of NG-796A; SEQ ID NO: 245) or pRES-128 (a version which lacks the IL-15Ra sushi domain sequence; SEQ ID NO: 246) (see FIG. 18B). Secreted IL-15 cytokine in supernatants was quantified by ELISA. Results shown in FIG. 18A show that encoding the IL-15Ra sushi domain for production by the virus infected cells markedly enhances IL-15 secretion. Addition of recombinant IL-15Ra sushi domain to the cultures, enabling it to interact with any IL-15 released by the cells, led to a small increase in detectable IL-15 indicating that in addition to promoting IL-15 secretion from the cells it may also increase IL-15 stability.

1-29. (canceled)

30. A group B adenovirus comprising a sequence of formula (I):
5′ITR-B₁-B_{A}-B₂-B_{X}-B_{B}-B_{Y}-B₃-3′ITR (I)
wherein:
B₁ is a bond or comprises: E1A, E1 B or E1A-E1 B;
B_{A} comprises-E2B-L1-L2-L3-E2A-L4;
B₂ is a bond or comprises: E3;
B_{X} is a bond or a DNA sequence comprising: a restriction site, one or more transgenes or both;
B_{B} comprises L5;
B_{Y} comprises a sequence -G1-G2ₙ-G3ₘ-G4ₚ-G5_{q},
wherein: G1 is a first transgene; G2 is a second transgene; G3 is a third transgene;
G4 is a fourth transgene; G5 is a fifth transgene,
B₃ is a bond or comprises: E4;
n is 0 or 1; m is 0 or 1; p is 0 or 1; q is 1;
B₃ is a bond or comprises: E4;
wherein:
IL-15 is encoded as a transgene in position G5,
characterised in that B_{Y} also encodes a polypeptide comprising the sushi domain of IL-15R alpha, and does not comprise a transmembrane or GPI anchor such that it is not membrane anchored when expressed.

31. A group B adenovirus according to claim 30, wherein the polypeptide encoding the sushi domain does not comprise a transmembrane domain or GPI anchor.

32. A group B adenovirus according to claim 30, wherein p is 1 and the polypeptide comprising the IL-15R alpha sushi domain is encoded in G4.

33. A group B adenovirus according to claim 30, wherein the leader sequence as employed for IL-15 is the non-native sequence selected independently from Ig, CD33 or IL-2 leader sequence.

34. A group B adenovirus according to claim 30, wherein the virus also encodes IL-12.

35. A group B adenovirus according to claim 34, wherein IL-12 has a sequence shown in SEQ ID NO: 115.

36. A group B adenovirus according to claim 34, wherein IL-12 is encoded in G1 or G2.

37. A group B adenovirus according to claim 30, where at least one further cytokine is encoded in B_{Y}.

38. A group B adenovirus according to claim 30, wherein B_{Y} encodes at least one chemokine.

39. A group B adenovirus according to claim 38, wherein the chemokine is selected from CXCL9, CCL19 and CCL21.

40. A group B adenovirus according to claim 38, wherein the chemokine is encoded in G1, G2 and/or G3.

41. A group B adenovirus according to claim 30, wherein m is 0.

42. A group B adenovirus according to claim 30, wherein the one or more transgenes (such as all transgenes) in B_{Y} are under the control of the major later promoter.

43. A group B adenovirus according to claim 30 wherein the virus comprises a DNA sequence encoding a protein sequence selected from SEQ ID NO: 175 to 189.

44. A composition comprising a group B adenovirus according to claim 30 and a pharmaceutically acceptable excipient, diluent or carrier.

45. A method of treating cancer, comprising administering a therapeutically effective amount of a group B adenovirus according to claim 30.

46. A combination therapy comprising a virus according to claim 30 and a cellular therapy.
